SEQUENCE LISTING (41 sequences)

SEQ ID NO: 1 - DNA, 1853 bp, Homo sapiens
gcagtgtcactaggccggctgggggccctgggtacgctgtagaccagaccgcgacaggccagaacacgggcggcggcttcgggccgggagacccgcgcagccctcggggcatctcagtgcctcactccccaccccctcccccgggtcgggggaggcggcgcgtccggcggagggttgaggggagcggggcaggcctggagcgccatgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtgacaggtccctgatccgccccagcctgcaggccagaagcagtgttacacacttcctggaggagctaaggaaatcctcagactcctgggtttttgttgttgctgttgttgttttttaaaaggtgtgttggcatataatttatggtaatttattttgtctgccacttgaacagtttgggggggtgaggtttcatttaaaatttgttcagagatttgtttcccatagttggattgtcaaaaccctatttccaagttcaagttaactagctttgaatgtgtcccaaaacagcttcctccatttcctgaaagtttattgatcaaagaaatgttgtcctgggtgtgttttttcaatcttctaaaaaataaaatctggaatcctgaaaaaaaaaaaaaaaaaaaaaaaaaaaa

SEQ ID NO: 2 - DNA, 1245 bp, Homo sapiens
atgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtga

SEQ ID NO: 3 - DNA, 1245 bp, Homo sapiens; identical to SEQ ID NO: 2

SEQ ID NO: 4 - DNA, 488 bp, Homo sapiens
atgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgca

SEQ ID NO: 5 - protein, 414 aa, Homo sapiens [sequence omitted]

SEQ ID NO: 6 - protein, 162 aa, Homo sapiens
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAERLRVQHMQDHPNYKYRPRRRKQVKRLKRVEGGFLHGLAEP

SEQ ID NO: 7 - protein, 414 aa, Homo sapiens [sequence omitted]

SEQ ID NO: 8 - DNA, 1894 bp, Homo sapiens
gtctgacttcctcccagcacattcctgcactctgccgtgtccacactgccccacagacccagtcctccaagcctgctgccagctccctgcaagcccctcaggttgggccttgccacggtgccagcaggcagccctgggctgggggtaggggactccctacaggcacgcagccctgagacctcagagggccaccccttgagggtggccaggcccccagtggccaacctgagtgctgcctctgccaccagccctgctggcccctggttccgctggccccccagatgcctggctgagacacgccagtggcctcagctgcccacacctcttcccggcccctgaagttggcactgcagcagacagctccctgggcaccaggcagctaacagacacagccgccagcccaaacagcagcggcatgggcagcgccagcccgggtctgagcagcgtatcccccagccacctcctgctgccccccgacacggtgtcgcggacaggcttggagaaggcggcagcgggggcagtgggtctcgagagacgggactggagtcccagtccacccgccacgcccgagcagggcctgtccgccttctacctctcctactttgacatgctgtaccctgaggacagcagctgggcagccaaggcccctggggccagcagtcgggaggagccacctgaggagcctgagcagtgcccggtcattgacagccaagccccagcgggcagcctggacttggtgcccggcgggctgaccttggaggagcactcgctggagcaggtgcagtccatggtggtgggcgaagtgctcaaggacatcgagacggcctgcaagctgctcaacatcaccgcagatcccatggactggagccccagcaatgtgcagaagtggctcctgtggacagagcaccaataccggctgccccccatgggcaaggccttccaggagctggcgggcaaggagctgtgcgccatgtcggaggagcagttccgccagcgctcgcccctgggtggggatgtgctgcacgcccacctggacatctggaagtcagcggcctggatgaaagagcggacttcacctggggcgattcactactgtgcctcgaccagtgaggagagctggaccgacagcgaggtggactcatcatgctccgggcagcccatccacctgtggcagttcctcaaggagttgctactcaagccccacagctatggccgcttcattaggtggctcaacaaggagaagggcatcttcaaaattgaggactcagcccaggtggcccggctgtggggcatccgcaagaaccgtcccgccatgaactacgacaagctgagccgctccatccgccagtattacaagaagggcatcatccggaagccagacatctcccagcgcctcgtctaccagttcgtgcaccccatctgagtgcctggcccagggcctgaaacccgccctcaggggcctctctcctgcctgccctgcctcagccaggccctgagatgggggaaaacgggcagtctgctctgctgctctgaccttccagagcccaaggtcagggaggggcaaccaactgccccagggggatatgggtcctctggggccttcgggaccatggggcaggggtgcttcctcctcaggcccagctgctcccctggaggacagagggagacagggctgctccccaacacctgcctctgaccccagcatttccagagcagagcctacagaagggcagtgactcgacaaaggccacaggcagtccaggcctctctctgctccatccccctgcctcccattctgcaccacacctggcatggtgcagggagacatctgcacccctgagttgggcagccaggagtgcccccgggaatggataataaagatactagagaactg

SEQ ID NO: 9 - DNA, 1008 bp, Homo sapiens
atgggcagcgccagcccgggtctgagcagcgtatcccccagccacctcctgctgccccccgacacggtgtcgcggacaggcttggagaaggcggcagcgggggcagtgggtctcgagagacgggactggagtcccagtccacccgccacgcccgagcagggcctgtccgccttctacctctcctactttgacatgctgtaccctgaggacagcagctgggcagccaaggcccctggggccagcagtcgggaggagccacctgaggagcctgagcagtgcccggtcattgacagccaagccccagcgggcagcctggacttggtgcccggcgggctgaccttggaggagcactcgctggagcaggtgcagtccatggtggtgggcgaagtgctcaaggacatcgagacggcctgcaagctgctcaacatcaccgcagatcccatggactggagccccagcaatgtgcagaagtggctcctgtggacagagcaccaataccggctgccccccatgggcaaggccttccaggagctggcgggcaaggagctgtgcgccatgtcggaggagcagttccgccagcgctcgcccctgggtggggatgtgctgcacgcccacctggacatctggaagtcagcggcctggatgaaagagcggacttcacctggggcgattcactactgtgcctcgaccagtgaggagagctggaccgacagcgaggtggactcatcatgctccgggcagcccatccacctgtggcagttcctcaaggagttgctactcaagccccacagctatggccgcttcattaggtggctcaacaaggagaagggcatcttcaaaattgaggactcagcccaggtggcccggctgtggggcatccgcaagaaccgtcccgccatgaactacgacaagctgagccgctccatccgccagtattacaagaagggcatcatccggaagccagacatctcccagcgcctcgtctaccagttcgtgcaccccatctga

SEQ ID NO: 10 - protein, 335 aa, Homo sapiens
MGSASPGLSSVSPSHLLLPPDTVSRTGLEKAAAGAVGLERRDWSPSPPATPEQGLSAFYLSYFDMLYPEDSSWAAKAPGASSREEPPEEPEQCPVIDSQAPAGSLDLVPGGLTLEEHSLEQVQSMVVGEVLKDIETACKLLNITADPMDWSPSNVQKWLLWTEHQYRLPPMGKAFQELAGKELCAMSEEQFRQRSPLGGDVLHAHLDIWKSAAWMKERTSPGAIHYCASTSEESWTDSEVDSSCSGQPIHLWQFLKELLLKPHSYGRFIRWLNKEKGIFKIEDSAQVARLWGIRKNRPAMNYDKLSRSIRQYYKKGIIRKPDISQRLVYQFVHPI

SEQ ID NO: 11 - DNA, 2258 bp, Mus musculus
gcaccttccatgcttggtactgcccagatcttggcctcctcaaagagccttttgattccctaattcatcccaccagagtgtctatcccctgtgacatctgccaggcacagactcaaaccctaagccatccatccactggggaactagtcctgtccatctggtgttggctggctggctgcacagtttgttttagggcctgaacacctgtgtgtatgtgtgtgtgtgtgtgtgtgtgtatgtgtgtgtgtgcgtgcgtgtgctctgtatgtgccatctgtgacccctctgtatgtgccatctgtgtacccctcacattcatggagtacacttataggcacattacagtggggacatcaagatgcaatatttatggattgctgcatgattcttaaaagtgaataaaactgtttacaggggcaggggcgccattaattacaacgtgaattcgtttgtgaacgtattcgtttcagtcagtggctgaggctctttttagtatttctgcagacctgggacaaggccagctccattcctgactgccctacctgcttctgcatctgccttaaaacccgatcacttccaatcctgcagctcgtcaattattaaagaaccccaaagctgatggcaatagggaaagctagggcccagaatcccttatgcttagtagttccatcacttaaccagttcctggctaaatcagtagaacaaatgacctctttgagaatcttgcaggcccagggcttttctgttttttgccggctggctctgcttcctagactgtctgaaagggcagcacctccgcagatgcccctggaatgccagcttccgggaacttgagcagggtgagtgggcgagctgcttaccgggagacgctcccttaccctacccgcctcggtgtgtctgcgagctgtgtggccgtttctgcggattataagcgacaggtggcatcagagagtcaaacttgcctgtgggcaccagagaagacactcggggaccagggaaaggccccagaaaccagacaaaggccactggcagcccggtgagcgaaagacttcaagggcacggttccccgagacttggtctgcagaatccggggtctgggtccctcagcggtcaaacccgacgcgttgggggaggggttcccggggcctcgagcgggagccgagcacccgccgtgagacccagcgtcggggccctcgggtcgggaatgcacagagtcggatctcggtggcgggtagggggctgggtggcgtcgggagagggcggcagaaccagagagttccagagagttgccgccagaggagctgcaagggcccgcggtctccttggagcagtactggccaatgacagcagcggaacggttttatgggggcggggcgtcgcatggcatggcagcagaagaagccttccgagcgtctacttaatgccggtccccggccgtcgctcagagaagcccgtcgggcgaggggggggggcgcgaagaggggcgccagcacagccctcgagctggggacagagaaccgcgggaggtgggaggaacttcttccagaaccttcctctggctggctgcgctctgagccaggtaagggactgggagggctgtggaccacgggatgggagggcgtcgggacctgctggcttaccagtccatcggtcgcccacagaagtagtggggccacgaaaatgaaatctgaggagttcgggagatgcttcctgctcctccataactcggtcctgtgaccttcctttcccgagacccttcatcctaacacctcttttcaaggatcagatgacctactttgccccagccgcgtcgtactgacccccctggactattggatgtgatcttagtttcccactctttggggaaaagagttagtttgggccttcctaccctcttccctcattgtggccaacaaacccttcaagggcagatggagagaggtggagacgcaggatcagaccagggtcctgtgtttccctgacctcgccacaggctgcggggatgaatgtgaggggctccccccctaaggcgcctctttccctctcggggtaggggaagagtgttcagttccgtggtgctcccgcaggcctcacattcgtgttcggaggaggcgaccccctgggtcgcagaatggaagtgagtgttctggagactgtgtaactggcgtctgggttaccaacgggccagagagcgcggcggccactccggctcagcctgtggatgttgaccgccccttcggagagtccccgcagacatg

SEQ ID NO: 12 - DNA, 24 bp, Artificial Sequence
  Synthetic nucleic acid sequence
ggtaccaaagacttcaagggcacg

SEQ ID NO: 13 - DNA, 26 bp, Artificial Sequence
  Synthetic nucleic acid sequence
agatctgccagttacacagtctccag

SEQ ID NO: 14 - DNA, 22 bp, Artificial Sequence
  Synthetic nucleic acid sequence
cttctgacagcaggcggctaac

SEQ ID NO: 15 - DNA, 27 bp, Artificial Sequence
  Synthetic nucleic acid sequence
gactggatgcacaaattggtagacaag

SEQ ID NO: 16 - DNA, 28 bp, Artificial Sequence
  Synthetic nucleic acid sequence
ttgacgcgtgttatcttagagtccgccg

SEQ ID NO: 17 - DNA, 27 bp, Artificial Sequence
  Synthetic nucleic acid sequence
aaactcgagatggctctccagaccgac

SEQ ID NO: 18 - DNA, 23 bp, Artificial Sequence
  Synthetic nucleic acid sequence
tgtccgccttctacctctcctac

SEQ ID NO: 19 - DNA, 21 bp, Artificial Sequence
  Synthetic nucleic acid sequence
cgatgtccttgagcacttcgc

SEQ ID NO: 20 - DNA, 24 bp, Artificial Sequence
  Synthetic nucleic acid sequence
gttgcctgctactgttcccagatg

SEQ ID NO: 21 - DNA, 24 bp, Artificial Sequence
  Synthetic nucleic acid sequence
aaagccacttctgcacgttaccag

SEQ ID NO: 22 - DNA, 21 bp, Artificial Sequence
  Synthetic nucleic acid sequence
cttcaccaccatggagaaggc

SEQ ID NO: 23 - DNA, 21 bp, Artificial Sequence
  Synthetic nucleic acid sequence
ggcatggactgtggtcatgag

SEQ ID NO: 24 - DNA, 38 bp, Artificial Sequence
  Synthetic nucleic acid sequence
cccggggtcgaccgcagcatgggcagtgccagcccagg

SEQ ID NO: 25 - DNA, 37 bp, Artificial Sequence
  Synthetic nucleic acid sequence
cccgggtctagatcagactggatgcacaaattggtag

SEQ ID NO: 26 - DNA, 19 bp, Artificial Sequence
  Synthetic nucleic acid sequence
tgaacatcacagcagaccc

SEQ ID NO: 27 - DNA, 22 bp, Artificial Sequence
  Synthetic nucleic acid sequence
tcttcccattctaaacaacacc

SEQ ID NO: 28 - DNA, 32 bp, Artificial Sequence
  Synthetic nucleic acid sequence
gggcacggatccatgggcagtgccagcccagg

SEQ ID NO: 29 - DNA, 37 bp, Artificial Sequence
  Synthetic nucleic acid sequence
cccggggtcgactcagactggatgcacaaattggtag

SEQ ID NO: 30 - DNA, 20 bp, Artificial Sequence
  Synthetic nucleic acid sequence
ttccaggagctgggcggtaa

SEQ ID NO: 31 - DNA, 24 bp, Artificial Sequence
  Synthetic nucleic acid sequence
ggtccatggtgatacaagggacat

SEQ ID NO: 32 - DNA, 23 bp, Artificial Sequence
  Synthetic nucleic acid sequence
tgagcaacatcacacaagaccag

SEQ ID NO: 33 - DNA, 23 bp, Artificial Sequence
  Synthetic nucleic acid sequence
gagaaaggaaaatgatccacagc

SEQ ID NO: 34 - DNA, 20 bp, Artificial Sequence
  Synthetic nucleic acid sequence
aacccccagctagttgtcat

SEQ ID NO: 35 - DNA, 20 bp, Artificial Sequence
  Synthetic nucleic acid sequence
gctctttaggctttccagga

SEQ ID NO: 36 - DNA, 23 bp, Artificial Sequence
  Synthetic nucleic acid sequence
cctctggtcttctggagtaccat

SEQ ID NO: 37 - DNA, 23 bp, Artificial Sequence
  Synthetic nucleic acid sequence
ggcataacgcactaggtttgccg

SEQ ID NO: 38 - DNA, 24 bp, Artificial Sequence
  Synthetic nucleic acid sequence
cctgttcgctctgggtattgtgtt

SEQ ID NO: 39 - DNA, 24 bp, Artificial Sequence
  Synthetic nucleic acid sequence
cgtggtccgctgatttcttctcta

SEQ ID NO: 40 - DNA, 20 bp, Artificial Sequence
  Synthetic nucleic acid sequence
gaccatgaagctgctgccgt

SEQ ID NO: 41 - DNA, 22 bp, Artificial Sequence
  Synthetic nucleic acid sequence
cgcccaacttcactttctcttc

(19) United States
(12) Patent Application Publication
(10) Pub. No.: US 2011/0177996 A1
(43) Pub. Date: Jul. 21, 2011

(54) REGULATORY PROTEINS IN LUNG REPAIR AND TREATMENT OF LUNG DISEASE

(75) Inventors: Jeffrey A. Whitsett, Cincinnati, OH (US); James Wells, Cincinnati, OH (US)

(73) Assignee: CHILDREN'S HOSPITAL MEDICAL CENTER, Cincinnati, OH (US)

(21) Appl. No.: 11/915,229

(22) PCT Filed: May 23, 2006
(86) PCT No.: PCT/US2006/020512

§ 371 Date: Sep. 25, 2009

Related U.S. Application Data
(60) Provisional application No. 60/684,237, filed on May 23, 2005.

(51) Int. Cl. A61K 38/02; C07K 2/00; C07H 21/00; A61K 31/7088; C12N 5/071; C12Q 1/02; A61P 11/00

(52) U.S. Cl. 514/1.1; 530/350; 536/23.5; 514/44 R; 435/377; 435/29

(57) ABSTRACT

The DNA-binding protein Sox17 has been found to play an important role in repair of pulmonary tissue after damage, disease, or injury. Nucleic acids encoding proteins involved in pulmonary repair, such as Sox17 and Spdef, can be used as a therapeutic composition for treating pulmonary disease. Methods of treatment of pulmonary injuries or pulmonary diseases are also disclosed, as are methods of identifying compounds effective in treating pulmonary injuries and diseases.

### FIELD OF THE INVENTION

The invention relates to the treatment of pulmonary injury. More specifically, aspects of the invention relate to the finding that the DNA-binding protein Sox17 is capable of inducing the activation of the pulmonary repair system.

### BACKGROUND OF THE INVENTION

During an individual's lifetime, the lung is repeatedly subjected to injury by various pathogens and toxicants throughout the lifetime of the individual. The cell surface of the respiratory tract is directly exposed to inhaled gases, particles, and pathogens. A complex epithelium derived from foregut endoderm lines the airways and mediates gas exchange, mucociliary clearance, host defense, and surfactant homeostasis to maintain lung sterility and stability. The mature lung responds to various injuries by undergoing proliferation to repair epithelial cell surfaces and maintain lung function.

Pulmonary repair after an injury involves complex molecular pathways. Several of the proteins involved in this process have now been identified, and are described herein. Among these proteins is the Sox protein Sox17. Sox proteins are a subfamily of the DNA-binding protein superfamily called “High Mobility Group” (HMG) proteins. The Sox protein subfamily exhibits similarity to the HMG protein Sry. The HMG domain of the Sox family is thus termed the “Sry box” (Wegner et al., Nucleic Acids Research, 27:1409-1420 (1999), which is incorporated by reference herein in its entirety) family of DNA-binding proteins. Several Sox proteins have been identified in various organisms, where they are involved in diverse developmental processes.

Another protein that is involved with pulmonary pathways is Spdef. Spdef is expressed in pulmonary epithelial cells, and also regulates gene expression in respiratory epithelia cells.

### SUMMARY OF THE INVENTION

The pulmonary system provides homeostasis and repair of the lung in response to attack by pathogens, toxins, pollutants, and other types of injuries. The research described herein demonstrates that following extensive injury to the conducting airway epithelium in the mouse, endodermally-derived, ciliated cells underwent rapid transdifferentiation into squamous progenitor cells that spread to repair the injured airways. The squamous cells underwent a columnar cell transition, as the diverse differentiated cell types of the airway epithelium were restored. Enhanced expression of Sox17 coincided with that of β-catenin and Stat-3, that together, preceded widespread expression of transcription factors critical for lung epithelial cell differentiation, including TTF-1, Foxa2, and Foxj1. While induction of Sox17 and squamous metaplasia occurred in the absence of β-catenin, restoration of diverse epithelial cell types required β-catenin. Additionally, the expression of Sox17 in respiratory epithelial cells of transgenic mice induced β-catenin and Stat-3, and resulted in the formation of clusters of cuboidal-columnar epithelial cells of diverse airway epithelial cell types. Further, β-catenin and Sox17 are shown to participate in a transcriptional program influencing progenitor cell behavior during repair of the airway epithelium. Accordingly, in some embodiments of the invention, alteration of pulmonary levels of Sox17, Spdef, β-catenin, Stat-3, and other proteins described herein can be used to treat or repair pulmonary tissue.

Thus, a better understanding of the proteins and molecular pathways involved in pulmonary cell proliferation can be useful for devising pharmaceutical compounds for the treatment of lung damage.

In an embodiment of the present invention, a pharmaceutical composition effective in treating lung injury in a mammal is provided, having a nucleic acid encoding Sox17 protein, or a fragment thereof, in admixture with a pharmaceutically acceptable excipient.

In another embodiment of the present invention, a pharmaceutical composition effective in treating lung injury in a mammal is provided, having a nucleic acid having at least 90%, 95%, 97%, 98%, or 99% homology to a nucleic acid encoding human Sox17 protein or a fragment thereof, in admixture with a pharmaceutically acceptable excipient. The nucleic acid fragment can be, for example, at least 50, 100, 150, 200, 250, 500, 800, 1000, or 1240 nucleotides in length.

In an embodiment of the present invention, a method for the treatment of lung injury is provided, by introducing a composition having a nucleic acid encoding mammalian Sox17 protein or fragment thereof, to a human in an amount effective to reduce the symptoms of the lung injury. The expression of β-catenin can be activated. The expression of Stat-3 can be activated. The composition can be administered intratracheally. The composition can be administered by aerosolization. The composition can be administered using a nebulizer. The lung injury can be a chemically-induced lung injury. The lung injury can be caused by a pulmonary disease. The lung injury can be caused by at least one condition selected from the group consisting of: pulmonary fibrosis, sarcoidosis, asbestosis, aspergilloma, aspergillosis, pneumonia, pulmonary tuberculosis, rheumatoid lung disease, bronchiectasis, bronchitis, bronchopulmonary dysplasia, interstitial lung disease, occupational lung disease, emphysema, cystic fibrosis, acute respiratory distress syndrome (ARDS), asthma, chronic bronchitis, and COPD (chronic obstructive pulmonary disease). The lung injury can be caused by a viral, bacterial, or fungal disease. Stat-3 protein, β-catenin or fragment thereof, or a nucleic acid encoding a Stat-3 protein or fragment thereof, can also be introduced.

In another embodiment of the present invention, a method for the treatment of lung injury is provided, by introducing a nucleic acid having at least 90%, 95%, 97%, 98%, or 99% homology to SEQ ID NO: 5 or a fragment thereof, in admixture with a pharmaceutically acceptable excipient.

In an embodiment of the present invention, a method of inducing respiratory epithelial cell differentiation is provided, by administering a nucleic acid encoding a Sox17 polypeptide or fragment therof.

In an embodiment of the present invention, a method of inducing pulmonary progenitor cells to enhance pulmonary repair is provided, by administering a nucleic acid encoding a Sox17 polypeptide or fragment thereof.

In an embodiment of the present invention, a method of treating a pulmonary injury is provided, by administering an agent that upregulates Sox17 expression to an individual. The agent can be, for example, an Spdef protein, a fragment of an Spdef protein, or a nucleic acid encoding Spdef.

In an embodiment of the present invention, a method of identifying a compound for the treatment of pulmonary injury is provided, by obtaining a mammalian cell, testing the cell by adding at least one test compound, and determining whether Sox17 expression is increased, where an increase in Sox17 expression indicates that the test compound is potentially useful for the treatment of pulmonary injury.

In an embodiment of the present invention, a pharmaceutical composition effective in treating lung injury in a mammal is provided, having a nucleic acid encoding Spdef protein, or a fragment thereof, in admixture with a pharmaceutically acceptable excipient.

In an embodiment of the present invention, a pharmaceutical composition effective in treating lung injury in a mammal is provided, having a nucleic acid having at least 90%, 95%, 97%, 98%, or 99% homology to a nucleic acid encoding human Spdef protein or a fragment thereof, in admixture with a pharmaceutically acceptable excipient. The nucleic acid fragment can be, for example, at least 50, 100, 150, 200, 250, 500, 800, 900, or 1000 nucleotides in length. The composition can be administered intratracheally. The composition can be administered by aerosolization. The composition can be administered using a nebulizer. The lung injury can be a chemically-induced lung injury. The lung injury can be caused by a pulmonary disease. The lung injury can be caused by at least one condition selected from the group consisting of: pulmonary fibrosis, sarcoidosis, asbestosis, aspergilloma, aspergillosis, pneumonia, pulmonary tuberculosis, rheumatoid lung disease, bronchiectasis, bronchitis, bronchopulmonary dysplasia, interstitial lung disease, occupational lung disease, emphysema, cystic fibrosis, acute respiratory distress syndrome (ARDS), asthma, chronic bronchitis, and COPD (chronic obstructive pulmonary disease). The lung injury can be caused, for example, by a viral, bacterial, or fungal disease.

In an embodiment of the present invention, a method for the treatment of lung injury is provided, by introducing a composition by a nucleic acid encoding mammalian Spdef protein or fragment thereof, to a human in an amount effective to reduce the symptoms of the lung injury.

In an embodiment of the present invention, a method of identifying a compound for the treatment of pulmonary injury is provided, by obtaining a mammalian cell, testing the cell by adding at least one test compound, and determining whether Spdef expression is increased, whereby an increase in Spdef expression indicates that the test compound is potentially useful for the treatment of pulmonary injury.

### BRIEF DESCRIPTION OF THE DRAWINGS

FIG. 1 is a microscopic image of Lung epithelial origin and ultrastructure of squamous progenitor cells. (a) Hematoxylin-eosin staining of mouse bronchioles 24 hours after naphthalene administration. (b) Green fluorescent protein (GFP) was observed in the squamous progenitor cells lining the conducting airway in hSP-C-rtTA/(tetO)7CMVCre/ZEG mouse 24 hours after naphthalene treatment. (c) Electron micrograph after naphthalene treatment, showing squamous cells with few cilia (black arrowhead). (d) Basal bodies (white arrowhead) and internalized cilia are present with the squamous cells. Scale bar: 2 μm.

FIG. 2 is a microscopic immunofluorescence image showing that ciliated cells can serve as progenitor cells during repair of airway epithelium. Double immunolabeling for clara cell secretory protein (CCSP) (red) and β-tubulin(green) (a-e), and Foxj1 staining (f-j) was performed on lung sections of uninjured control (a, f) and naphthalene treated mice 1-14 days after injection. Sections were counter-stained with 4′,6-diamidino-2-phenylindole (DAPI) (blue, a-e). Figures are representative of n≧5 individual animals.

FIG. 3 is a microscopic image showing dynamic changes in expression of Foxa1, Foxa2, and TTF-1 during repair. Clara cells are indicated by white arrowhead in the inset (d 0).

FIG. 4 is a microscopic image demonstrating Sox17 and β-catenin staining in progenitor cells during repair of airway epithelium. Figures are representative of n≧5 individual animals at each time point.

FIG. 5 is a microscopic image demonstrating the expression of Sox17, β-catenin, Foxa2, Foxj1, and CCSP/β-tubulin during compensatory growth following pneumonectomy. Immunohistochemistry was performed on the right lung 3 days (a, c, e) and 7 days (b, d, f, g, h) after left pneumonectomy. Phosphohistone-3 immunostaining (red in g inset) was detected in β-tubulin(green) positive ciliated cells (arrow, g inset). Adjacent non-ciliated cells (arrowhead) showed no staining or were less intensely labeled with phosphohistone-3 antibody (g inset). Scale bars=25 μm.

FIG. 6 is a microscopic image demonstrating that Sox17 can induce Foxj1 and β-catenin in vivo. Expression of Foxj1, β-catenin, and Sox17 was assessed in lungs from transgenic fetal mice expressing Sox17 (hSP-C-rtTA/(tetO)7Sox17) at E18.0. The figures are representative of 3 separate control and transgenic mice. Scale bars=100 μm.

FIG. 7 is a bar graph demonstrating that Sox17 activated the mouse Foxj1 promoter in vitro. HeLa cells were transfected with increasing amounts of PCIG-Sox17 and PCIG-tSox17. Cotransfection with Sox17 increased the luciferase activity in a dose-dependent manner, while tSox17 was inactive. Results are presented as fold increase in activity compared to the control. Values are mean±S.D., n=3, Data are representative of two separate experiments. P values were obtained by ANOVA.

FIG. 8 is a microscopic image demonstrating that Sox17 induced multiple proximal airway epithelial cell types and caused focal alveolar hyperplasia in vivo. Immunostaining was performed on lung sections of transgenic mice untreated (a-c) or treated (d-o) with dox. Scale bar=25 μm.

FIG. 9 shows phosphohistone-3 staining during repair of the respiratory epithelium. Phosphohistone-3 (pH3) immunostaining was performed on the lung sections of control (day 0) and naphthalene-injected mice (days 1-4) to identify proliferating cells. pH3 staining was not detected in the squamous cells (arrow) lining the injured bronchioles 24 hours after injury (day 1). Cuboidal epithelial cells were positive for pH3 staining 2 days after injury (day 2). Fewer pH3 stained cells were observed thereafter (day 4). Sloughed cells stained non-specifically (arrowhead).

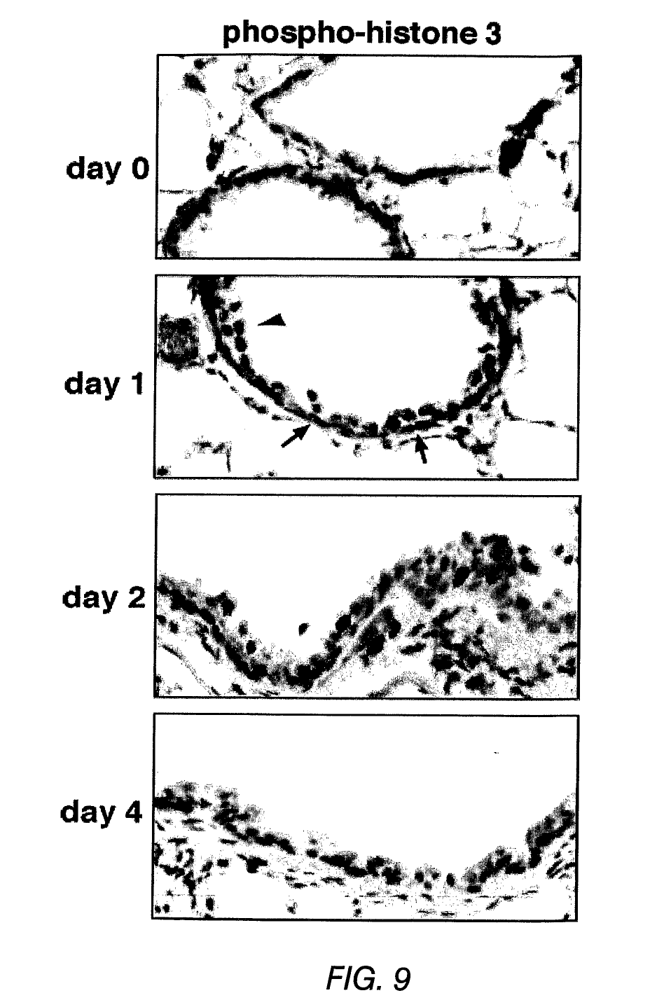

FIG. 10 demonstrates that truncated Sox17 did not alter differentiation of the respiratory epithelium. Immunohistochemistry for Foxj1 and CCSP was performed on lung sections of the adult transgenic mice expressing a truncated form of Sox17 under the control of the rat CCSP promoter. tSox17 staining was observed in a subset of the peripheral respiratory epithelial cells (FIG. 10a). Foxj1 and CCSP staining were not altered in the respiratory epithelial cells expressing the transgene, but were present in the normal in the conducting airway (FIG. 10b, c). Unlike Sox17, tSox17 did not cause focal alveolar hyperplasia.

FIG. 11 shows Spdef mRNA in the respiratory epithelial cells, trachea and tracheal glands in mouse. (A) Spdef and GAPDH mRNA was identified by RT-PCR using RNA extracts from human cells H441, HeLa, HTEpC; MLE-12 cells, mouse lung (m Lu) and trachea (m Tray. Spdef mRNA was detected in H441 and HTEpC, but not in HeLa cells. Spdef mRNA was detected in mouse lung and trachea, but not in MLE-12 cells. PCR without RT (−) showed no product. H441 is a human lung adenocarcinoma cell line; HeLa, a cervical adenocarcinoma cell line; HTEpC, normal human tracheal epithelial cells; MLE-12 cells, an SV40 large T immortalized mouse lung epithelial cell line. In situ hybridization for Spdef mRNA was performed on sections of trachea and tracheal glands (B, D) and lung (C, E) in adult mice. Spdef mRNA was detected in the epithelium lining trachea, bronchi, and tracheal glands (arrows), but not in bronchioles or blood vessels. Inset shows phase microscopy of the hybridized tracheal glands. Scale bars: 200 μm. B, bronchi; Br, bronchioles; V, vessels.

FIG. 12 demonstrates Spdef mRNA in trachea and conducting airways. In situ hybridization for Spdef mRNA was performed on sections of trachea and lungs from fetal (A) and postnatal (B-D) mice. Spdef mRNA is detected in the tracheal epithelium E17.5 (A and inset) and the bronchi at postnatal days 5 (B), 10 (C), and 20 (D), not in peripheral lungs (In) and blood vessels (V). C, Cartilage. Scale bars: A-D, 200 μm; A inset, 50 μm.

FIG. 13 shows Spdef immunohistochemistry staining in mouse trachea and tracheal glands. Immunohistochemistry was performed on sections of adult lungs. Spdef staining was detected in nuclei in epithelial cells lining trachea (A) and tracheal glands. Immunohistochemistry for Spdef (A) of TTF-1 (C), Sox17 (D), Foxj1 (E), and Scgb1a1 (F) is shown in tracheal epithelium. Scale bar=50 μm.

FIG. 14 shows Spdef and TTF-1 activate gene transcription in vitro. Reporter assays were performed using plasmids expressing Spdef and TTF-1 and reporter plasmids in which firefly luciferase gene is controlled by Sftpa (A), Foxj1 (B), Scgb1a1 (C), and Sox17 (D) gene promoters as described in Materials and Methods. Spdef activated Sftpa, Foxj1, Scgb1a1 and Sox17 promoters in presence and absence of TTF-1. Assays were repeated in triplicate in at least three experiments with similar results. Values are mean±SD, n=3. p values were obtained by ANOVA, compared with cells transfected with the reporter and empty expression plasmids.

FIG. 15 demonstrates that Spdef interacts with TTF-1 via the C-terminal domain of TTF-1. (A) Mammalian two-hybrid assay was performed using the luciferase reporter pG5 luc and pACT and pBIND vector system (Promega, Madison, Wis.). Full length and a series of TTF-1 deletion mutants (described in Table 1) were inserted to pBIND vector. Recombinant plasmids were co-transfected with the pG5 luc plasmids and activity compared with that of cells transfected with pACT-Spdef+pBIND-TTF-1. Values are mean±SD, n=3. Assays were repeated three times with similar results. (B) GST pull-down assays were performed with GST-Spdef that was immobilized on glutathione-Sepharose beads. Protein extracts were prepared from HeLa cells transiently transfected with the expression plasmids encoding for 3XFLAG-TTF-1, 3XFLAG-Δ14, and 3XFLAG-Δ3. The extracts were incubated with GST or GST-Spdef. Both GST and GST•Spdef beads were washed several times before boiling, run on 10% SDS-polyacrylamide gels, and analyzed by immunoblot using a monoclonal antibody that recognizes the FLAG sequences.

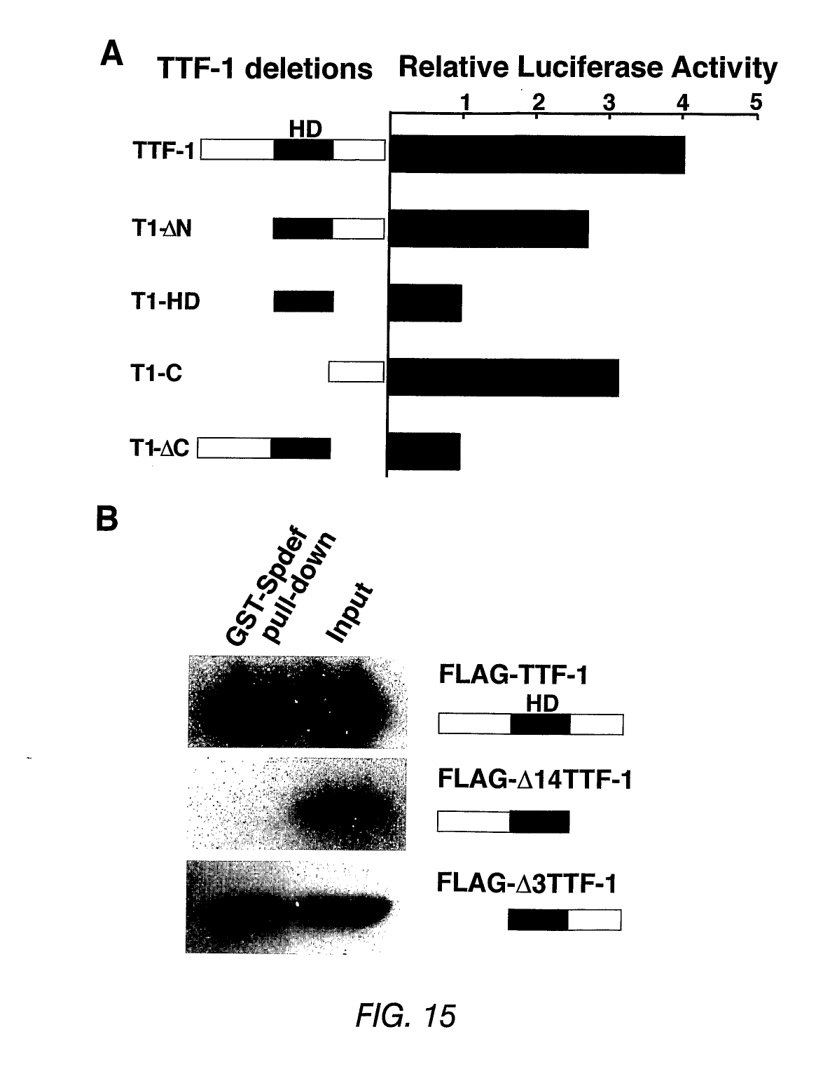

FIG. 16 displays comparison of Spdef and Erm on gene transcription. Reporter assays were performed using plasmids expressing Spdef and Erm, an Ets family transcription factor also expressed in the lung. The plasmids were co-transfected with the firefly luciferase reporter plasmids under control of Sftpa (A), Foxj1 (B), Scgb1a1 (C), and Sox17 (D) gene promoters. While Spdef activated the Sftpa, Foxj1, Scgb1a1, and Sox17 promoters, Erm was less active. Values are mean±SD, n=3; p values obtained by ANOVA. Each assay was repeated in at least three experiments with similar results.

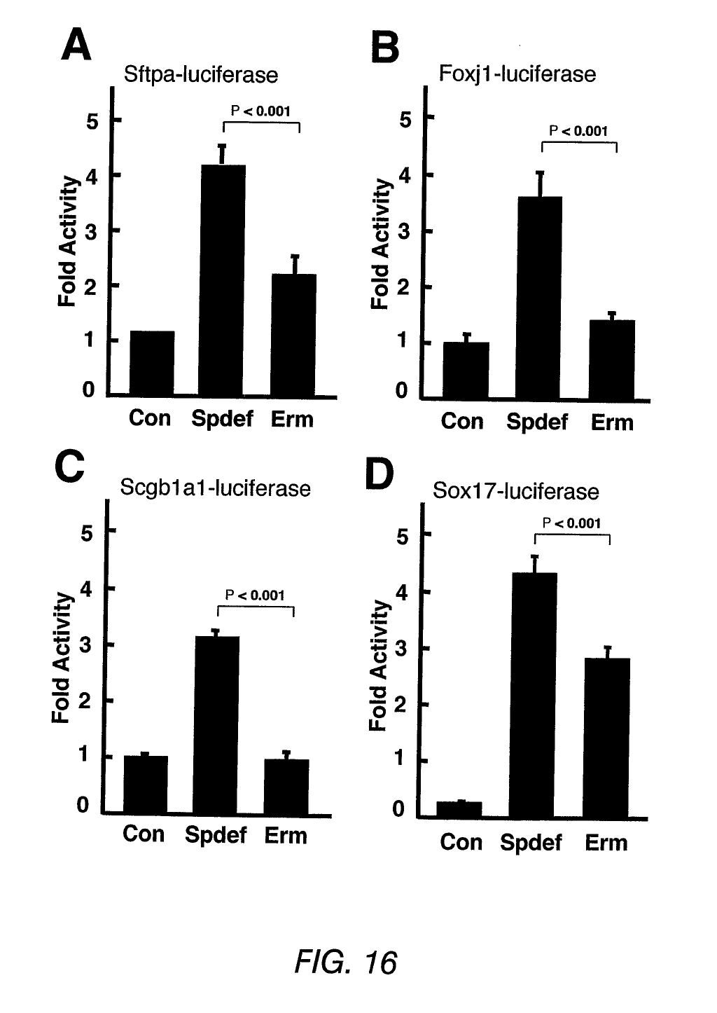

FIG. 17 shows conditional expression of Spdef in vivo. The construct and strategy used to express Spdef in Clara cells in the conducting airway is seen in (A). Transgene specific Spdef mRNA was detected by RT-PCR in whole lung in the presence (+) but not in the absence of doxycycline (DOX) (−) (B). Spdef mRNA was increased in the presence of doxycycline assessed by RT-PCR of whole lung mRNA using primers that selectively detect transgenic Spdef mRNA. GAPDH was detected as an internal control. In situ hybridization (C, D) and immunostaining (G, H) were performed to detect the expression of the Spdef mRNA and protein in conducting airways and lung parenchyma in the absence (C, E, G) and the presence (D, F, H) of doxycycline. Serial sections for C and D were stained with hematoxylin-eosin (E, F). Spdef mRNA and protein were detected at the sites of goblet cell morphology in the conducting airways of CCSP-rtTA/TRE2-Spdef mice treated with doxycycline (DOX) (D, F, H), but were not detected in the bronchiolar epithelium of the transgenic mice without DOX (C, E, G). Scale bars: 200 μm.

FIG. 18 demonstrates that the expression of Spdef caused goblet cell hyperplasia in the conducting airways. CCSP-rtTA/TRE2-Spdef mice were maintained with or without doxycycline (dox) from E0 to PN14. Lung sections were stained with Alcian-blue (A, B) or by immunohistochemistry for Muc5A/C (C, D) and CCSP (E, F). Increased Alcian-blue and Muc5A/C staining was readily detected in the conducting airways of mice in the presence (B, D), but not in the absence (A, C) of doxycycline. Expression of Spdef caused decreased CCSP staining (F and inset), compared to controls without doxycycline (E and inset). Scale bars: A-F, 200 μm.

FIG. 19 shows Foxj1 and loss of Foxa2 staining in lungs of CCSP-rtTA/TRE2-Spdef transgenic mice. Immunohistochemistry for Foxj1 (A, B) and Foxa2 (C, D) was performed on the lung sections of control (A, C) and the transgenic mice expressing Spdef (B, D). The normal staining pattern of Foxj1, a ciliated cell marker, was unaltered by expression of Spdef (A, B). Foxa2 staining was not detected in the goblet cells lining the conducting airways of the transgenic mice (C, D). Scale bars: 50 μm.

FIG. 20 shows that IL-13 induces expression of Spdef. At 5 weeks of age, CCSP-rtTA/tetO-CMV-IL-13 mice were maintained with or without doxycycline (DOX) for 1 week. RT-PCR for Spdef was performed using total RNA from the transgenic mice (A). Spdef mRNA was increased in the transgenic mice treated with DOX, while GAPDH was unchanged. In situ hybridization (B) and Spdef immunostaining (C) were performed on lung sections from the transgenic mice. Spdef mRNA was induced in the conducting airways of the transgenic mice treated with DOX, and was not detected in the mice without DOX (B). Spdef staining was detected in the conducting airways of the transgenic mice treated with DOX (C), consistent with the sites of Spdef mRNA expression. Spdef was not detected in the absence of doxycycline. Scale bars 200 μm.

FIG. 21 shows that IL-13 and dust mite allergen induce Spdef and cause goblet cell hyperplasia. Immunohistochemistry for Spdef was performed on lung sections of control (A) and Stat-6^{-/-} (B) mice that were treated intratracheally with IL-13. Spdef staining was increased at sites of goblet cell hyperplasia; staining was absent in conducting airways of Stat-6^{-/-} mice. Spdef was increased in association with goblet cell hyperplasia caused by intratracheal exposure to house dust mite allergens in wild type mice (C), but not in the conducting airways of exposed IL-13^{-/-} mice (D). Scale bars: 200 μm.

FIG. 22 demonstrates that Spdef mRNA was detected in various tissues in the mouse. In situ hybridization for Spdef mRNA was performed on sections of adult mouse tissues. Spdef mRNA was detected in the epithelium of the dorsal (A) and ventral (B) prostate coagulating gland, seminal vesicle (C), stomach (not shown), small intestine (not shown), colon (D), and oviduct (E), but not in ovary (E) or uterus (F). Arrow indicates the infundibulum of the oviduct that was weakly labeled (E). No Spdef expression was detected by in situ hybridization in heart, thymus, thyroid, esophagus, spinal cord, brain, bladder, testes or epididymus (not shown). u, uterus. Scale bars: 200 μm.

FIG. 23 shows specificity of anti-sense probe for detection of Spdef mRNA. In situ hybridization was performed on sections of mouse lungs using anti-sense (A, C) and sense (B, D) probes for Spdef mRNA. While signal for Spdef mRNA was detected with anti-sense probe in lung from PN10 (A) and trachea from E17.5 (C), no hybridization was observed with sense probe in comparable tissue sections (B, D). Scale bars: 200 μm.

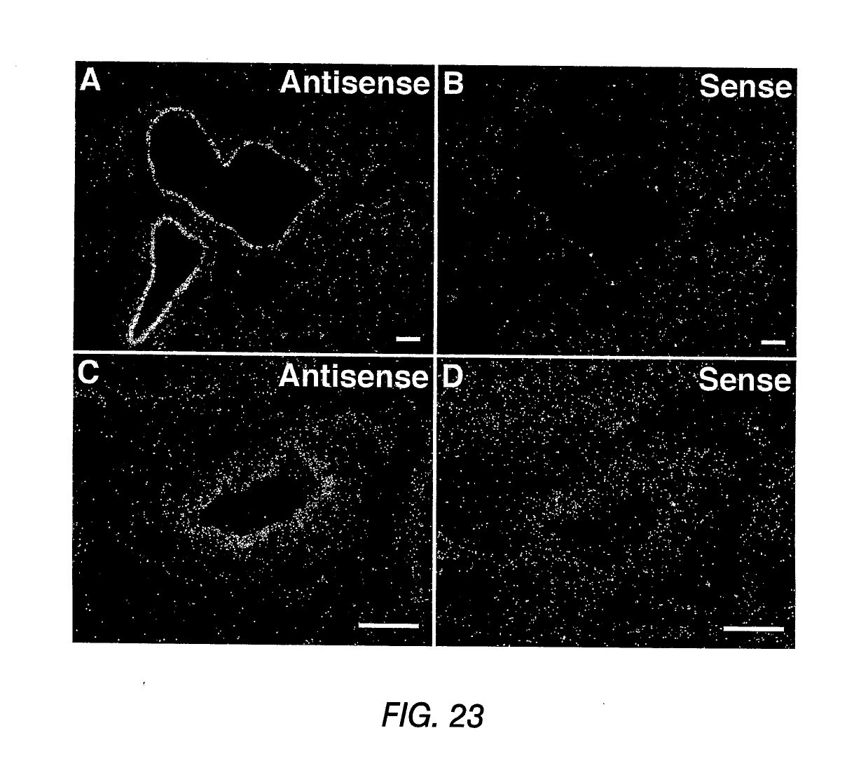

FIG. 24 demonstrates the specificity of Spdef polyclonal antibody. (A) Immunoblot analysis was performed on lysates of HeLa cells transfected with Spdef plasmid and adult mouse lung (mLu) and trachea (mTra) using guinea pig polyclonal antibody (described in Materials and Methods). A single band of approximately 37 kDa (arrowheads) was detected in HeLa cells transfected with Spdef cDNA, in tracheal lysates, but not in lysates of normal HeLa cells or lung parenchyma. (B) Immunocytochemistry was performed on HeLa cells transfected with Spdef expression plasmid using Spdef antibody. Nuclear staining was detected in transfected HeLa cells, but not in untransfected HeLa cells (arrow). DAPI was used to counterstain nuclei.

FIG. 25 demonstrates that Spdef was expressed in the epithelium of prostate, oviduct, and intestine. Immunohistochemistry for Spdef was performed on sections of adult mouse tissues. Nuclear staining was detected in epithelium of the seminal vesicles and coagulating glands (A), epithelial cells lining oviduct (B), and subsets of epithelial cells in the colon (C). Scale bars: A-D, 50 μm.

FIG. 26 shows that Spdef activates the Foxj1 promoter in vitro. (A) A schematic diagram of the promoter region of Foxj1. The locations of the putative ETS binding sites (GGAA/T) are indicated. HeLa cells were co-transfected with the increasing doses of plasmids expressing Spdef and Foxj1 reporter plasmids (B). All of the reporter plasmids were activated by Spdef in a dose dependent manner (0.01, 0.02, and 0.05 pM). Mutations of the two putative ETS binding sites in 0.25 Foxj1-luc plasmid (m1 and m2) did not affect the activation of the reporter by Spdef (C). Control (con) was the co-transfection of the reporter plasmid and the empty expression plasmid. Transfections were performed in triplicate and repeated three times with similar results. Values are mean±SD.

FIG. 27 shows that expression of Spdef did not induce expression of proinflammatory mediators. RT-PCR for Spdef, IL-13, IL-4, IL-6, TGF-α, Heparin Binding (HB)-EGF mRNAs was performed in lungs from rCCSP-rtTA/TRE2-Spdef mice treated with or without doxycycline. Spdef mRNA was increased more than three fold when the mice were treated with doxycycline. Levels of the cytokines and growth factors associated with goblet cell hyperplasia were not altered in the transgenic mice treated with doxycycline, TGF-α, mRNA was not detected (ND). Values are mean±SD, n=6 each group.

### DETAILED DESCRIPTION OF THE PREFERRED EMBODIMENT

Several proteins have been found to be involved in respiratory repair pathways. Among these are Sox17, Spdef, and other proteins. The expression, upregulation, and regulatory activities of these proteins, as well as their use for therapeutic treatments, is described herein.

The respiratory epithelium is lined by diverse cell types that vary along the cephalo-caudal axis during development and following acute or chronic injury. Ciliated epithelial cells can serve as a source of progenitor cells capable of rapid squamous differentiation, proliferation, and redifferentiation to restore the complex airway epithelium following acute bronchiolar cell injury. The ability of ciliated cells to proliferate and differentiate into multiple cell types of airway epithelium, and to self-renew satisfy the properties ascribed to tissue stem/progenitor cells.

The present invention relates to the finding that ciliated bronchiolar epithelial cells, previously considered to be terminally differentiated, can rapidly undergo squamous cell metaplasia, proliferate, and re-differentiate to restore ciliated and non-ciliated cell types lining the bronchioles after injury.

Further, Sox17 expression, which is normally restricted to ciliated cells in the adult lung, was found to be enhanced during regeneration of the bronchiolar epithelium following naphthalene injury and during compensatory lung growth following unilateral pneumonectomy. Dynamic changes in immunostaining of transcription factors, which play important roles in lung morphogenesis, accompanied the regeneration process. In transgenic mice, Sox17 was sufficient to induce ectopic differentiation of multiple cell types, as well as hyperplasia of both ciliated and non-ciliated cells in the peripheral lung, and to induce ciliated cell differentiation in the fetal lung. These findings demonstrate that ciliated epithelial cells serve as a source of multipotent progenitor cells, and that Sox17 regulates ciliated cell differentiation and influences progenitor cell behavior in the bronchiolar epithelium. Accordingly, in some embodiments of the present invention, administration of Sox17, or agents that induce expression of Sox17, can be used to assist in the repair process of lungs damaged by various pulmonary diseases.

Following injury, stem/progenitor cells proliferate and differentiate to replenish cell types and restore organ function. Ciliated precursors can undergo rapid transdifferentiation to produce squamous cells that serve as progenitors from which the diverse cell populations characteristic of the conducting airways are derived during repair of the respiratory epithelium. Molecular mechanisms underlying this repair process involve mediating cell signaling and transcriptional programs regulating cell survival, proliferation, and differentiation. Among the protein factors known to regulate transcriptional activity of progenitor cells, Sox proteins, I3-catenin, and Stat-3 have been identified as important factors in several tissues. The research findings described herein demonstrate that dynamic changes in the expression of Sox17, β-catenin, and Stat-3 occur in the respiratory epithelium following severe airway injury. These changes precede dynamic changes in transcription factors and cell differentiation markers that accompany the repair process. An understanding of this important pathway of pulmonary repair can be utilized to identify and prepare therapeutic formulations useful in treating and repairing many types of lung injury and disease.

The identification of respiratory epithelial progenitor cells and the genetic programs controlling their behavior during the repair process are of interest in the study of many types of pulmonary diseases, such as acute and chronic pulmonary disorders. The term “pulmonary disease” can include a large number of diseases, environmental factors, and genetic factors in which the lung function is impaired. The impairment can be chronic, intermittent, or acute. Organisms such as bacteria, fungi, and viruses can cause lung disease. Additionally, other causes, such as smoking, inhalation of chemicals, or genetic factors, can contribute to lung diseases. Several types of lung injury can also result in lung impairment. Examples of pulmonary diseases include but are not limited to bronchiectasis, bronchitis, bronchopulmonary dysplasia, interstitial lung disease, occupational lung disease, emphysema, cystic fibrosis, acute respiratory distress syndrome (ARDS), asthma, chronic bronchitis, COPD (chronic obstructive pulmonary disease), emphysema, interstitial lung disease, pulmonary fibrosis, sarcoidosis, asbestosis, aspergilloma, aspergillosis, pneumonia, pulmonary fibrosis, pulmonary tuberculosis, rheumatoid lung disease, and the like.

The maintenance of pulmonary homeostasis requires the capacity for rapid repair of the epithelial surfaces after various types of injury. Several models have been proposed in order to better understand the ways in which the lung is capable of repair. Extrapulmonary bone, marrow-derived cells migrate to the lung, contributing to the repair of the respiratory epithelium following injury (Krause et al., Cell, 105:369-377 (2001), which is incorporated by reference herein in its entirety). However, models in which rare progenitor cells account for the rapid and extensive repair of the lung are not compatible with the observed short period of proliferation and rapid restoration of epithelial surfaces that are observed after catastrophic injury caused infection or toxicants. The lung repair capacity is more consistent with a model in which relatively abundant or multiple progenitor cells participate in regeneration of the respiratory epithelium. In vitro studies support the concept that both basal and non-ciliated (Clara) respiratory epithelial cells in the conducting airways, and type II cells in the alveoli, maintain proliferative capacity (Ford et al., Exp. Cell. Res., 198:69-77 (1992); Van Winkle et al., Am. J Respir. Cell. Mol. Biol., 15:1-8 (1996); Rice et al., Am. J. Physiol., 283:L256-L264 (2002), each of which is incorporated by reference herein in its entirety). Indeed, widespread proliferation of type II epithelial cells accompanies growth of the remaining lung following unilateral pneumonectomy (Kaza et al., Circulation, 106:1120-1124 (2002), which is incorporated by reference herein in its entirety). Injury induced by hyperoxia or SO2 causes proliferation of type H and nonciliated airway epithelial cells (Tryka et al., Am. Rev. Respir. Dis., 133:1055-1059 (1986); Adamson et al., Lab. Invest., 30:35-42 (1974), each of which is incorporated by reference herein in its entirety).

Repair of the respiratory epithelium while maintaining lung function requires a rapid cellular response to restore permeability barriers, to cause proliferative responses of presumed progenitor cells, and to initiate redifferentiation of the diverse epithelial cell types characteristic of the normal lung. Many of the concepts regarding lung cell differentiation and proliferation are derived from developmental studies. Signaling via various growth factors and cytokines have been implicated in both lung morphogenesis and repair (Demayo et al., Am. J. Physiol., 283:L510-L517 (2002); Shannon et al., Annu. Rev. Physiol., 66:625-645 (2004) for review, each of which is incorporated by reference herein in its entirety). Transcription factors, such as TTF-1, Fox family members (including Foxa1, Foxa2, and Foxj1), GATA-6, and β-influence genetic programs critical for lung morphogenesis, differentiation, and pulmonary homeostasis (Costa et al., Am. J. Physiol., 280:L823-L838 (2001), which is incorporated by reference herein in its entirety). As described herein, the molecular mechanisms regulating differentiation and proliferation during development can also be involved in lung regeneration following injury or resection.

The Sox17 protein is a member of the Sry-related HMG box family of transcription factors. Targeted deletion of Sox17 in mouse causes severe defects in endoderm development and early embryonic lethality (Kanai-Azuma et al., Development, 129:2367-2379 (2002), which is incorporated by reference herein in its entirety). The role of Sox17 in development and function of adult organs remains unknown. Sox17 acts as both transcriptional activator and repressor. During early endoderm development, Sox17 physically interacts with β-catenin and synergistically induces expression of genes expressed selectively in the endoderm, while repressing transcriptional activity of TCF/β-catenin complex (Sinner et al., Development, 131:3069-3080 (2004); Zoro et al., 1999, each of which is incorporated by reference herein in its entirety). Activity and specificity of Sox17 on its target genes can be determined by its interactions with proteins, as is the case for other Sox proteins (Kamachi et al., Trends Genet., 16:182-187 (2000); Wilson and Koopman, Curr Opin Genet Dev., 12:441-446 (2002); each of which is incorporated by reference herein in its entirety).

The transcription factor Stat-3 is activated by several signaling molecules, including IL-6, IL 11, SCF, LIF, and others, to regulate cell survival, proliferation, migration, and inflammation. Administration of IL-6 protected the lung from injury by oxygen. Likewise, Stat-3 was activated following LPS induced lung injury in the epithelial cells of the conducting airways. Consistent with its role in cytoprotection, Cre-mediated conditional deletion of Stat-3 enhanced epithelial cell injury, and decreased surfactant production during hyperoxic injury, demonstrating its requirement for cell survival and differentiation to maintain lung function (Hokuto, J Clin Invest., 113:28-37 (2004), which is incorporated by reference herein in its entirety). Stat-3 has also been found to be required for survival during oxygen induced lung injury.

A mouse model was used to investigate whether Sox17, β-catenin, and Stat-3 are expressed in various stages of developing lung tissue. Transgenic mice were prepared by oocyte injection of a plasmid construct having the cDNA of a full length Sox17 sequence or a truncated Sox17 sequence as described in Example 1. The immunohistochemical methods and in situ hybridization methods used are described in Example 2. Mouse lung tissue was also examined by electron microscopy, as described in Example 3.

The mouse model was also used to examine whether Sox17, β-catenin, and Stat-3 are involved in the process of pulmonary repair after tissue damage. To produce damaged pulmonary tissue for the subsequent experiments on the repair process, the mice were treated with an intraperitoneal injection of naphthalene to denude bronchioles, as described in Example 1. The expression of Sox17 and other proteins was studied during the repair process.

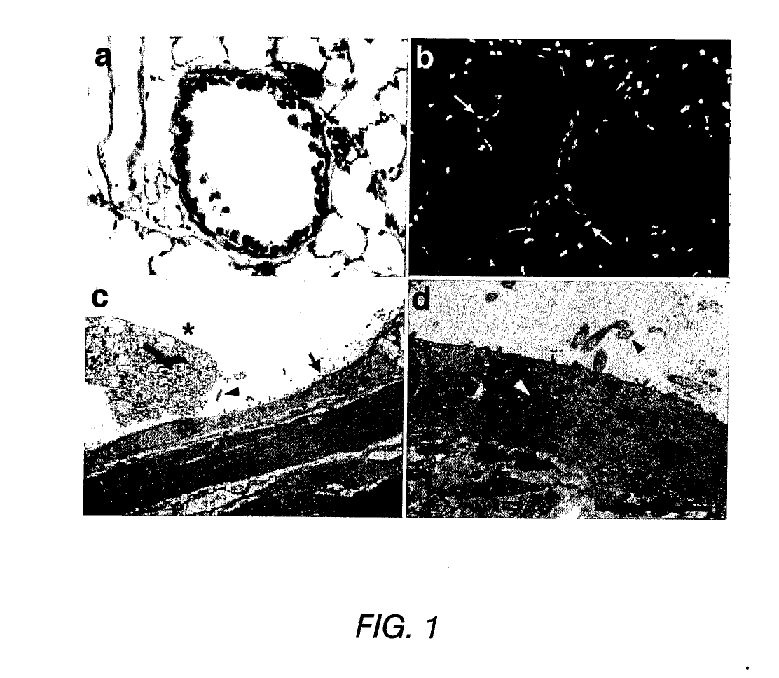

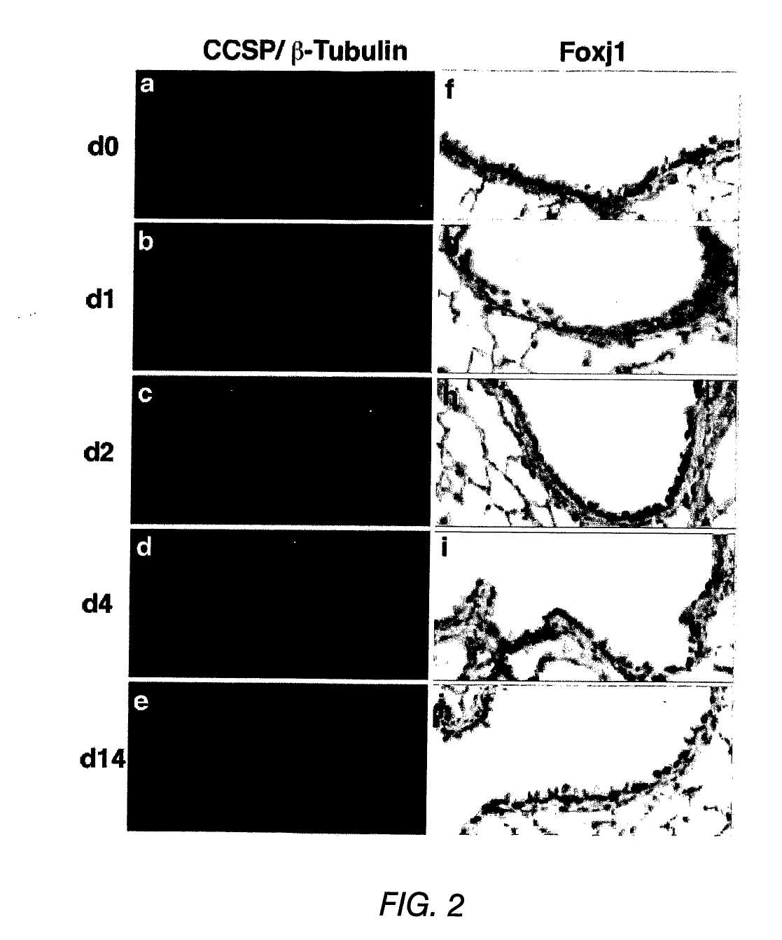

In order to identify progenitor cells that can mediate the repair of the bronchiolar epithelium, an intraperitoneal naphthalene injection was used to induce bronchiolar injury in mice, as detailed in Example 1. Naphthalene is concentrated in non-ciliated bronchiolar epithelial cells (Clara cells) that are enriched in P450 enzymes (CYP 2F2) that generate toxic metabolites resulting in bronchiolar cell injury (Mahvi et al., Am. J. Pathol., 86:558-572 (1977), which is incorporated by reference herein in its entirety). After naphthalene injury to the adult lung, virtually all squamous cells lining the “denuded” bronchioles were fluorescent, indicating their origin from a subset of endodermally-derived bronchiolar epithelial cells (FIG. 1b). Ciliated cells lining the conducting airways were identified as a source of progenitor cells that undergo rapid squamous metaplasia following acute lung injury, as discussed in Example 5 and as shown in FIG. 2, demonstrating that after naphthalene injury, ciliated cells can undergo squamous metaplasia and redifferentiate into both ciliated and non-ciliated cell types.

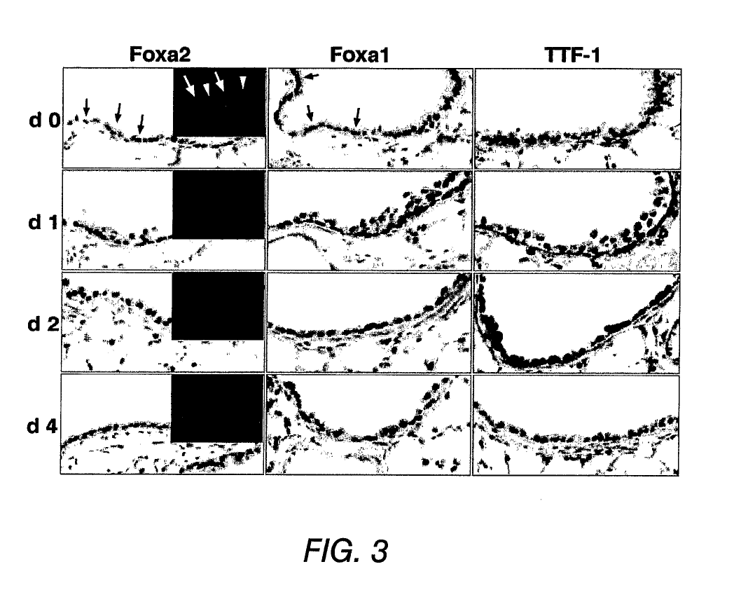

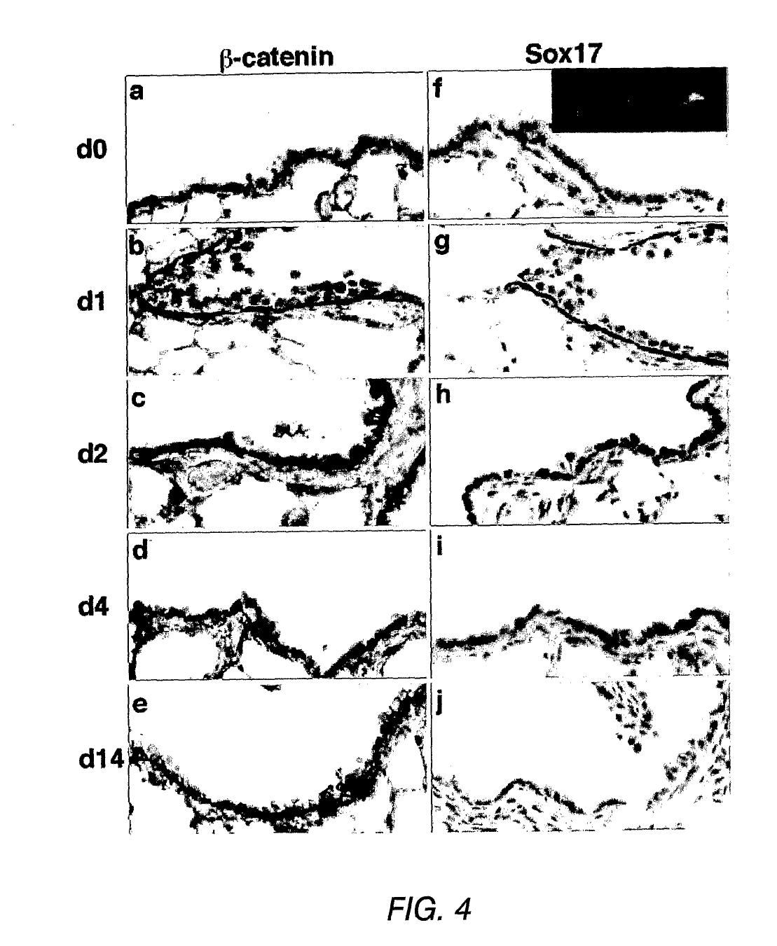

The expression of transcription factors known to be critical for fetal lung morphogenesis was assessed during recovery from naphthalene injury, in order to determine whether these factors are also involved in repair of the adult lung after an injury. The results are described in Example 6 and shown in FIGS. 3 and 4. In the normal lung, Foxa1, Foxa2, and Foxj1 were expressed selectively in ciliated epithelial cells lining the bronchioles while TTF-1 staining was more widespread (FIG. 3). Squamous and transitional cuboidal cells all stained intensely for Foxa1, Foxa2, and Foxj1 24-48 hours after the injury and their expression became increasingly restricted to subsets of ciliated cells during redifferentiation.

The protein β-catenin is known to be involved in lung branching morphogenesis, differentiation of respiratory epithelium, intracellular signaling, and can also be associated with the transmembrane adhesion protein cadherin. Conditional deletion of β-catenin in lung epithelial cells in the fetal lung perturbed branching morphogenesis, restricting formation of the peripheral lung and enhancing formation of the conducting airways (Mucenski, J. Biol. Chem., 278:40231-40238 (2003), which is incorporated by reference herein in its entirety). Expression of an activated β-catenin-Lef1 fusion protein in lung epithelial cells of fetal lung resulted in defects in branching morphogenesis and differentiation of respiratory epithelial cells, as well as expression of multiple genes characteristic of intestinal epithelial secretory cell types (Okubo and Hogan, J. Biol., 3:11 (2004), which is incorporated by reference herein in its entirety). These loss- and gain-of-function studies suggest that precise regulation of Wnt/β-catenin signaling is necessary for fate determination of respiratory epithelial progenitor cells. β-catenin interacts with Tcf/LefHMG box transcription factors to regulate the expression of downstream target genes. Multiple Sox proteins, including Sox17, Sox3, Sox7 and Sox9, also interact with β-catenin and can modulate its transcriptional activity (Zorn, Mol Cell., 4:487-498 (1999); Takash, Nucleic Acids Res., 29:4274-4283 (2001); Zhang, Development, 130:5609-5624 (2003); Sinner, Development, 131:3069-3080 (2004); each of which is incorporated by reference herein in its entirety).

Accordingly, the expression pattern of β-catenin was examined during repair of the bronchiolar epithelium (FIG. 4). Interestingly, the subcellular location of β-catenin was altered after lung injury. β-catenin staining in normal adult lungs is normally cytosolic and membrane-associated and rarely observed in nuclei of airway epithelial cells (FIG. 4a). Twenty-four to 48 hours after injury, however, nuclear and cytoplasmic staining for β-catenin was markedly increased in the squamous and cuboidal cells lining the bronchioles (FIGS. 4b, c). Four days after injury and afterward, β-catenin staining decreased and was restored to the pattern seen in the normal adult lung (FIGS. 4d, e).

Transcription factors specific to ciliated bronchiolar cells can play an important role in the repair process. Because Foxa1 and Foxa2 are regulated by interaction of Sox17 and β-catenin in early Xenopus endoderm, the cellular localization of Sox17 was determined in the adult mouse lung using immunohistochemical methods (Example 7). Sox17 was selectively expressed in ciliated respiratory epithelial cells following injury. At 24-48 hours after naphthalene-induced injury, intense Sox17 staining was observed in all of the squamous and cuboidal cells lining the bronchioles (FIGS. 4g, h). The expression pattern of Sox17 suggests that Sox17 can regulate expression of genes in ciliated cells or the progenitor cells derived from them, during regeneration of the bronchiolar epithelium.

In addition to the naphthalene-induced airway damage model, another model of lung damage, a unilateral pneumonectomy, was utilized to determine whether similar transcriptional pathways occur during the repair process (Example 8). It has been previously shown that compensatory lung growth occurs in conducting airways as well as lung parenchyma following unilateral pneumonectomy (Nakajima et al., Pediatr. Surg. Int., 13:341-345 (1998); Laros et al., J. Thorac. Cardiovsc. Surg., 93:570-576 (1987), each of which is incorporated by reference herein in its entirety). Thus, the regrowth of bronchiolar epithelium was examined after a pneumonectomy in order to determine whether it utilizes the same transcriptional network that occurs in ciliated cells during repair after naphthalene injury.

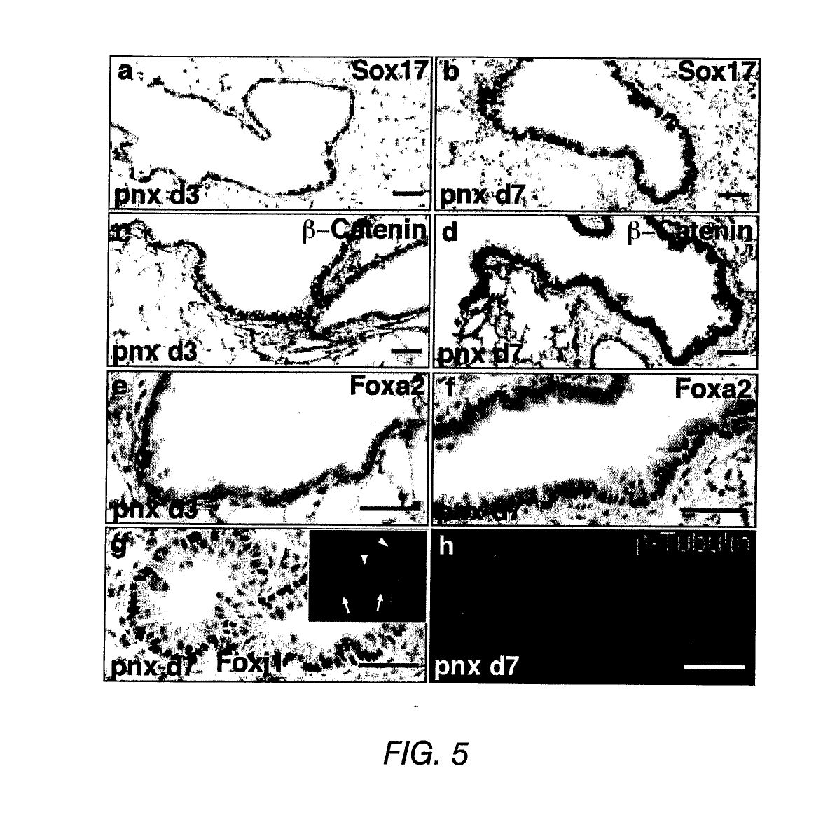

The results show that Sox17, β-catenin; Foxa2, and Foxj1 were expressed during Lung Regeneration Following Unilateral Pneumonectomy (FIG. 5; Example 8). Marked hyperplasia of both peripheral (alveolar) and bronchiolar epithelia was observed following pneumonectomy, being most evident 7 days after surgery and decreased by 14 days after surgery (FIG. 5). Thus, ciliated cells were capable of regaining proliferative capacity after pneumonectomy.

The results of Example 8 demonstrate that transcriptional programs induced in ciliated bronchiolar cells during injury were also activated during regeneration of the lung following unilateral pneumonectomy. Example 8 further demonstrates that the ciliated cells regained proliferative capacity. These findings challenge previous models in which rare subsets of respiratory epithelial cells migrate from specialized niches to repair the lung after injury, and are not consistent with a significant role for extrapulmonary cells e.g. bone marrow-derived cells or mesenchymal stem cells, in repair of respiratory epithelium following injury (Reynolds et al., Am. J. Pathol., 156:269-278 (2000); Hong et al., Am. J. Respir. Cell Mol. Biol., 24:671-681 (2001); Borthwick et al., Am. J. Respir. Cell Mol. Biol., 24:662-670 (2001); Krause et al., Cell, 105:369-377 (2001); Ortiz et al., Proc. Natl. Acad. Sci. USA, 100:8407-8411 (2003), each of which is incorporated by reference herein in its entirety).

These results also challenge the view that ciliated cells represent a subset of terminally differentiated airway epithelial cells. The concept that a relatively abundant subset of progenitor cells can rapidly spread, proliferate and redifferentiate to regenerate a complex airway epithelium, provides a basis for the rapid repair of the lung following infection, exposure to toxicants and after lung resection.

The rapid and widespread reprogramming of bronchiolar epithelial cells was accompanied by dynamic changes in Sox17 and other transcription factors known to play important roles in lung morphogenesis and cell differentiation, including Foxa1, Foxa2, Foxj1, and TTF-1 (Wan et al., Development, 131:953-964 (2004); Chen et al., J. Clin. Invest., 102:1077-1082 (1998); Brody et al., Am. J. Respir. Cell. Mol. Biol., 23:45-51 (2000); Wan et al., Proc. Natl. Acad. Sci. USA, 101:14449-14454 (2004); Wan et al., J. Biol. Chem., In press (2005); Kimura et al., Genes Dev., 10:60-69 (1996), each of which is incorporated by reference herein in its entirety). In naphthalene induced injury, selective loss of non ciliated cells occurs without apparent injury to or proliferation of alveolar cells. Activation of this transcriptional program was observed in bronchioles during both repair following injury and compensatory lung growth following unilateral pneumonectomy.

In the pneumonectomy model (Example 8), marked hyperplasia and proliferation occurs in both airways and the alveoli, involving multiple epithelial and non-epithelial cell types, including ciliated cells. Nevertheless, dynamic changes in the same transcription factors were associated with regeneration of the bronchiolar epithelium in both models. These observations support the concept that the generation of bronchiolar epithelium, at least in part, recapitulates transcriptional programs that coordinate respiratory epithelial cell differentiation during normal lung development. Since multiple cell types proliferate and differentiate following injury or during compensatory growth, it is anticipated that distinct transcriptional programs will influence the processes in diverse cell types.

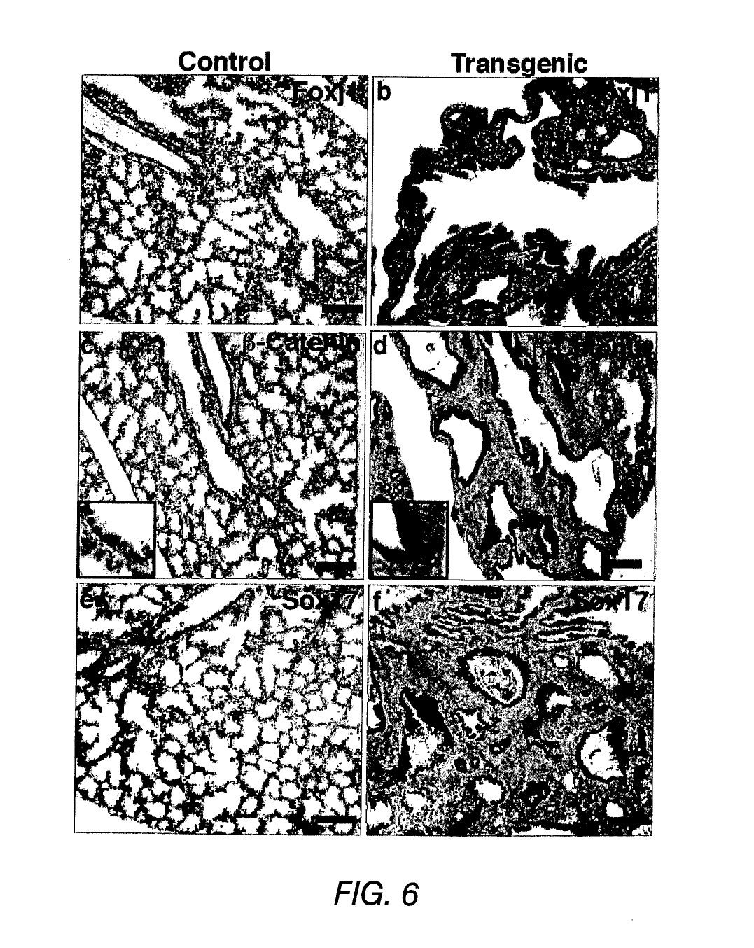

The finding that Sox17 was induced after bronchiolar injury and during regrowth following pneumonectomy led to the possibility that Sox17 can play a role in specification of ciliated cells. To test this, Sox17 was expressed in respiratory epithelium of fetal mice under conditional control of the SP-C promoter (Peri et al., Transgenic Res., 11:21-29 (2002), which is incorporated by reference herein in its entirety) (Example 9). Sox17 disrupted branching morphogenesis and altered differentiation of epithelial cells lining the lung tubules at E 18, producing a hyperplastic bronchiolar epithelium (FIGS. 6b-f). Sox17 alone was sufficient to induce a ciliated cell phenotype during fetal lung morphogenesis. Further, Sox17 was capable of activating the Foxj1 promoter, which is critical for ciliogenesis (Chen et al., J. Clin. Invest., 102:1077-1082 (1998); Brody et al., Am. J. Respir. Cell. Mol. Biol., 23:45-51 (2000); You et al., Am. J. Physiol., 286:L650-L657 (2004), each of which is incorporated by reference herein in its entirety).

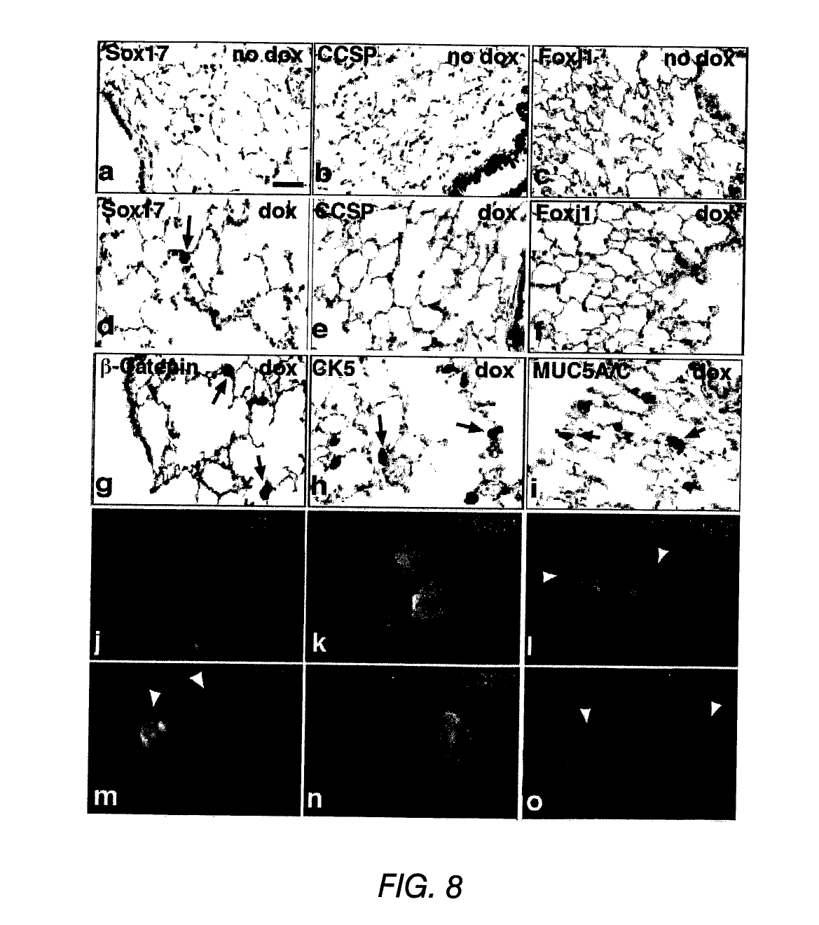

The finding of the widespread expression of Sox17 in association with the process of transdifferentiation of squamous progenitor cells in the bronchioles during repair of the adult lung, led to the possibility that Sox17 can influence progenitor cell behavior in vivo. To test this, conditional expression of Sox17 in respiratory epithelial cells in adult mice under control of the CCSP promoter was performed, as described in Example 10 and shown in FIG. 8.

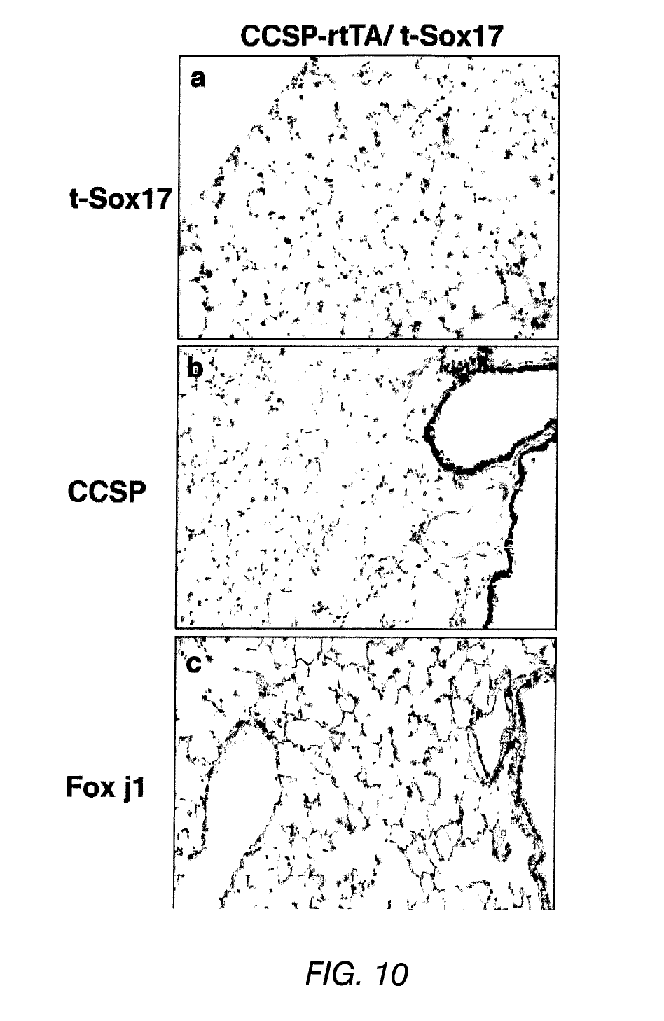

Interestingly, a truncated form of Sox17 (tSox17), that lacks most of the HMG box and does not bind to DNA, did not cause ectopic airway cell differentiation in the lung periphery (FIG. 10), and did not alter lung histology. Thus, expression of Sox17 in vivo increased nuclear β-catenin staining and influenced respiratory epithelial cell differentiation, inducing ectopic clusters of epithelial cells expressing multiple markers specific for conducting airway epithelial cells but in the alveoli of the adult lung.

As described herein, both Sox17 and β-catenin were co-expressed in the squamous and cuboidal progenitor cells during repair or regeneration of the respiratory epithelium. Expression of Sox17 in transgenic mice in vivo coincided with increased β-catenin staining and ectopic, widespread expression of Foxj1. Sox17 also activated the Foxj1 promoter, which is required for ciliogenesis (Chen et al., J Clin. Invest., 102:1077-1082 (1998); Brody et al., Am. J Respir. Cell. Mol. Biol., 23:45-51 (2000); You et al., Am. J. Physiol., 286:L650-L657 (2004), each of which is incorporated by reference herein in its entirety). Sox17 and β-catenin are known to interact to regulate subsets of genes in the early endoderm, including Foxa1 and Foxa2 (Sinner et al., Development, 131:3069-3080 (2004), which is incorporated by reference herein in its entirety). Foxa1, Foxa2, and Foxj1 were also dynamically regulated following injury and restoration of the bronchiolar epithelium. Foxa1, Foxa2, and Foxj1, were co-expressed in ciliated epithelial cells of the developing and mature lung. These Fox transcription factors influence gene expression and epithelial cell differentiation in the lung (Wan et al., Development, 131:953-964 (2004); Chen et al., J. Clin. Invest., 102:1077-1082 (1998); Brody et al., Am. J. Respir. Cell. Mol. Biol., 23:45-51 (2000); Wan et al., Proc. Natl. Acad. Sci. USA, 101:14449-14454 (2004); Wan et al., J. Biol. Chem., In press (2005), each of which is incorporated by reference herein in its entirety).

The extent and intensity of Sox17 expression increased as undifferentiated squamous progenitor cells covered the injured bronchioles prior to proliferation. The progenitor cells became cuboidal and proliferated 2-3 days after injury, likely representing a so-called rapid amplifying pool of cells. Subsequently, restriction of Sox17 expression occurred as the progenitor cells differentiated from cuboidal to columnar cell types. Variations in the levels of Sox17 were also associated with distinct patterns of expression of various epithelial cell markers in the CCSP-driven Sox17 transgene mice, suggesting that the level of Sox17 influences cell type specific gene expression. These findings also support the concept that Sox17 regulates progenitor behavior of bronchiolar epithelial cells. The observation that high levels of Sox17 expression induced widespread ciliated cell differentiation in the fetal lung in vivo provides further evidence that Sox17 influences respiratory epithelial differentiation. Sox17 activated the Foxj1 gene promoter, providing a mechanism by which Sox17 can influence ciliated cell differentiation.

As demonstrated herein, the expression of Sox17 in the adult mouse lung in vivo caused trans differentiation of alveolar respiratory epithelial cells into distinct subsets of epithelial cells expressing multiple proximal airway markers, causing hyperplastic foci of epithelial cells that were relatively undifferentiated, expressing proximal airway markers including Foxj1. The hyperplastic, multicellular lesions caused by Sox17 in the adult lung also support a potential role of Sox17 in the pathogenesis of metaplasia and turmorigenesis in the lung. Thus, following injury, Sox17 can influence progenitor cell behavior through reprogramming of transcription to direct the re-differentiation of the progenitor cells into multiple airway epithelial cell types.

As demonstrated herein, ciliated cells, generally considered a fully differentiated lung epithelial cell type, can undergo rapid squamous metaplasia associated with enhanced expression of Sox17, β-catenin, and other transcription factors influencing epithelial cell differentiation. Cuboidal cells derived from the squamous progenitor cells proliferate and redifferentiate to restore the heterogeneous cells lining the normal respiratory epithelium. Dynamic changes in expression of Sox17, Foxa2, Foxa1, and Foxj1 accompanied the repair process as the progenitors restore columnar cells lining the bronchioles. Sox17 was sufficient to induce, at least in part, ciliated and progenitor cell behavior in the fetal and adult lung in vivo and to induce expression of Foxj1 in vitro.

Further, in some embodiments of the present invention, ciliated cells can be a source of progenitor cells that can be specific targets for correction of acquired and hereditary diseases of the lung. The ability of Sox17 to cause epithelial metaplasia and hyperplasia in the adult lung supports the concept that Sox17 plays a role in both repair and tumorigenesis in the respiratory epithelium.

Sox17 was found to be sufficient to specify the fate of respiratory epithelial cells toward proximal airway lineages during lung morphogenesis and in adults. In some embodiments of the invention, the finding of the important involvement of Sox17 in lung morphogenesis and repair makes the modulation of Sox17 levels a suitable target for pharmaceutical compositions to treat lung injuries.

β-catenin

During the repair process, β-catenin acts downstream of Sox17 to regulate differentiation of the airway epithelial progenitor cells during repair. Increased expression of Sox17 in the airway epithelial progenitor cells following injury was accompanied by increased nuclear β-catenin staining. Induction of Sox17 and transition of ciliated cells to squamous progenitor cells occurred in the absence of β-catenin, but subsequent epithelial cell differentiation was blocked. These findings suggest that β-catenin is required for restoration of the complex airway epithelium following injury, but is not necessary for reduction of Sox17 or for squamous cell differentiation of the progenitor cells. This important role of β-catenin in cell specification is consistent with its role in proximal-peripheral epithelial cell differentiation during lung morphogenesis as well as during repair or wound healing and tissue regeneration in various animal and cell models. Increased nuclear β-catenin was detected in hypoplastic and metaplastic lesions associated with idiopathic pulmonary fibrosis.

As shown herein, Sox17 and nuclear β-catenin were coregulated during repair of the respiratory epithelium. The expression of Sox17 can enhance β-catenin staining in vivo. Therefore, in some embodiments of the invention, high levels of Sox17 induce nuclear β-catenin during transdifferentiation following injury, which in turn plays a critical role in subsequent differentiation of the squamous progenitor cells.

Sox17 and β-catenin were found to directly interact and regulate a subset of endodermal genes in Xenopus (Sinner et al., Development, 131, 3069-3080 (2004), which is incorporated by reference herein in its entirety). Sinner et al. further suggested that Sox17 interacts with β-catenin to activate the transcription of its target genes in the early endoderm, including Foxa1 and Foxa2. Foxa1 and Foxa2 play important and complementary roles in differentiation of the respiratory epithelium (Wan, Development, 131:953-964 (2004); Wan, Proc. Natl. Acad. Sci. USA, 101:14449-14454 (2004), each of which is incorporated by reference herein in its entirety) and directly regulate transcriptional targets including CCSP and surfactant proteins A, B, C, D, that are differentiation markers of the respiratory epithelium. While Sox17 expression was induced following injury, it preferably acts with β-catenin in restoring differentiation of airway epithelial cell types.

The results described herein provide cellular and molecular evidence that ciliated cells, generally considered a terminally differentiated lung epithelium cell type, can undergo rapid squamous metaplasia. These changes are associated with enhanced expression of Sox17, Stat-3, and β-catenin. These squamous progenitor cells both proliferate and differentiate to restore the heterogeneous, differentiated cells lining the normal respiratory epithelium. A differentiation program involving β-catenin and associated with dynamic expression of Stat-3, Foxa2, Foxj1, and TTF-1 can accompany the repair process. Following acute injury, injured airway epithelium relies upon existing differentiated cell types that rapidly spread and regain proliferative capacity and differentiate repair of the respiratory epithelium during repair. Thus, as demonstrated herein, ciliated cells can act as potential progenitor cells.

Damaged respiratory epithelium can be repaired by introduction of genetic material to correct acquired and genetic diseases affecting the lung. Furthermore, the finding that Sox17 is sufficient to activate airway epithelial cell progenitor cell behavior provides potential therapeutic strategies to enhance repair of the lung. Accordingly, methods of treating lung injuries or lung diseases using Sox17 protein or a nucleic acid encoding it, are disclosed herein. Example 11 describes the preparation of a composition comprising a Sox17 nucleic acid. Examples 12-15 demonstrate the use of a Sox17 or Spdef nucleic acid to treat pulmonary injuries or pulmonary diseases.

### The Protein Spdef Activates Sox17 and can Act as an Agent to Upregulate Sox17

The Spdef protein (SAM pointed domain containing ets transcription factor), also termed prostate-specific Ets (Pse) is a member of the Ets family of transcription factors. Spdef was identified in a subset of conducting airway and in ciliated epithelial cells in the fetal and,postnatal lungs, respectively. Spdef activated expression of several respiratory epithelial cell target genes, including Sox17, and Foxj1 that are also selectively expressed in ciliated cells in the adult lung. Expression of Spdef, Sox17, and Foxj1 precede differentiation of the bronchiolar epithelium and ciliated cell differentiation in the fetal lung. Taken together, these findings support a transcriptional program wherein Spdef, Sox17, Foxj1, and Ttf-1 participate in differentiation of ciliated cells. Since Spdef is induced following lung injury, Spdef and its transcriptional targets can also play an important role in regeneration of the respiratory epithelium following injury. Examples 21 through 33 relate to aspects of the Spdef protein.

Increased expression of Spdef has been observed in the lungs of transgenic mice in which Ttf-1 gene was replaced with a phosphorylation mutant, supporting the concept that Spdef and Ttf-1 can participate in genetic programs regulating formation or function of the respiratory epithelium (deFelice et al., J. Biol. Chem. 278:35574-35583 (2003) which is incorporated by reference herein in its entirety). As shown herein in Example 24, Spdef expression in a subset of peripheral conducting airway cells in late gestation and in the adult mouse lung, and is co-expressed with Sox17, Foxj1, and β-tubulin in ciliated respiratory epithelial cells in the adult mouse lung. Spdef interacted with Ttf-1, and and acted in concert to enhance transcription of several target genes including Sox17 and Foxj1, as demonstrated in Examples 25 and 26. Thus, Spdef regulates transcription of a subset of genes controlling ciliated cell differentiation in the respiratory epithelium.

Because Spdef can regulate the transcription of Sox17 and other genes involved in pulmonary repair, such as Foxj1, Spdef protein, a fragment of Spdef, or a nucleic acid encoding it, can be used as an agent to upregulate Sox17 in an individual needing pulmonary repair. Two examples of the use of Spdef to advance pulmonary healing are shown in Examples 13 and 33. Further, since Spdef induces goblet cell differentiation, and is induced by injury and allergy induced hyperplasia, inhibition of the Sox17 or Spdef pathway can be useful in blocking hyperplasia and airway epithelial cell remodeling, as seen, for example, in asthma and COPD.

Because of the findings that Sox17 is an important player in the repair of lung tissue, the modulation of Sox17 levels as a treatment for pulmonary damage is contemplated herein. For the sake of clarity, embodiments of the present invention are described in detail in sections relating to pulmonary administration of agents that upregulate Sox17, Sox17-encoding nucleic acid, or the fragment or analog or derivative thereof, aerosol formulations, and methods for pulmonary treatment, repair, and prophylaxis. Likewise, embodiments of the invention are directed toward use of Sox17 in screening and selecting compounds suitable for treatment of lung injury and related conditions.

Spdef modulation can also be used as a treatment for pulmonary damage, and is described herein. Likewise, embodiments of the invention are directed toward use of Spdef in screening and selecting compounds suitable for treatment of lung injury and related conditions.

In some embodiments, Sox17-encoding nucleic acid or Spdef-encoding nucleic acid can be administered alone or in combination with β-catenin and/or Stat-3 proteins or nucleic acids to treat pulmonary diseases. As used herein the term “treat” or “treatment” refer to both therapeutic treatment and prophylactic or preventative measures, wherein the object is to prevent or slow down (lessen) an undesired physiological change or disorder, such as the development or spread of a lung injury or lung disease. The term “treat” also refers in some embodiments to the characterization of the type or severity of disease which can have ramifications for future prognosis, or need for specific treatments. For purposes of this invention, beneficial or desired clinical results can include in various embodiments, but are not limited to, alleviation of symptoms, diminishment of extent of a pulmonary disease or injury, stabilized (i.e., not worsening) state of a pulmonary disease or injury, delay or slowing of pulmonary disease progression, amelioration or palliation of the disease state, and remission (whether partial or total), whether detectable or undetectable. “Treatment” can also mean in some embodiments prolonging survival as compared to expected survival if not receiving treatment. Those in need of treatment include those already with the condition or disorder as well as those prone to have the condition or disorder or those in which the condition or disorder is to be prevented.

As used herein, the term “pulmonary administration” refers to administration of a formulation, for example a formulation of Sox17-encoding nucleic acid or Spdef-encoding nucleic acid through the lungs, in preferred embodiments by inhalation. As used herein, the term “inhalation” preferably refers to intake of air for example to the alveoli. In specific examples, intake can occur by self-administration of a formulation of the invention while inhaling, or by administration via a respirator, e.g., to a patient on a respirator. The term “inhalation” used with respect to a formulation of the invention is in preferred embodiments synonymous with “pulmonary administration.”

As used herein, the term “aerosol” refers preferably to suspension in the air. In particular, aerosol can refer to the particle formation of a composition of embodiments of the invention and its suspension in the air. According to embodiments of the present invention, an aerosol formulation can be a formulation comprising a Sox17-encoding nucleic acid, or Spdef-encoding nucleic acid, or the fragment or analog or derivative thereof that is suitable for aerosolization, for inhalation or pulmonary administration.

In some embodiments of the invention, a Sox17-encoding nucleic acid or Spdef-encoding nucleic acid is provided, which can be delivered to a host cell, for example by any of the above-mentioned aerosolization protocols or by any other suitable protocols. However, the nucleic acid can be delivered in a number of different forms. Nucleic acids can be delivered as naked DNA or within one or more vectors, the vectors including, but not limited to viral, plasmid, cosmid, liposome, and microparticles. Likewise, modified nucleic acids, such as, for example, peptide nucleic acids, can be employed in some embodiments.

A nucleic acid molecule encoding a Sox17 or Spdef polypeptide can be identified and isolated using standard methods, as described by Sambrook et al., Molecular Cloning: A Laboratory Manual; Cold Spring Harbor Press, New York (1989), which is incorporated by reference herein in its entirety. For example, polymerase chain reaction can be employed to isolate and clone Sox17 genes. Generally, sequence information from the ends of the region of interest or beyond is employed to design oligonucleotide primers. These primers preferably will be identical or similar in sequence to opposite or complimentary strands of the template to be amplified. PCR can be used to amplify specific RNA sequences, specific DNA sequences from total genomic DNA, and cDNA transcribed from total cellular RNA, bacteriophage or plasmid sequences and the like, to yield an amplification product. See also, Mullis et al., Cold Harbor Symp. Quant. Biol., 51:263 (1987); Erlich, ed., PCR Technology (Stockton Press, N.Y., 1989), each of which is incorporated by reference herein in its entirety. Alternatively, the Sox17 gene can be isolated from a library of the appropriate human or mammal, using a Sox17 probe.

Human Sox17 nucleic acid sequences are found in SEQ ID NOs. 1-4. An exemplary cDNA sequence is shown in SEQ ID NO: 1 (NCBI Accession No. NM_{—}022454). Exemplary Sox17 protein sequences are NCBI Accession Nos. BAB83867 (SEQ ID NO: 5), (SEQ ID NO: 6), and NP_{—}071899 (SEQ ID NO: 7).

An exemplary Spdef mRNA sequence is SEQ ID NO: 8 (NCBI Accession No, NM_{—}012391). An exemplary Spdef coding sequence is SEQ ID NO: 9 (NCBI Accession No. NM_{—}012391) An exemplary Spdef protein sequence is SEQ ID NO: 10 (NCBI Protein Accession No. NP_{—}036523). Additionally, the Sox2 protein is also co-expressed in pulmonary tissue, and can play a similar role. Accordingly, the Sox2 protein, its fragments, and nucleic acids encoding it, can also be useful for pulmonary treatment.

### Nucleic Acid Preparation and Administration Methods

Nucleic acid molecules encoding amino acid sequence variants of an active Sox17 or Spdef polypeptide can be prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of a Sox17 or Spdef gene.

To prepare expression cassettes or vectors for transfection, the nucleic acid sequence can be circular, linear, double-stranded, or single-stranded. The nucleic acid sequences can be transferred to microbial cells for amplification procedures, or can be transferred to eukaryotic cells, such as mammalian cells. The nucleic acid sequences can also be administered to a human. The method of preparation of the Sox17 or Spdef nucleic acid sequence can be varied, depending, for example, on its desired destination.

As used herein, the term “vector” is used in reference to nucleic acid molecules that transfer nucleic acid segment(s) to a cell. Vectors can be used, for example, to introduce foreign DNA into host cells where it can be replicated in large quantities. The term “vehicle” is sometimes used interchangeably with “vector.” Vectors, including “cloning vectors” can allow the insertion of nucleic acid fragments without the loss of the vector's capacity for self-replication. Vectors can be derived from viruses, plasmids or genetic elements from eukaryotic and/or prokaryotic organisms; vectors frequently comprise DNA segments from several sources. Expression cassettes or expression vectors for host cells ordinarily include an origin of replication, a promoter located upstream from the Sox17 coding sequence, a ribosome-binding site, a polyadenylation site, and a transcriptional termination sequence. Those of ordinary skill in the art will appreciate that some of the aforementioned sequences are not required for expression in certain hosts. In some embodiments of the present invention, an expression cassette is constructed so that a human Sox17 nucleic acid sequence is located in the cassette with at least one appropriate regulatory sequence, the positioning and orientation of the coding sequence with respect to the control sequence being such that the coding sequence is transcribed under the “control” of the control sequence.

The transfection process can be by any method known to those in the art for introducing polynucleotides into a host cell, including, for example, packaging the polynucleotide in a virus and transducing a host cell with the virus, and by direct uptake of the polynucleotide, such as by electroporation or particle bombardment. The Sox17 encoding nucleic acid may or may not be integrated (covalently linked) to the chromosomal DNA of the cell. Other methods for the introduction of nucleic acids into mammalian cells include, for example, dextran mediated transfection, calcium phosphate mediated transfection, polybrene mediated transfection, electroporation, protoplast fusion, encapsulation of the polynucleotides in liposomes, and direct microinjection of the nucleic acid into nuclei.

As used herein, the term “purified” does not require absolute purity; rather, it is intended as a relative definition. Purification of starting material or natural material to at least one order of magnitude, preferably two or three orders, and more preferably four or five orders of magnitude is expressly contemplated. The term “purified” is used herein to describe a preferred polypeptide or nucleic acid of the invention which has been separated from other compounds including, but not limited to, other nucleic acids, lipids, carbohydrates and other proteins.

The term “homologous” refers to an evaluation of the similarity between two sequences based on measurements of sequence identity adjusted for variables including gaps, insertions, frame shifts, conservative substitutions, and sequencing errors. Two nucleotide sequences or polypeptides are said to be “identical” if the sequence of nucleotides or amino acid residues, respectively, in the two sequences is the same when aligned for maximum correspondence as described herein. As used herein the term “homology” refers to comparisons between protein and/or nucleic acid sequences and is evaluated using any of the variety of sequence comparison algorithms and programs known in the art.

The term “substantially homologous,” when used herein with respect to a nucleotide sequence, refers to a nucleotide sequence corresponding to a reference nucleotide sequence, wherein the corresponding sequence encodes a polypeptide having substantially the same structure as the polypeptide encoded by the reference nucleotide sequence. In some embodiments, the substantially similar nucleotide sequence encodes the polypeptide encoded by the reference nucleotide sequence. In the context of the present invention, “substantially homologous” refers to nucleotide sequences having at least 50% sequence identity, or at least 60%, at least 70%, at least 80%, at least 85%, at least 90%, at least 95%, at least 96%, at least 97%, 98%, or at least 99% sequence identity compared to a reference sequence that encodes a protein having at least 50% identity, or at least 85%, at least 90%, at least 95%, at least 96%, at least 97%, 98%, or at least 99% sequence identity to a region of sequence of a reference protein.

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

In some embodiments of the present invention, a nucleic acid fragment of the full length Sox17 or Spdef can be administered. A Sox17 or Spdef nucleic acid fragment can be, for example, at least 20, 50, 100, 150, 200, 250, 300, 400, 450, or 480 or more nucleotides in length. Further, if desired, a chimeric nucleic acid molecule comprising at least a portion of the Sox17 or Spdef sequence, in combination with another sequence, can be used.

In some embodiments of the present invention, vectors or expression cassettes comprising the Sox17 or Spdef nucleic acid can be prepared for use in pulmonary administration. To prepare expression cassettes or vectors or other nucleic acids, the recombinant or preselected nucleic acid sequence or segment can be circular or linear, double-stranded or single-stranded. Expression cassettes or expression vectors for host cells ordinarily include an origin of replication, a promoter located upstream from the Sox17 or Spdef coding sequence, together with a ribosome binding site, a polyadenylation site, and a transcriptional termination sequence. Example 11 demonstrates the preparation of a Sox17 or Spdef encoding vector that can be used for pulmonary administration.

The amount of Sox17 or Spdef nucleic acid, or the fragment or analog or derivative thereof, that is used for treatment can vary based on several factors. An “effective amount” of a compound to be employed therapeutically will depend, for example, upon the therapeutic objectives, the route of administration, the type of compound employed, and the condition of the patient. Accordingly, it can be beneficial to titrate the dosage and modify the route of administration as required to obtain the optimal therapeutic effect. Typically, the clinician will administer the compound until a dosage is reached that achieves the desired effect. The progress of this therapy is easily monitored by conventional assays.

In some embodiments of the present invention, a nucleic acid comprising the Sox17 or Spdef coding region can be administered to the lungs for treatment of a pulmonary injury. The preparation of the nucleic acid encoding Sox17 or Spdef is within the skill of one with general knowledge of the art. Methods of preparing nucleic acids suitable for pulmonary delivery are described, for example, in U.S. Pat. No. 6,211,162 to Dale et al.; U.S. Pat. No. 6,921,527 to Platz; French et al., (1996) J. Aerosol Science, 27: 769-783; and Gonda, I., (1990) “Aerosols for Delivery of Therapeutic and Diagnostic Agents to the Respiratory Tract,” Critical Reviews in Therapeutic Drug Carrier Systems, 6: pp. 273-313 (1990), each of which is incorporated by reference herein in its entirety. Nucleic acid can be delivered, for example, using compacted DNA particles, plasmids, viral vectors, such as adenoviral vectors and lentiviral vectors. Additional examples of the use of viral vectors for pulmonary administration can be found, for example, in Zsengeller et al., Hum. Gene Ther. 9:2101-2109 (1998); Harrod et al., Hum Gene Ther. 9: 1885-1898 (1998); Jobe et al., Hum. Gene Ther. 7:697-704 (1996); and Otake et al., Hum. Gene Ther. 9:2207-2222 (1998), each of which is incorporated by reference herein in its entirety.

In some embodiments of the invention, the Sox17 or Spdef nucleic acid can be prepared as an aptamer. The use of nucleic acid aptamers can increase the stability of the nucleic acid in the cell. Preparation and use of nucleic acid aptamers for therapeutics is described, for example, in Pendergrast (2005) Jour. Biomol. Tech. 16:224-234, which is incorporated by reference herein in its entirety.

In some embodiments of the invention, the Sox17 or Spdef nucleic acid can be administered to the patient in the form of a liposomal composition. For example, Legace et al. (J. Microencapsulation, 8:53-61(1991), which is incorporated by reference herein in its entirety), describes the preparation of liposomes containing protonated/acidified nucleic acids, which are useful for pulmonary administration.

The formulation of Sox17 or Spdef nucleic acid, or the fragment or analog or derivative thereof, will be formulated, dosed, and administered in a fashion consistent with good medical practice. Factors for consideration include the level of the pulmonary disease or injury being treated, the clinical condition of the individual patient, the site of delivery of the formulation, the method of administration, the scheduling of administration, and other factors known to medical practitioners. The “therapeutically effective amount” of a compound to be administered will be governed by such considerations, and can be the minimum amount necessary to prevent, ameliorate, repair, or treat lung disorders. Such amount is preferably below the amount that is toxic to the host or renders the host significantly more susceptible to infections.

The initial pharmaceutically effective amount of the Sox17 or Spdef nucleic acid, or the fragment or analog or derivative thereof compound administered can be in the range of about 0.0001, 0.001, or 0.005, to about 30, 40, or 50 mg/kg of patient body weight per day, being preferably from about 0.01, 0.1, 0.3, 0.5, 1, or 2, to about 4, 8, 10, 12, 15, or 20 mg/kg/day.

As noted above, however, these suggested amounts of compound are subject to therapeutic discretion, including the individual type of compound being used. The key factor in selecting an appropriate dose and scheduling is the result obtained, as indicated above. For example, the compound can be optionally formulated with one or more agents currently used to prevent or treat lung disorders. The effective amount of such other agents depends on the amount of the compound present in the formulation, as well as other factors discussed above. These are generally used in the same dosages and with administration routes as used hereinbefore or about from 1 to 99% of the heretofore employed dosages, with intermediate dosage levels such as those set forth above.

Embodiments of the present invention contemplate formulations comprising the Sox17 or Spdef nucleic acid, or the fragment or analog or derivative thereof, for use in a wide variety of devices that are designed for the delivery of pharmaceutical compositions and therapeutic formulations to the respiratory tract. The preferred route of administration of these embodiments is in the aerosol or inhaled form. The Sox17 nucleic acid, or the fragment or analog or derivative thereof, combined with a dispersing agent, or dispersant, can be administered in an aerosol formulation as a dry powder or in a solution or suspension with a diluent.

As used herein, the term “dispersant” refers to an agent that assists aerosolization of the nucleic acid or absorption of the nucleic acid in lung tissue, or both. Preferably the dispersant is pharmaceutically acceptable. As used herein, the term “pharmaceutically acceptable” means approved by a regulatory agency of the Federal or a state government as listed in the U.S. Pharmacopeia or other generally recognized pharmacopeia for use in animals, and more particularly in humans. Suitable dispersing agents are well known in the art, and include but are not limited to surfactants and the like. For example, surfactants that are generally used in the art to reduce surface induced aggregation of the composition caused by atomization of the solution forming the liquid aerosol can be used. Nonlimiting examples of such surfactants are surfactants such as polyoxyethylene fatty acid esters and alcohols, and polyoxyethylene sorbitan fatty acid esters. Amounts of surfactants used will vary, being generally within the range or 0.001 and 4% by weight of the formulation. In a specific aspect, the surfactant is polyoxyethylene sorbitan monooleate or sorbitan trioleate. Suitable surfactants are well known in the art, and can be selected on the basis of desired properties, depending on the specific formulation, concentration of Sox17 or Spdef nucleic acid, or the fragment or analog or derivative thereof, diluent (in a liquid formulation) or form of powder (in a dry powder formulation), etc.

Moreover, depending on the choice of the Sox17 or Spdef nucleic acid, the desired therapeutic effect, the quality of the lung tissue (e.g., diseased or healthy lungs), and numerous other factors, the liquid or dry formulations can comprise additional components, as discussed further below.

In some embodiments of the invention, liquid aerosol formulations containing the Sox17 or Spdef nucleic acid, or the fragment or analog or derivative thereof, are combined with a dispersing agent in a physiologically acceptable diluent. The dry powder aerosol formulations of the present invention can consist of, for example, a finely divided solid form of the Sox17 nucleic acid, or the fragment or analog or derivative thereof, and a dispersing agent. In general the mass median dynamic diameter can range from less than about 0.5, 1, or 3 μm to more than about 8, 12, 15, 20, or 30 μm. Preferably, the diameter is about 5 micrometers or less in order to ensure that the drug particles reach the lung alveoli (Wearley, L. L., Crit. Rev. in Ther. Drug Carrier Systems, 8:333 (1991), which is incorporated by reference herein in its entirety). The term “aerosol particle” is used herein to describe the liquid or solid particle suitable for pulmonary administration, i.e., that will reach the alveoli. Other considerations such as construction of the delivery device, additional components in the formulation and particle characteristics are important. These aspects of pulmonary administration are well known in the art, and manipulation of formulations, aerosolization means and construction of a delivery device require at most routine experimentation by one of ordinary skill in the art.

Other advantageous carriers include aerodynamically light particles made of a biodegradable material having a tap density of less than about 0.6, 0.8, 1.0, or 1.2 g/cm³. Preferably, the tap density is less than about 0.4 g/cm³. Examples of such particles are presented in Hanes, et al., U.S. Pat. No. 6,136,295, which is incorporated by reference herein in its entirety. Typically the particles are formed of biodegradable polymers, for example, the particles can be formed of a functionalized polyester graft copolymer consisting of a linear alpha hydroxy acid polyester backbone having at least one amino acid group incorporated therein and at least one poly amino acid side chain extending from an amino acid group in the polyester backbone.

With regard to construction of the delivery device, any form of aerosolization known in the art, including but not limited to nebulization, atomization or pump aerosolization of a liquid formulation, and aerosolization of a dry powder formulation, can be used in the practice of the invention. A delivery device that is uniquely designed for administration of solid formulations is envisioned. Often, the aerosolization of a liquid or a dry powder formulation will require a propellant. The propellant can be any propellant generally used in the art. Specific nonlimiting examples of such useful propellants are a chlorofluorocarbon, a hydrofluorocarbon, a hydochlorofluorocarbon, or a hydrocarbon, including trifluoromethane, dichlorodifluoromethane, dichlorotetrafluoroethanol, and 1,1,1,2-tetrafluoroethane, or combinations thereof.

In a preferred aspect of the invention, the device for aerosolization is a metered dose inhaler. A metered dose inhaler provides a specific dosage when administered, rather than a variable dose depending on administration. Such a metered dose inhaler can be used with either a liquid or a dry powder aerosol formulation. Metered dose inhalers are well known in the art.

Once the Sox17 or Spdef nucleic acid, or the fragment or analog or derivative thereof, reaches the lung, a number of formulation-dependent factors can affect the drug absorption. It will be appreciated that in treating an Sox17 or Spdef related disease or disorder, such factors as aerosol particle size, aerosol particle shape, the presence or absence of infection, lung disease or emboli can affect the absorption of the protein. For each of the formulations described herein, certain lubricators, absorption enhancers, stabilizers or suspending agents can be appropriate. The choice of these additional agents will vary depending on the goal. It will be appreciated that in instances where local delivery of the Sox17 or Spdef nucleic acid, or the fragment or analog or derivative thereof; is desired or sought, such variables as absorption enhancement will be less critical.

In a further embodiment, an aerosol formulation of the present invention can include other active ingredients in addition to the Sox17 or Spdef nucleic acid, or the fragment or analog or derivative thereof. In a preferred embodiment, such active ingredients are those used for the treatment of lung disorders. For example, such additional active ingredients include, but are not limited to, bronchodilators, antihistamines, epinephrine, and the like. In another embodiment, the additional active ingredient can be an antibiotic.

In some embodiments of the invention, the Sox17 or Spdef nucleic acid, or the fragment or analog or derivative thereof, is introduced into the subject in the aerosol form in an amount between about 0.01 mg per kg body weight of the mammal up to about 100 mg per kg body weight of said mammal. In preferred embodiments, the Sox17 nucleic acid, or the fragment or analog or derivative thereof; is introduced from about 0.1 mg, 0.5 mg, 1 mg, or 5 mg, to about 25 mg, 50 mg, or 80 mg per kilogram of body weight per day. In a specific embodiment, the dosage is dosage per day. One of ordinary skill in the art can readily determine a volume or weight of aerosol corresponding to this dosage based on the concentration of Sox17 nucleic acid, or the fragment or analog or derivative thereof, in an aerosol formulation of the invention; alternatively, one can prepare an aerosol formulation which with the appropriate dosage of Sox17 nucleic acid, or the fragment or analog or derivative thereof, in the volume to be administered, as is readily appreciated by one of ordinary skill in the art. In some embodiments of the present invention, administration of Sox17 nucleic acid, or the fragment or analog or derivative thereof, directly to the lung allows the use of less nucleic acid, thus limiting both cost and unwanted side effects.

The formulation can be administered in a single dose or in multiple doses depending on the disease indication. It will be appreciated by one of skill in the art the exact amount of prophylactic or therapeutic formulation to be used will depend on the stage and severity of the disease, the physical condition of the subject, and a number of other factors.

Systems of aerosol delivery, such as the pressurized metered dose inhaler and the dry powder inhaler are disclosed in Newman, S. P., Aerosols and the Lung, Clarke, S. W. and Davia, D. editors, pp. 197-22, which is incorporated by reference herein in its entirety, and can be used in connection with the present invention.

In some embodiments of the invention, a liposome formulation can be effective for administration of Sox17 or Spdef nucleic acid, or the fragment or analog or derivative thereof, by inhalation.

The present invention provides aerosol formulations and dosage forms for use in treating subjects suffering from a pulmonary disease or disorder. In general, such dosage forms contain one or more Sox17 or Spdef nucleic acids, or the fragments or analogs or derivatives thereof in a pharmaceutically acceptable diluent. Pharmaceutically acceptable diluents include but are not limited to sterile water, saline, buffered saline, dextrose solution, and the like. In a specific embodiment, a diluent that can be used in the present invention or the pharmaceutical formulation of the present invention is phosphate buffered saline, or a buffered saline solution generally between the pH 7.0-8.0 range, or water.

The liquid aerosol formulation of the present invention can include, as optional ingredients, pharmaceutically acceptable carriers, diluents, solubilizing or emulsifying agents, surfactants and excipients. Such carriers can serve simply as bulking agents when it is desired to reduce' the concentration of the Sox17 or Spdef nucleic acid, or the fragment or analog or derivative thereof, in the powder or liquid which is being delivered to a patient, but can also serve to enhance the stability of the composition and to improve the dispersability of the powder or liquid within a dispersion device in order to provide more efficient and reproducible delivery of the Sox17 or Spdef nucleic acid, or the fragment or analog or derivative thereof, and to improve handling characteristics of the protein or nucleic acid such as flowability and consistency to facilitate manufacturing and powder or liquid filling.

If desired, the formulation can include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half life for clearance. Suitable carrier materials can be in the form of an amorphous powder, a crystalline powder, a combination of amorphous and crystalline powders or a liquid. Suitable materials include carbohydrates, e.g., monosaccharides such as fructose, galactose, glucose, D-mannose, sorbose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; (b) amino acids, such as glycine, arginine, aspartic acid, glutamic acid, cysteine, lysine, and the like; (c) organic salts prepared from organic acids and bases, such as sodium citrate, sodium ascorbate, magnesium gluconate, sodium gluconate, tromethamine hydrochloride, and the like; (d) peptides and proteins, such as aspartame, human serum albumin, gelatin, and the like; (e) alditols, such as mannitol, xylitol, and the like. A preferred group of carriers includes lactose, trehalose, raffinose, maltodextrins, glycine, sodium citrate, tromethamine hydrochloride, human serum albumin, and mannitol,

Such carrier materials can be combined with the Sox17 or Spdef nucleic acid, or the fragment or analog or derivative thereof, prior to administration, i.e., by adding the carrier material to the buffer solution. In that way, the carrier material will be formed simultaneously with and as part of the Sox17 nucleic acid, or the fragment or analog or derivative thereof. Alternatively, the carriers can be separately prepared in a dry powder or liquid form and combined with the Sox17 nucleic acid, or the fragment or analog or derivative thereof, by blending. The size of the carrier particles can be selected to improve the flowability of the powder or liquid.

The liquid or dry aerosol formulations the Sox17 or Spdef nucleic acid, or the fragment or analog or derivative thereof, of the present invention can be aerosolized by dispersion in a flowing air or other physiologically acceptable gas stream in a conventional manner. The liquid aerosol formulations can be used with a nebulizer. The nebulizer can be, for example, compressed air driven, ultrasonic, or the like. Any nebulizer known in the art can be used in conjunction with the present invention such as but not limited to: Ultravent, Mallinckrodt, Inc. (St. Louis, Mo.); the Acorn II nebulizer (Marquest Medical Products, Engelwood Colo.). Other nebulizers useful in conjunction with the present invention are described in U.S. Pat. No. 4,624,251 issued Nov. 25, 1986; U.S. Pat. No. 3,703,173 issued Nov. 21, 1972; U.S. Pat. No. 3,561,444 issued Feb. 9, 1971; and U.S. Pat. No. 4,635,627 issued Jan. 13, 1971, each of which is incorporated by reference herein in its entirety.

The Sox17 nucleic acid or the fragment or analog or derivative thereof formulations of the present invention can also include other agents useful for product stabilization or for the regulation of osmotic pressure. Examples of the agents include but are not limited to salts, such as sodium chloride, or potassium chloride, and carbohydrates, such as glucose, galactose or mannose, and the like.

It is also contemplated that the present pharmaceutical formulation can be used as a dry powder inhaler formulation comprising a finely divided powder form of the Sox17 nucleic acid, or the fragment or analog or derivative thereof, and a dispersant. The form of the composition will generally be a lyophilized powder. Lyophilized forms of Sox17 nucleic acid, or the fragment or analog or derivative thereof, can be obtained through standard techniques.

In another embodiment, the dry powder formulation will comprise a finely divided dry powder containing the Sox17 or Spdef nucleic acid, or the fragment or analog or derivative thereof, a dispersing agent and also a bulking agent. Bulking agents useful in conjunction with the present formulation include such agents as lactose, sorbitol, sucrose, or mannitol, in amounts that facilitate the dispersal of the powder from the device.

The Sox17 nucleic acid, or the fragment or analog or derivative thereof, of the invention is useful in the prophylactic or therapeutic treatment of chemically-induced lung-injury related diseases, biologically-induced lung-injury related diseases, or other lung disorders in which pulmonary administration is desirable or in which the lungs are involved. Likewise, the invention contemplates pulmonary administration of such amounts of the protein that are sufficient either to achieve systemic delivery of a therapeutic or biological amount of the protein, or such amounts that achieve only local delivery of a therapeutic or biological amount of the protein to the lung. The invention further contemplates parenteral administration or pulmonary administration of the Sox17 nucleic acid or protein, as well as fragments or analogs or derivatives thereof.

It will be appreciated by one skilled in the art that approach to systemic or local delivery of the formulation of the Sox17 or Spdef nucleic acid, or the fragment or analog or derivative thereof, will depend on the indication being treated. What constitutes a therapeutically effective amount in a particular case will depend on a variety of factors within the knowledge of the skilled practitioner. Such factors include the physical condition of the subject being treated, the severity of the condition being treated, the disorder or disease being treated, and so forth. In general, any statistically significant attenuation of one or more symptoms associated with a lung injury or a lung disorder constitutes treatment within the scope of the present invention.

It is contemplated that the formulation of the Sox17 nucleic acid, or the fragment or analog or derivative thereof, can be administered to a subject in need of prophylactic or therapeutic treatment. As used herein, the term “subject” refers to an animal, more preferably a mammal, and most preferably a human.

Pulmonary administration of the Sox17 or Spdef nucleic acid, or the fragment or analog or derivative thereof, can be used to result in systemic or local effects. Pulmonary administration of Sox17 or Spdef nucleic acid, or the fragment or analog or derivative thereof, is preferred for the treatment of lung disorders or diseases because of the high local concentration of Sox17 or Spdef that can be delivered.

In some embodiments of the invention, inhibition of Sox17, B-Wnt/catenin, and Spdef can be useful to treat certain diseases, such as, for example, lung cancer. In these situations, inhibiting molecules, such as modified or unmodified nucleic acids, RNAi, and antisense molecules can be administered to the patient using the administration methods described herein. In some embodiments, assays to screen for molecules that inhibit these proteins are also provided.

Assays for Screening Molecules that are Capable of Upregulating Sox17 to Activate Pulmonary Repair

In some embodiments of the invention, screening assays for finding compounds that can upregulate Sox17 are provided. Compounds that are identified in initial screens can be then be further analyzed to confirm their activity. Any suitable compound can be screened. For example, many types of libraries of compounds are available and can be used for screening procedures. Such compounds can include, but are not limited to, natural or synthetic nucleic acids, peptides such as, for example, soluble peptides, combinatorial chemistry-derived molecular libraries, antibodies including, but not limited to, polyclonal, monoclonal, humanized, anti-idiotypic, chimeric or single chain antibodies, and FAb, F(ab′)2 and FAb expression library fragments, and epitope-binding fragments thereof, and small organic or inorganic molecules. Computer modeling and searching technologies can also be used to identify compounds that are suitable for upregulating Sox17. Computer modeling and structural analysis methods, such as X-ray crystallography, can be used to improve on identified compounds. In some embodiments, assays to screen for inhibition of nuclear translocation of Sox17, or assays to screen for the degradation or stabilization of Sox17 or other pulmonary pathway protein, can be used to find new therapeutically useful compositions.

Agents that Upregulate Sox17 Expression

It can be useful to stimulate endogenous Sox17 levels in a patient, by administering agents that upregulate Sox17 expression. Accordingly, further embodiments of the present invention provide methods of screening or identifying proteins, small molecules or other compounds which are capable of inducing the expression of Sox17 genes and proteins. Assays to identify these molecules can be performed, for example, using transformed or non-transformed cells, immortalized cell lines, or can be performed in vivo, for example, utilizing animal model systems. In some embodiments, the assays can detect the presence of increased expression of Sox17 genes or Sox17 proteins on the basis of increased mRNA expression, increased levels of Sox17 protein products, or increased levels of expression of a marker gene. Potential activator molecules include, for example, NFkB, Stat-3 activated Il-6, TGF-α, and EGF. Examples 15 and 16 describe suitable in vitro assay methods.

For example, cells known to express a Sox17 polypeptide, or transformed to express a particular Sox17 polypeptide, can be incubated and one or more test compounds can be added to the medium. The test compound can be, for example, a combinatorial library for screening a plurality of compounds. A variety of other agents can be included in the screening assay. These include agents like salts, neutral proteins, e.g., albumin, detergents, etc that are used to facilitate optimal binding and/or reduce nonspecific or background interactions. Reagents that improve the efficiency of the assay, such as protease inhibitors, nuclease inhibitors, anti-microbial agents, etc., can be used. The mixture of components can be added in any order. Incubations can be performed at any suitable temperature, typically between 4° C. and 40° C. Incubation periods are typically selected for optimum activity, but can also be optimized to facilitate rapid high-throughput screening. After allowing a sufficient period of time, for example, from about 0-72 hours, or longer, for the compound to induce the expression of the Sox17, any change in levels of expression from an established baseline can be detected using any of the techniques known to those of skill in the art.

In some embodiments of the present invention, agents that upregulate expression of Sox17 can be found by screening a library of compounds with a reporter gene construct that is operably linked to a Sox17 promoter. A demonstration of this type of assay is discussed in Example 15. A compound can affect reporter gene expression by either stimulating or inhibiting the expression of the reporter gene. Thus, an agent that is capable of upregulating Sox17 will be able to turn on the reporter gene. A compound “stimulates” reporter gene expression if the level of transcripts or protein product produced from the reporter gene is increased. One of skill in the art can identify a number of reporter genes for use in the screening method of the invention. Examples of reporter genes for use with the invention include but are not limited to beta-galactosidase, green fluorescent protein, alkaline phosphatase, luciferase, and the like. These reporter genes are preferably operably joined to a Sox17 5′ regulatory region in a recombinant construct.

The effect of the compound on the reporter gene transcription can be measured by assessing the expression of the reporter by methods well known in the art (e.g., Northern blots; EMSA). Alternatively or the production of protein product from the reporter gene can be measured by methods well known in the art (e.g., ELISA or RIA; Western blots; SDS-PAGE).

Candidate agents can be obtained from a wide variety of sources including libraries of synthetic or natural compounds. One class is organic molecules, preferably small organic compounds having a molecular weight of more than 50 and less than about 2,500 daltons. Numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and can be used to produce combinatorial libraries. Known pharmacological agents can be subjected to directed or random chemical modifications, such as acylation, alkylation, esterification, amidification, etc., to produce structural analogs. Candidate agents are also found among biomolecules including, but not limited to: peptides, saccharides, fatty acids, steroids, purines, pyrimidines, derivatives, structural analogs or combinations thereof.

Candidate agents that are found to increase Sox17 mRNA or protein levels in the above assays can be tested further in animal models. For example, a mouse model of pulmonary damage can be initiated by administering naphthalene to mice as described in Example 1. Candidate agents can then be administered to the mouse lungs, and the levels of Sox17 mRNA or protein can be measured. Additionally, tissue samples can be taken to study the repair process. Further, expression of β-catenin and Stat-3 can be determined. The measurements can be compared with that of control naphthalene-treated mice to determine which agents speed the repair process. Example 18 demonstrates the use of a mouse model of pulmonary repair to confirm the ability of the candidate agents to upregulate Sox17.

The most promising candidate agents can be tested further for the possibility of use as human pharmaceutical compositions for speeding lung repair after injury. Example 19 demonstrates the administration of a Sox17 upregulating agent to treat pulmonary damage in a human patient.

Additional embodiments of the present invention provide methods of identifying proteins, small molecules and other compounds on the basis of their ability to modulate the activity of Sox17, the activity of other Sox17-regulated genes, the activity of proteins that interact with Sox17 proteins, the intracellular localization of Sox17, changes in transcription activity, the presence or levels of Sox17, or other biochemical, histological, or physiological markers which distinguish cells bearing normal and modulated Sox17 activity.

### Antibodies to Sox17 or Spdef as Diagnostic Agents and Diagnostic Kits

In some embodiments of the invention, antibodies to Sox17, Spdef, β-catenin, and/or Stat-3 can be used as tools to determine the status of pulmonary tissue. For example, the antibodies can be used to determine whether lung repair after injury is occurring, or to help determine the degree of damage a lung has sustained. The antibodies can also be used as part of a diagnostic kit, if desired.

Antibodies having specific binding affinity to a polypeptide can be used in methods for detecting the presence and/or amount of a polypeptide in a sample by contacting the sample with the antibody under conditions such that an immunocomplex forms and detecting the presence and/or amount of the antibody conjugated to the polypeptide. By “specific binding affinity” is preferably meant that the antibody binds to the target polypeptides with greater affinity than it binds to other polypeptides under specified conditions. In some embodiments, diagnostic kits for performing such methods can be constructed to include a first container containing the antibody and a second container having a conjugate of a binding partner of the antibody and a label, such as, for example, a radioisotope. The diagnostic kit can also optionally include notification of an FDA approved use and instructions therefor.

One skilled in the art will appreciate that these methods and devices can be adapted to carry out the objects of the invention, and obtain the ends and advantages mentioned, as well as those following therefrom. The methods, procedures, and devices described herein are presently representative of preferred embodiments and are exemplary and are not intended as limitations on the scope of the invention. Changes therein and other uses can occur to those skilled in the art which are encompassed within the spirit of the invention and are defined by the scope of the disclosure.

It will be apparent to one skilled in the art that varying substitutions and modifications can be made to the invention disclosed herein without departing from the scope and spirit of the invention.

### EXAMPLES

The following examples are offered to illustrate, but not to limit, certain embodiments of the invention.

### Example 1

### Preparation of Transgenic Mice and Naphthalene Treatment

To determine whether Sox17 can play a role in regenerating pulmonary epithelium following pulmonary injury, Sox17 expression in mice was measured after the bronchioles were denuded by intraperitoneal injection of naphthalene (Van Winkle et al., Am. J. Physiol., 269:L800-L818 (1995); Stripp, 1995 Am J Physiol. 1995 December; 269 (6 Pt 1):L791-9; each of which is incorporated by reference herein in its entirety). Female FVBIN mice (12 weeks old) were obtained from Charles-River and housed under pathogen-free conditions. Naphthalene (Sigma Chemical Co., St Louis, Mo.) was dissolved in corn oil at a concentration of 30 mg/ml and administered to mice (275 mg/kg) via intraperitoneal injection (Reynolds et al., Am. J. Pathol., 156:269-278 (2000); Hong et al., Am. J. Respir. Cell Mol. Biol., 24:671-681 (2001), each of which is incorporated by reference herein in its entirety). Control mice received corn oil. Animals were anesthetized with halothane before injection. ((tetO)7-CMV-Sox17 or (tetO)7-CMV-tSox17 transgenic mice were produced by oocyte injection of a plasmid construct consisting of cDNAs of full-length Sox17, IRES (Internal Ribosome Entry Site) sequence, and cDNA encoding nuclear enhanced green fluorescent protein (NLS-EGFP) with nuclear localization signal peptide (NLS) fused to the NHz-terminus of enhanced green fluorescent protein (EGFP). The (tetO)7 region contained seven copies of tetO, the tet repressor protein (tetR) binding site. tSox17 is a truncated (t) mouse Sox17 cDNA that lacks the HMG box domain (Kanai et al., J. Cell. Biol., 133:667-681 (1996), which is incorporated by reference herein in its entirety). Both Sox17 and tSox17 were expressed in respiratory epithelial cells under conditional control of doxycycline using transgenic mice as described in (Perl et al., Transgenic Res., 11:21-29 (2002), which is incorporated by reference herein in its entirety). hSP-C-rtTA and rCCSP-rtTA mice were mated to (tetO)7-CMV-Sox17 or tSox17 bitransgenic mice. To induce Sox17 or tSox17 expression, bitransgenic mice were maintained on doxycycline-containing food (25 mg/g; Harlan Teklad, Madison, Wis.) from E6.5 (hSP-C-rtTA/Sox17) or from 2 months of age (rCCSP-rtTA/Sox17 or tSox17) until the time of sacrifice as described in specific experiments. Single transgenic littermates of all other genotypes served as controls. hSP-C-rtTA/(tetO)7-Cre/ZEG triple transgenic mice were generated as described in (Perl et al., Proc. Natl. Acad. Sci. USA, 99:10482-10487 (2002), which is incorporated by reference herein in its entirety). When these dams were treated with doxycycline, recombination permanently induced expression of green fluorescent protein in respiratory epithelial cells in the fetal lung. Dams bearing triple transgenic pups were treated with doxycycline as described above from E6.5 until birth. The triple transgenic mice were maintained on doxycycline until administration of naphthalene and sacrifice for analysis. The mice were housed and maintained in pathogen-free conditions according to protocols approved by the Institutional Animal Care and Use Committee at Cincinnati Children's Hospital Research Foundation. Mice were anesthetized with a mixture of ketamine, acepromazine, and xylazine, and exsanguinated by severing the inferior vena cava and descending aorta. Pneumonectomy was performed in adult mice as described by (Leuwerke et al., Am. J Physiol., 282:L1272-L1278 (2002), which is incorporated by reference herein in its entirety). Sham operated and ungenerated controls and post-pneumonectomy mice were killed (n=4 per group) for analysis.

### Example 2

### Immunohistochemistry Methods

To examine the spatial and temporal expression of Sox17, β-catenin, and Stat-3 in pulmonary tissues, the following method was used. Lungs of embryonic and adult mice were fixed in 4% paraformaldehyde/PBS for 15 to 24 hours at 4° C. and processed according to standard methods for paraffin-embedded blocks. Immunohistochemistry was performed on 5 μm thick sections using antibodies against FoxJ1, CCSP, β-tubulinIV, and β-catenin as previously described (Mucenski et al., J. Biol. Chem., 278:40231-40238 (2003); Wert et al., Dev. Biol., 242:75-87 (2002); Wan et al., Development, 131:953-964 (2004); Zhou et al., Dev. Biol., 175:227-238 (1996), each of which is incorporated by reference herein in its entirety). Guinea pig anti-Sox17 antibody was raised against a synthetic peptide composed of amino acids 249-410 of mouse Sox17. A distinct rabbit anti-Sox17 antibody was generated against mouse Sox17 (Sinner et al., Development, 131:3069-3080 (2004), which is incorporated by reference herein in its entirety). Antibodies against cytokeratin-5 and phosphohistone-3 were used at dilution of 1:2000 and 1:500, respectively (Research & Diagnostics Inc., NJ and US Biological, MA). For dual immuno-labeling procedures, antibodies from two different species were used as follows: antiSox17, 1:100; antiCCSP, 1:500; antiproSP-C, 1:100; anti β-tubulinIV, 1:50; antiFoxa2, 1:100; and phosphohistone-3, 1:50. Goat or donkey secondary antibodies were conjugated with Alexa Fluor@ 568 or Alexa Fluor@ 488 fluorchrome (Molecular Probes, Eugene, Oreg.). The samples were mounted with anti-fade reagent containing the fluorescent nucleic acid stain 4′,6-diamidino-2-phenylindole DAPI (Vecta Shields, Burlingame, Calif.).

### Example 3

### Transmission Electron Microscopy Methods

To prepare tissue samples for electron microscopy, adult mouse lungs were inflation-fixed via a tracheal cannula at 25 cm of water pressure using modified Karnovsky's fixative (2% glutaraldehyde and 2% paraformaldehyde in 0.1 M sodium cacodylate buffer (SCB) containing 0.1% calcium chloride (pH 7.3)). The tissue was post-fixed with 1% osmium tetroxide (reduced with 1.5% potassium ferrocyanide). The tissue was then stained en bloc with aqueous 4% uranyl acetate and processed for electron microscopy.

### Example 4

### Vector Construction, Cell Culture and Transfection Assay

The Sox17 expression vector was generated by amplifying Sox17 from E7.5 endoderm cDNA and inserting into the XhoI sites of the pCIG vector (Megason et al., Development, 129:2087-2098 (2002), which is incorporated by reference herein in its entirety). The reporter gene construct 1.1 mFoxj1-pGL3 was generated by amplifying 1.1 kb region (−1044 to +79) of Foxj1 5′ flanking region using tail genomic DNA from FVBIN mouse and cloning into pGL3 basic luciferase plasmid (Promega, Madison, Wis.). PCR primers were designed based on the nucleotide sequence at GenBank Accession No. AF006200 (SEQ ID NO: 11). The sequence of the primers was: forward 5′-GGT ACC AAA GAC TTC AAG GGC ACG-3′ (SEQ ID NO: 12) and reverse 5′-AGA TCT GCC AGT TAC ACA GTC TCC AG-3′ (SEQ ID NO: 13).

HeLa cells were plated at 1×10⁵ cells/well in 12-well plates. The reporter construct mFoxj1-pGL3 was co-transfected with empty vector or pCIG-Sox17 and pCMV-β-galactosidase using Lipofectamine-2000 according to the manufacturer's protocol (InVitrogen, Carlsbad, Calif.). After 48 hours of incubation, lysates were assayed for β-galactosidase and luciferase activities (Promega, Madison, Wis.). Light units were assayed by luminometry (Monolight 3010, Analytical Luminescence Laboratory, San Diego, Calif.) and normalized to β-galactosidase activity.

### Example 5

### Ciliated Epithelial Cells are a Source of Progenitor Cells During Repair of Airway Epithelium

In order to determine whether ciliated epithelial cells can act as progenitor cells after airway injury, the following experiment was performed. Mice were injected with naphthalene as described in Example 1 to denude the bronchioles. The bronchiolar surface was examined microscopically twenty-four hours after injection. The results are shown in FIG. 1.

FIG. 1(a) shows hematoxylin-eosin staining of mouse bronchioles 24 hours after administration of naphthalene demonstrates exfoliation of the epithelium. While the bronchiolar surface appeared to be denuded at the light microscopic level (FIG. 1a), the conducting airways were actually lined by a homogenous population of thin squamous cells. The endodermal origin of these cells was identified using hSP-C-rtTA/(tetO)7-Cre/ZEG mice.

In FIG. 1(b), green fluorescent protein (GFP) was observed in the squamous progenitor cells lining the conducting airway in hSP-C-rtTA/(tetO)7CMVCre/ZEG mouse 24 hours after naphthalene treatment. The GFP-positive cells in peripheral lung parenchyma (white arrow) are alveolar type II cells.

In these transgenic mice, GFP is expressed after conditional excision-activation of a foxed ZEG gene during fetal lung development (Pal et al., Proc. Natl. Acad. Sci. USA, 99:10482-10487 (2002), which is incorporated by reference herein in its entirety). All of the squamous cells lining the “denuded” bronchioles were fluorescent, indicating their origin from a subset of endodermally-derived bronchiolar epithelial cells (FIG. 1b). Squamous metaplasia of the injured cells occurred within 6-12 hours and preceded sloughing of the nonciliated cells.

The presence of squamous cells lining the airways was then confirmed using electron microscopy (FIG. 1c). Electron micrographs were prepared 24 hours after naphthalene treatment, showing squamous cells with few cilia (black arrowhead). A necrotic, nonciliated cell were present in the airway lumen (asterisk). Disorganized cilia (and components of cilia) were present on and within the squamous cells, as shown in FIGS. 1c and 1d. Basal bodies (white arrowhead) and internalized cilia were observed with the squamous cells that homogeneously lined all of the bronchioles (FIG. 1d).

Microscopic immunofluorescence imaging (FIG. 2) demonstrated that ciliated cells can serve as progenitor cells during repair of airway epithelium. Double immunolabeling for clara cell secretory protein (CCSP) (red) and β-tubulin (green) (a-e), and Foxj1 staining (f-j) was performed on lung sections of uninjured control (a, f) and naphthalene treated mice 1-14 days after injection. One day after injury, squamous cells lining injured airways stained for β-tubulin and Foxj1, but not CCSP (b), while exfoliated cells in the lumen stained for CCSP. Two days after injury, Foxj1 was detected in nuclei of cuboidal cells that now lined the bronchioles wherein β-tubulin staining was primarily intracellular and disorganized. CCSP staining was not detected (c, h). Four days after injury, a subset of epithelial cells was weakly stained for CCSP (d). Fourteen days after injury, the normal staining pattern of CCSP and β-tubulin was restored (e). Foxj1 was restricted to a subset of cells 4 and 14 days after injury (i, j).

Clara cells, identified by immunostaining with anti-CCSP antibody, were selectively shed into the airway lumen and the remaining squamous cells expressed both β-tubulin and Foxj1 indicating their origin from ciliated cells. These squamous cells did not express CCSP 24 hours after injury (FIG. 2b). Phosphohistone-3 stained epithelial cells were not observed at 24 hours, but were detected 2-4 days after injury, indicating that epithelial integrity was initially maintained by extension and migration of the squamous cells in a process that did not require proliferation. Within 48 hours after injury, the squamous cells lining the injured bronchioles had transformed to a homogenous population of cuboidal cells. β-tubulin and Foxj1 (ciliated cell markers), but not CCSP staining, was detected in these transitional cuboidal cells (FIGS. 2b, g). These cuboidal cells represent an intermediate or amplifying pool of cells, derived from ciliated cells that labeled with phosphohistone-3 indicating mitotic activity. BrdU labeling experiments were consistent with those of phosphohistone-3. In contrast to its apical localization in normal lung, β-tubulin staining was localized throughout the intracellular compartment and organized cilia were not seen in the squamous and cuboidal cells (FIGS. 1c, d). Clara cell morphology was not observed until 4-7 days after injury, at which time CCSP was detected, albeit at low levels, in subsets of airway cells (FIG. 2d). Fourteen days after injury, morphology of the bronchiolar epithelium and the distinct pattern of staining of CCSP (in Clara cells), as well as Foxj1 and β-tubulin (in ciliated cells) was substantially restored (FIGS. 2e, j). These findings demonstrate that following naphthalene injury, ciliated cells undergo squamous metaplasia and redifferentiate into both ciliated and non-ciliated cell types.

### Example 6

### Epithelial Cell Injury Results in Transcriptional Reprogramming

To examine transcriptional changes during pulmonary repair, the expression of Foxa1, Foxa2, Foxj1, and TTF-1 was determined using immunohistochemical methods as described in Example 2. The results are shown in FIG. 3.

Dynamic changes in expression of Foxa1, Foxa2, and TTF-1 occurred during repair. Prior to injury, Foxa2 and Foxa1 were detected primarily in a subset of bronchiolar epithelial cells (black arrow), while TTF-1 was expressed more widely (d 0). Dual labeling for Foxa2 (red) and β-tubulin (green) demonstrated that Foxa2 was most intense in β-tubulin-positive ciliated cells (day 0, inset, white arrow). Twenty four to 48 hours after injury, all squamous and cuboidal cells were positive for Foxa2 and Foxa1 (day 1, 2). Four days after injury, Foxa2 and Foxa1 were again restricted to ciliated cells.

The expression pattern of p-catenin was also examined (FIG. 4). FIG. 4 is a microscopic image demonstrating Sox17 and β-catenin staining in progenitor cells during repair of airway epithelium. Prior to injury (d 0), Sox17 (red) was detected in β-tubulin (green) positive ciliated cells (inset f). Sox17 and β-catenin staining were observed in squamous cells 1 day after injury (b, g) and cuboidal cells 2 days after injury (c, h). β-catenin staining is normally cytosolic and membrane-associated and rarely observed in nuclei of airway epithelial cells (FIG. 4a). Twenty-four to 48 hours after pulmonary injury, nuclear and cytoplasmic staining for β-catenin was markedly increased in the squamous and cuboidal cells lining the bronchioles (FIGS. 4b, c). Four days after injury and afterward, β-catenin staining decreased and was restored to the pattern seen in the normal adult lung (FIGS. 4d, e). On day 4 and 14, Sox17 staining became restricted to subsets of cells (d, e). These findings demonstrate that a dynamic transcriptional program, similar to that observed during normal lung morphogenesis, coordinates the squamous metaplasia and re-differentiation of the progenitor cells following naphthalene injury.

### Example 7

### Expression of Sox17 in the Progenitor Cells of Airway Epithelium Following Injury

To examine the expression of Sox17 after airway injury, Adult mice were given a naphthalene treatment as described in Example 1 to cause injury to the airway epithelium. The expression of Sox17 was then examined periodically during the repair process, using immunohistochemical methods as described in Example 2. Sox17 was selectively expressed in ciliated respiratory epithelial cells, and co-localized with β-tubulin and Foxj1 (FIG. 4a). Intense Sox17 staining was observed in all of the squamous and cuboidal cells lining the bronchioles 24-48 hours after injury (FIGS. 4g, h). Four days after injury and thereafter, Sox17 staining was again increasingly restricted to ciliated cells, a pattern similar to that of β-tubulin, Foxa1, Foxa2, and Foxj1 (FIGS. 4i, j). These findings indicate that Sox17 can regulate expression of genes in ciliated cells or the progenitor cells derived from them, during regeneration of the bronchiolar epithelium, just as it does in early endoderm formation.

### Example 8

### Induction of Sox17, β-catenin, Foxa2, And Foxj1 During Lung Regeneration Following Unilateral Pneumonectomy

To determine whether surgery damage results in a similar induction of transcriptional programs as is induced by napthalene-induced damage, a pneumonectomy procedure was performed, as shown in FIG. 5.

FIG. 5 demonstrates the expression of Sox17, β-catenin, Foxa2, Foxj1, and CCSP/β-tubulin during compensatory growth following pneumonectomy. Immunohistochemistry was performed on the right lung 3 days (a, c, e) and 7 days (b, d, f, g, h) after left pneumonectomy. Numbers of cells that stained for Sox17, β-catenin, Foxa2, and Foxj1, were increased in the bronchiolar epithelium 7 days after pneumonectomy (b, d, f, g), and were similar to sham controls on day 3. Phosphohistone-3 immunostaining (red in g inset) was detected in β-tubulin(green) positive ciliated cells (arrow, g inset).

Hyperplasia of both peripheral (alveolar) and bronchiolar epithelia was observed following pneumonectomy. The extent and intensity of Sox17 and Foxj1 staining were increased following pneumonectomy (FIGS. 5b, g). Likewise, β-catenin and Foxa2 staining was enhanced in ciliated (β-tubulin stained) cells (FIGS. 5d, f). In this model, bronchiolar hyperplasia was associated with increased numbers of both ciliated and Clara cells (FIG. 5h). Phosphohistone-3 staining was readily detected on day 7, but not on day 3 post-surgery, and was observed in multiple cell types, including ciliated and Clara cells in the bronchioles (FIG. 5g inset) and type II cells in the alveoli (latter not shown). Thus, ciliated cells regained proliferative capacity and also used similar transcriptional pathways as in repair that occurs after naphthalene treatment.

### Example 9

### Sox17 Increased Nuclear B-catenin and Altered Epithelial Differentiation In vivo

Sox17 was expressed in respiratory epithelium of fetal mice under conditional control of the SP-C promoter (Perl et al., Transgenic Res., 11:21-29 (2002), which is incorporated by reference herein in its entirety).

Microscopy was used to demonstrate that Sox17 can induce Foxj1 and β-catenin in vivo, as shown in FIG. 6. Expression of Foxj1, β-catenin, and Sox17 was assessed in lungs from transgenic fetal mice expressing Sox17 (hSP-C-rtTA/(tetO)7Sox17) at E18.0, Sox17 disrupted branching morphogenesis and altered differentiation of epithelial cells lining the lung tubules at E 18, producing a hyperplastic bronchiolar epithelium (FIGS. 6b-f). Expression of Sox17 was accompanied by increased nuclear β-catenin staining and widespread, ectopic expression of Foxj1 and β-tubulin (FIGS. 6b, d). Most of the epithelial cells lining the lung tubules of the transgenic mice expressing Sox17 contained apical cilia. Thus, Sox17 alone was sufficient to induce a ciliated cell phenotype during fetal lung morphogenesis.

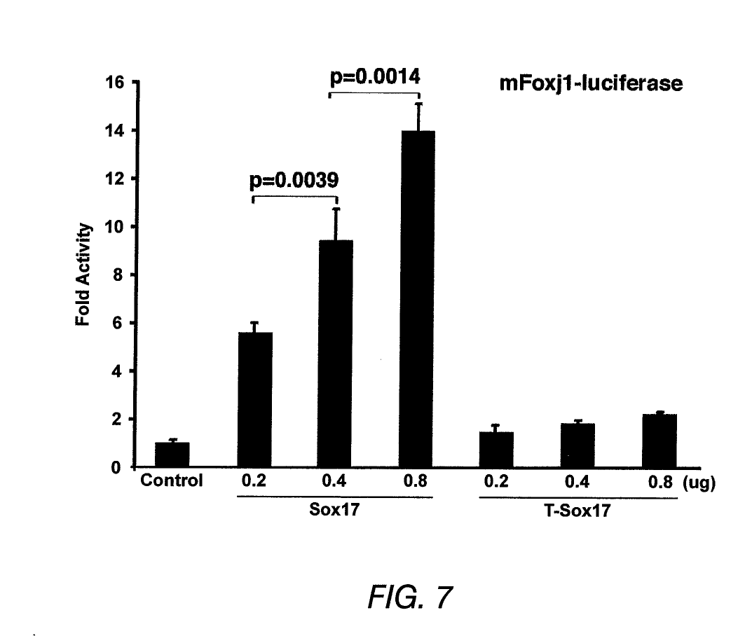

Because Sox17 was co-expressed with Foxj1 and induced production of ciliated cells in the fetal lung in vivo, it was then tested whether Sox17 regulated the expression of the Foxj1 promoter in vivo. Foxj1 is required for ciliogenesis in the lung and other organs (Chen et al., J. Clin. Invest., 102:1077-1082 (1998); Brody et al., Am. J. Respir. Cell. Mol. Biol., 23:45-51 (2000), each of which is incorporated by reference herein in its entirety). The results are shown in FIG. 7. Transfection of HeLa cells with a Sox17 expression plasmid increased Foxj1 promoter activity by approximately 5-6 fold.

### Example 10

### Sox17 Induced Progenitor Cell Behavior In vivo

To determine whether Sox17 can influence progenitor cell behavior in vivo, conditional expression of Sox17 in respiratory epithelial cells was performed, as shown in FIG. 8. rCCSP-rtTA/(tetO)7Sox17 transgenic mice were treated with doxycycline (dox) for 8 weeks from 2 months of age. Immunostaining was performed on lung sections of transgenic mice untreated (FIG. 8a-c) or treated (FIG. 8d-o) with dox.

The conditional expression of Sox17 in respiratory epithelial cells in adult mice under control of the CCSP promoter caused formation of atypical, multicellular, epithelial cell clusters at sites of Sox17 production in the peripheral lung (FIG. 8d). In these mice, Sox17 was induced primarily in the alveolar epithelium of adult lung (Perl et al., Transgenic Res., 11:21-29 (2002), which is incorporated by reference herein in its entirety).

CCSP (FIG. 8e), Foxj1 (FIG. 8f), cytokeratin-5 (FIG. 8h), and mucin (FIG. 8i), normally conducting airway specific markers, were detected in alveolar epithelial cells after expression of Sox17 and were not expressed in the absence of doxycycline (b, c and not shown). β-catenin staining was increased in the atypical cell cluster (arrow) induced by Sox17 (FIG. 8g). Sox17 (FIG. 8j, k, l) did not co-localize with proSP-C (FIG. 8j) or CCSP (FIG. 8k), but was detected at varying levels in cells expressing β-tubulin (FIG. 8l). Distinct and overlapping subsets of cells expressed CCSP (FIG. 8m, n), β-tubulin (FIG. 8l, m, o, arrowhead) or proSP-C (FIG. 8n, o), demonstrating that the ectopic expression of Sox17 in the alveolar regions was sufficient to induce hyperplastic lesions and distinct airway epithelial cell types.

The hyperplastic lesions consisted of cuboidal and columnar epithelial cells, some of which stained intensely for Sox17 (FIG. 8d) and β-catenin (FIG. 8g). Distinct cells within the clusters expressed CCSP, Foxj1, β-tubulin, cytokeratin-5 (Ck5) or mucin (MUC5A/C), markers that are normally restricted to conducting airways, but are not normally expressed in alveolar regions of the normal lung (FIGS. 8a-c). The cells expressing conducting airway epithelial markers did not express proSP-C, a specific marker of peripheral respiratory epithelial cells (FIGS. 8n, o). Cells within the abnormal clusters that stained for β-tubulin, a specific marker for ciliated cells, were distinct from those stained with CCSP, a Clara cell marker, FIG. 8m. The intensity of Sox17 staining varied in a consistent manner in the hyperplastic cell clusters. In general, MUC5A/C (a goblet cell marker) and cytokeratin-5 (an upper airway marker) were associated with high levels of Sox17. In contrast, Sox17 staining was less intense in those cells expressing β-tubulin (FIG. 8l), while Sox17 was not detected in CCSP (FIG. 8k) or proSP-C staining cells (FIG. 8j). Co-expression of Sox17 with β-tubulin, Foxj1, and other airway markers is consistent with the observation that Sox17 is present in conducting airway but not alveolar cells in the normal adult mouse lung. In the absence of doxycycline, the Sox17 transgene was not induced in the peripheral lung and ectopic staining of CCSP and Foxj1 was not detected (FIGS. 8a-o).

### Example 11

### Preparation and Purification of a Vector Encoding Sox17 Protein for Pulmonary Administration

A plasmid vector suitable for pulmonary administration to humans is altered to add a nucleic acid encoding Sox17, along with a suitable promoter. The vector material is prepared on a large-scale basis and is purified and tested for the ability to express Sox17 when administered to a human pulmonary system. The Sox17 vector is then mixed with a suitable agent for aerosol administration.

### Example 12

### Treatment of a Pulmonary Injury in Humans by Administration of a Plasmid Vector Encoding Sox17 with a Metered Dose Inhaler

A patient diagnosed with a pulmonary injury is treated with a Sox17-encoding nucleic acid. The patient self-administers a 5 mg/kg dose of a composition containing the Sox17-encoding nucleic acid twice a day, once in the morning and once in the evening, administered using a metered dose inhaler. Improvement of pulmonary function in the patient is monitored by weekly pulmonary X-ray analysis. The pulmonary damage is repaired through this treatment regimen.

### Example 13

Treatment of a Pulmonary Injury in Humans by Administration of a Plasmid Vector Encoding Spdef with a Metered Dose Inhaler

A patient diagnosed with a pulmonary injury is treated with an adenoviral vector having an SPDEF-encoding nucleic acid. The vector composition is administered intratracheally at 1×10⁹ pfu per dose, three times per day, using a metered dose inhaler. To determine the effectiveness of the treatment, cell samples are taken daily, by bronchial brushing to obtain cells. The pulmonary damage is repaired through this treatment regimen.

### Example 14

### Treatment of Pulmonary Damage Due to Excess Smoke Inhalation by Hospital Administration of Sox17-Encoding Nucleic Acid

A hospitalized patient diagnosed with severe pulmonary smoke inhalation damage is treated once every 2 hours with an inhaled formulation containing 0.25 mg of Sox17-encoding nucleic acid vector per kg body weight, using a nebulizer. Improvement of the pulmonary tissues is monitored. The pulmonary damage is ameliorated or repaired through this treatment regimen.

### Example 15

### Treatment of a Bacterially-Induced Pulmonary Disease that Causes Damage to the Pulmonary Tissues by Treating with Sox17-Encoding Nucleic Acid in Combination with an Antibiotic Agent

A patient diagnosed with pulmonary damage caused by a bacterial organism is treated with a combination of the antibiotic amoxicillin at 4 times per day, plus inhalation of a pharmaceutical formulation of Sox17-encoding nucleic acid, at 2 mg/kg/day, administered twice a day. The bacterial infection is reduced, and the lung damage is ameliorated using this combination treatment regimen.

### Example 16

### In vitro Assay to Determine Agents that Upregulate a Sox17 Reporter Construct

In order to find activators or inhibitors of the Sox17 pathway, a plasmid vector is prepared having the Sox17 promoter fused to the EGFP-encoding gene. The vector is transformed to a mammalian host cell line. An array of 1,000 candidate chemical compounds is prepared. The compounds are contacted with the cells containing the Sox17 promoter-EGFP nucleic acid. After 6 hours, the production of the reporter protein is measured. Possible positive candidates are determined and used for further testing.

### Example 17

### In vitro Assay to Determine Agents that Upregulate Sox17

Positive candidates from the above assay are chosen for further testing. An appropriate human cell line is cultured. The candidate compounds are added to the culture. At 2 hours, 4 hours, 6 hours, and 12 hours, expression of Sox17 mRNA is measured. Additionally, levels of Sox17 protein are measured, and compared to cells not having added agents. Candidate compounds that are capable of inducing Sox17 are chosen for further analysis.

### Example 18

### Effect of Sox17 Administration in a Mouse Repair Model

Mice are treated with naphthalene following Example 1, in order to cause damage to the pulmonary epithelium. Immediately following the napthalene treatment, mice are treated with an intratracheal administration of an adenoviral vector encoding Sox17. Healing of the epithelial layer is compared to that of control naphthalene-treated mice that did not receive Sox17 administration. By the use of this method, the mice treated with the Sox17-encoding vector are able to heal more quickly than control mice not receiving a nucleic acid.

### Example 19

### Testing of Candidate Sox17 Upregulating Agents in a Mouse Model of Pulmonary Repair

Mice are treated with naphthalene as described in Example 1. The mice are then treated with candidate agents chosen from Example 16. 12 hours after pulmonary administration of the candidate agents, Sox17 mRNA and protein levels are measured. Further, microscopy is used to determine the status of the naphthalene-damaged tissues. By this method, candidate agents for use in human pharmaceuticals are determined.

### Example 20

### Treatment of Pulmonary Injury by Administration of Agents that Upregulate Sox17 Expression in Pulmonary Tissue

A patient diagnosed with a pulmonary injury is treated with an agent that upregulates Sox17 expression. The patient self-administers a 1 mg/kg dose of the agent, twice a day, using a metered dose inhaler. Results are measured bi-weekly. The pulmonary injury is repaired through this treatment regimen.

The following examples relate to the Spdef protein, which has been found to activate transcription of Sox17.

### Example 21

### Spdef Plasmids, PCR methods, and Antibodies

Generation of Plasmids and Antibodies: cDNA was prepared by reverse-transcription using total RNA isolated from cultured cells of trachea and lung parenchyma. Spdef cDNA containing the entire open reading frame was amplified from fetal lung (E18) using PCR primers (forward 5′-CTT CTG ACA GCA GGC GGC TAA C-3′ (SEQ ID NO: 14); reverse 5′-GAC TGG ATG CAC AAA TTG GTA GAC AAG-3′ (SEQ ID NO: 15);) based on the sequence of the GenBank accession number NM013891 (25). The amplified PCR product was cloned into pCDNA 3.1/V5-His-Topo (InVitrogen, Carlsbad, Calif.). The GST-Spdef cloned insert was sequenced and confirmed with the data base sequence. TTF-1 and the TTF-1 deletion plasmid constructs including: Δ3 TTF-1 (NH₂-terminal deletion) and Δ14 TTF-1 (COOH-terminal deletion), were previously described and kindly provided by Dr. M. deFelice (deFelice, et al. J. Biol. Chem. 278:35574-35583; Guazzi, et al. 1990. EMBO J. 9:3631-3639, each of which is incorporated by reference herein in its entirety). TTF-1 plasmids used for mammalian two-hybrid assays were previously described as summarized in Table 1 (Liu, et al. J. Biol. Chem. 277:4519-4525, which is incorporated by reference herein in its entirety). The reporter plasmids, Sftpa-luc (1.1 kb mouse Sftpa promoter-pGL3), Scgb1a1-luc (2.3 kb rat Scgb1a1 promoter-pGL2), Sftpc-luc (4.8 kb mouse Sftpc promoter-pGL2), MUC5A/C-luc (4.0 kb human MUC5A/C promoter-pGL2), Foxj1-luc (1.1 kb mouse Foxj1 promoter-pGL3), and pG5-luc express the luciferase gene under control of each promoter (Park, et al. J. Biol. Chem. 279:17384-17390; Besnard, et al. 2005. Am. J. Physiol. 289:L750-L759; Li, et al. 1998. J. Biol. Chem. 273:6812-6820; Park, et al. 2006. Dev. Biol. 294:192-202, each of which is incorporated by reference herein in its entirety). For 0.62 and 0.25 Foxj1-luc plasmids, 1.1 kb Foxj1-luc was cut at Xmn1 and Msc1 sites and ligated, respectively. For m1 and m2 mutations, 143 base pair oligonucleotides containing each mutation were introduced by utilizing the unique Bsu36I restriction site (3′ end position −231 to −222 from ATG) within the 0.25 Foxj1-luc and Nhe1 site. The wild-type sequence was removed by digesting 0.25 Foxj1-luc plasmid with Nhe1 and Bsu36I and replaced with the oligonucleotides that were annealed and then digested with Nhe1 and Bsu36I. Sox17-luc was constructed by cloning 3.6 kb 5′ untranslated region (UTR), of mouse Sox17 gene in pGL3B (Promega, Madison, Wis.). The Sox17 UTR was amplified from the tail genomic DNA of adult FVB/N mice using Expand High Fidelity kit (Roche, Indianapolis, Ind.) and the following primers: forward 5′-TTG ACG CGT GTT ATC TTA GAG TCC GCC G-3′ (SEQ ID NO: 16), reverse 5′-AAA CTC GAG ATG GCT CTC CAG ACC GAC-3′ (SEQ ID NO: 17). The PCR fragments were cloned into pGL3 Basic using MluI and XhoI sites (underlined) and sequenced. Guinea pig polyclonal antibodies were generated against a fragment of recombinant Spdef protein (a.a. 3 to 243) fused to a 6× His-tag. A partial cDNA encoding amino acid 3 to 243 of Spdef was amplified and cloned into an E. coli expression vector pTrcHis-TOPO vector (InVitrogen, Carlsbad, Calif.). The recombinant Spdef peptide was expressed in E. coli and purified using nickel chromatography affinity according to the manufacturer's instructions (Novagen, Madison, Wis.).

TABLE 1
List of Constructs

| | |
|---|---|
| pACT-Spdef | Spdef-(1-326) |
| pBIND-TTF-1 | TTF-1-(1-372) |
| pBIND-T1-HD | TTF-1-(161-223) |
| pBIND-T1-C | TTF-1-(224-372) |
| pBIND-T1ΔN | TTF-1-(161-372) |
| pBIND-T1ΔC | TTF-1-(1-223) |

RT-PCR: Total RNA was prepared from cultured cells using Trizol according to the manufacturer's protocol (InVitrogen, Carlsbad, Calif.). For RT-PCR, cDNA was generated by reverse-transcription (RT) using the total RNA. A 240 bp fragment of human Spdef was amplified using primers (forward 5′-TGT CCG CCT TCT ACC TCT CCT AC-3′ (SEQ ID NO: 18); reverse 5′-CGA TGT CCT TGA GCA CTT CGC-3′ (SEQ ID NO: 19)). A 407 bp fragment of mouse Spdef was amplified using primers (forward 5′-GTT GCC TGC TAC TGT TCC CAG ATG-3′ (SEQ ID NO: 20); reverse 5′-AAA GCC ACT TCT GCA COT TAC CAG-3′ (SEQ ID NO: 21)) under the following conditions: 94° C. for 5 minutes for 1 cycle, 30-35 cycles of 94° C. for 1 minute, annealing at 60° C. human Spdef and 58° C. for mouse Spdef for 30 seconds, 72° C. for 30-40 seconds, with a final extension cycle of 72° C. for 7 minutes. A 238 bp fragment of GAPDH was amplified using primers (forward, 5′-CTT CAC CAC CAT GGA GAA GGC-3′ (SEQ ID NO: 22); reverse, 5′-GGC ATG GAC TGT GGT CAT GAG-3′ (SEQ ID NO: 23)). The PCR products were resolved by gel electrophoresis on 1.5% agarose gels containing ethidium bromide. RT-PCR for Spdef, IL-13, IL-4, IL-6, TGF-α, Heparin Binding (HB)-EGF was performed using 2 μg total RNA from CCSP-rtTA/TRE2-Spdef mice treated with or without doxycycline (n=6) in each group using the primers described in Table 2. PCR products were identified on the gel-electrophoresis and scanned for quantification using ImageQuant (GE Healthcare Bio-Sciences Corp., Piscataway, N.J.). Lack of DNA contamination was verified by RT-PCR with presence or absence of reverse transcriptase.

TABLE 2

| Target | |
|---|---|
| Gene | Primer Sequences |
| Spdef | forward 5′-TTC CAG GAG CTG GGC GGT AA-3′ (SEQ ID NO: 30) |
| | reverse 5′-GGT CCA TGG TGA TAC AAG GGA CAT-3′ |
| | (SEQ ID NO: 31) |
| IL-13 | forward 5′-TGA GCA ACA TCA CAC AAG ACC AG-3′ (SEQ ID NO: 32) |
| | reverse 5′-GAG AAA GGA AAA TGA TCC ACA GC-3′ (SEQ ID NO: 33) |
| IL-4 | forward 5′-AAC CCC CAG CTA GTT GTC AT-3′ (SEQ ID NO: 34) |
| | reverse 5′-GCT CTT TAG GCT TTC CAG GA-3′ (SEQ ID NO: 35) |
| IL-6 | forward 5′-CCT CTG GTC TTC TGG AGT ACC AT-3′ (SEQ ID NO: 36) |
| | reverse 5′-GGC ATA ACG CAC TAG GTT TGC CG-3′ (SEQ ID NO: 37) |
| TFG-alpha | forward 5′-CCT GTT CGC TCT GGG TAT TGT GTT-3′ (SEQ ID NO: 38) |
| | reverse 5′-CGT GGT CCG CTG ATT TCT TCT CTA-3′ (SEQ ID NO: 39) |
| HB-EGF | forward 5′-GAC CAT GAA GCT GCT GCC GT-3' (SEQ ID NO: 40) |
| | reverse 5′-CGC CCA ACT TCA CTT TCT CTT C-3' (SEQ ID NO: 41) |

### Example 22

### Spdef Immunohistochemistry

Lung tissue was dissected from fetal and postnatal mice, fixed with 4% paraformaldehyde in PBS, dehydrated, and embedded in paraffin according to standard methods (Wert, et al. 2002. Dev. Biol. 242:75-87, which is incorporated by reference herein in its entirety). Immunostaining for Spdef was performed essentially as described previously (Zhou, et al. 1996. J. Histochem. Cytochem. 44:1183-1193, which is incorporated by reference herein in its entirety). AntiSpdef polyclonal guinea pig antibody was produced and used at 1:10,000. Foxj1, Sox17, Foxa2, CCSP (Scgb1a1), Muc5A/C and TTF-1 antibodies and immunohistochemistry have been described previously (Mucenski, et al. 2005. Am. J. Physiol. 289:L971-L979; Wan, et al. 2004. Development. 131:953-964; Park, et al. 2006. Dev. Biol. 294:192-202, each of which is incorporated by reference herein in its entirety).

### Example 23

### In Situ Hybridization for SPDEF

Riboprobes were synthesized from a 440 bp cDNA template for Spdef containing 50 nucleotides of the 5′ untranslated region and 389 nucleotides of coding sequences, subcloned into a pGEM 3Z transcription vector (Promega, Inc., Madison, Wis.). Riboprobes were synthesized using T7 (antisense) and SP6 (sense) polymerases and reagents contained in a commercial transcription kit (Riboprobe® In vitro Transcription Systems, Promega, Inc., Madison, Wis.) and labeled with 1,000 Ci/mmol of [35-S]-UTP and 800 Ci/mmol of [35-S]-CTP (Amersham Biosciences, Piscataway, N.J.). Single-stranded transcripts were separated from unincorporated nucleotides by column chromatography, precipitated in ammonium acetate and ethanol, and reconstituted in 200 mM DTT. For hybridization, the riboprobes were diluted in hybridization buffer to a final concentration of 5×10⁴ cpm/ul. Pretreatment, hybridization of tissue sections overnight (58° C.), and post-hybridization high stringency washes were performed as described previously (Wert, et al. 1993. Dev. Biol. 156:426-443, which is incorporated by reference herein in its entirety). Sections were dehydrated, dipped in Ilford K5 nuclear research emulsion (Polysciences, Inc., Warrington, Pa.), exposed for 2 to 6 weeks, and developed with Kodak D19 developer (Eastman Kodak, C., Rochester, N.Y.). The sections were then examined and photographed under dark field illumination with a Nikon Microphot FXA wide-field microscope.

### Example 24

### Spdef is Expressed in Pulmonary Epithelial Cells and is Co-Expressed with Sox17, Foxj1, and B-Tubulin

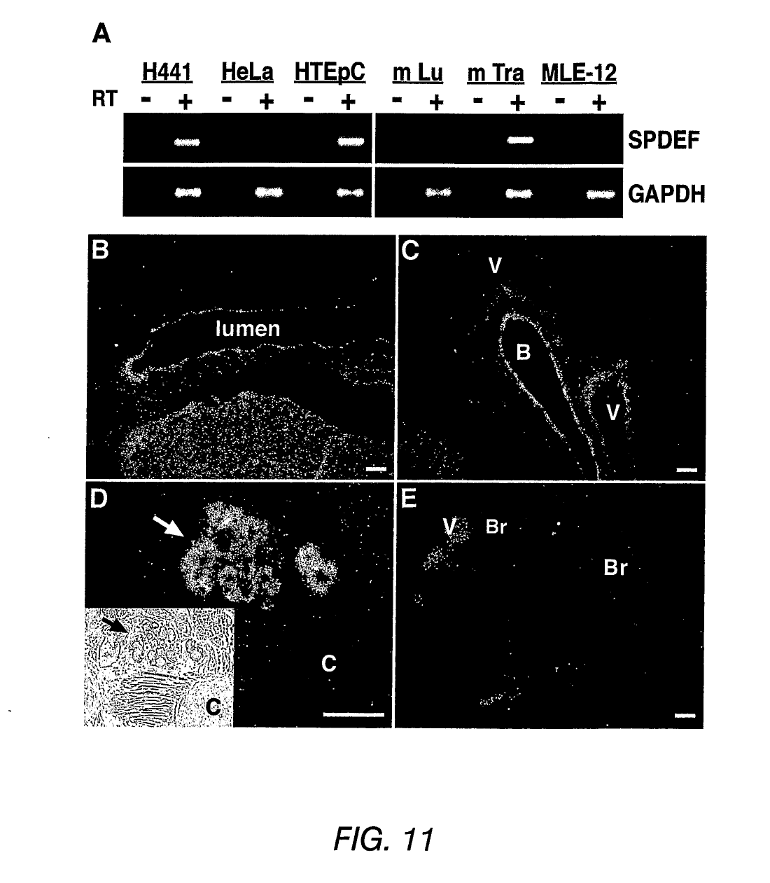

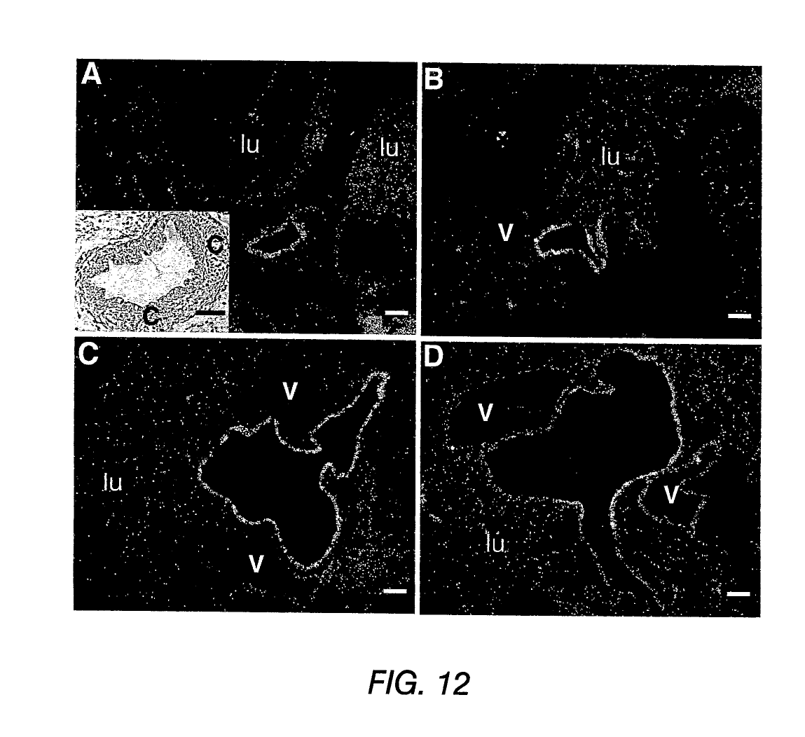

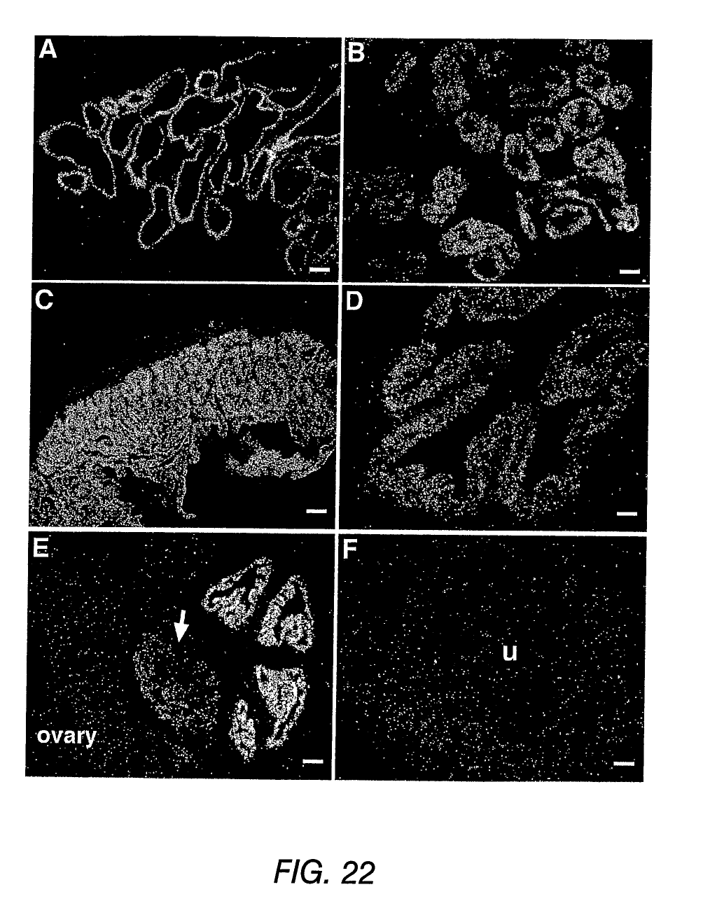

Mouse Spdef mRNA was detected in adult mouse lung and trachea, but not in MLE-12 cells, an SV40 immortalized mouse lung epithelial cell line with characteristics of type II alveolar cells, FIG. 11A. By in situ hybridization, Spdef mRNA was present in subsets of respiratory epithelial cells in extrapulmonary airways of the mouse lung from E17.5 to adulthood, FIGS. 11 and 12. In adult lung, Spdef mRNA was readily detected in subsets of epithelial cells in the trachea, extrapulmonary bronchi, and in epithelial cells of tracheal glands, FIG. 11B-D. Spdef mRNA was also present in H441, a human pulmonary adenocarcinoma cell line, and HTEpC, human tracheal epithelial cells (Cell Applications Inc., San Diego, Calif.), but not in HeLa cells, FIG. 11A. In situ hybridization demonstrated Spdef mRNA in epithelial cells of stomach, small intestine, caecum, colon, oviduct, dorsal and ventral prostate, coagulating gland and seminal vesicles, consistent with the reported distribution of Spdef mRNA (25) in the adult mouse, FIG. 22. An Spdef sense probe did not hybridize, FIG. 22.

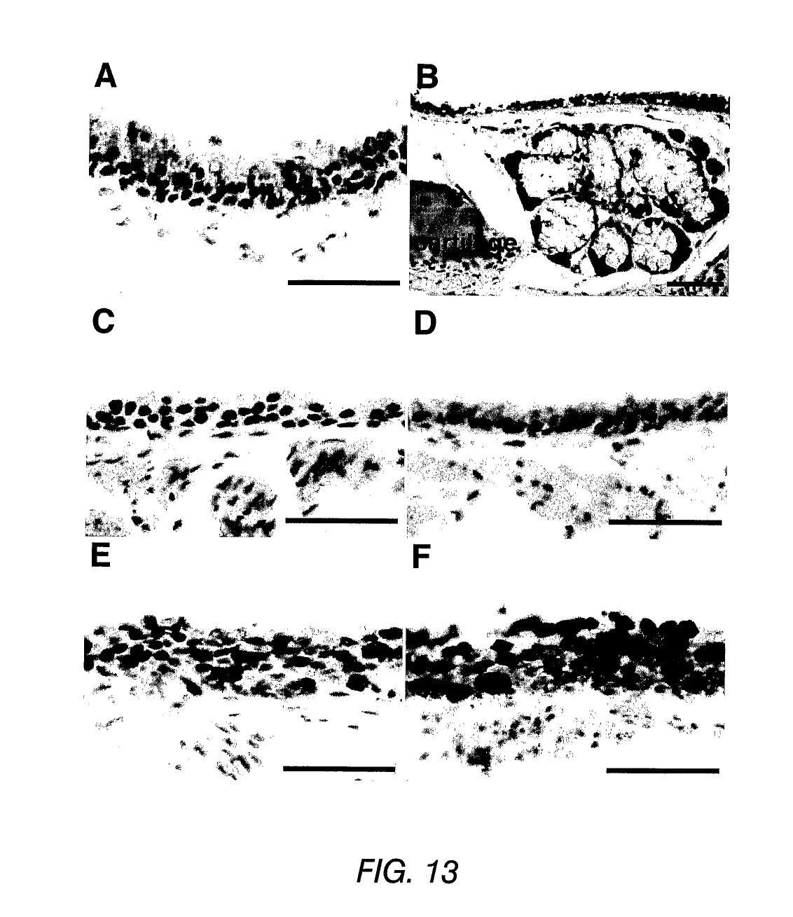

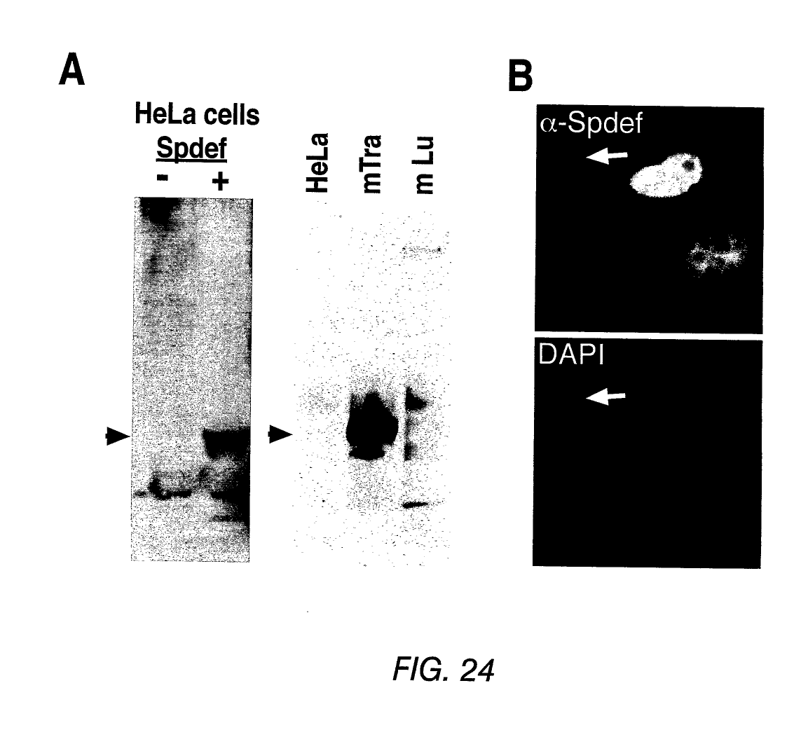

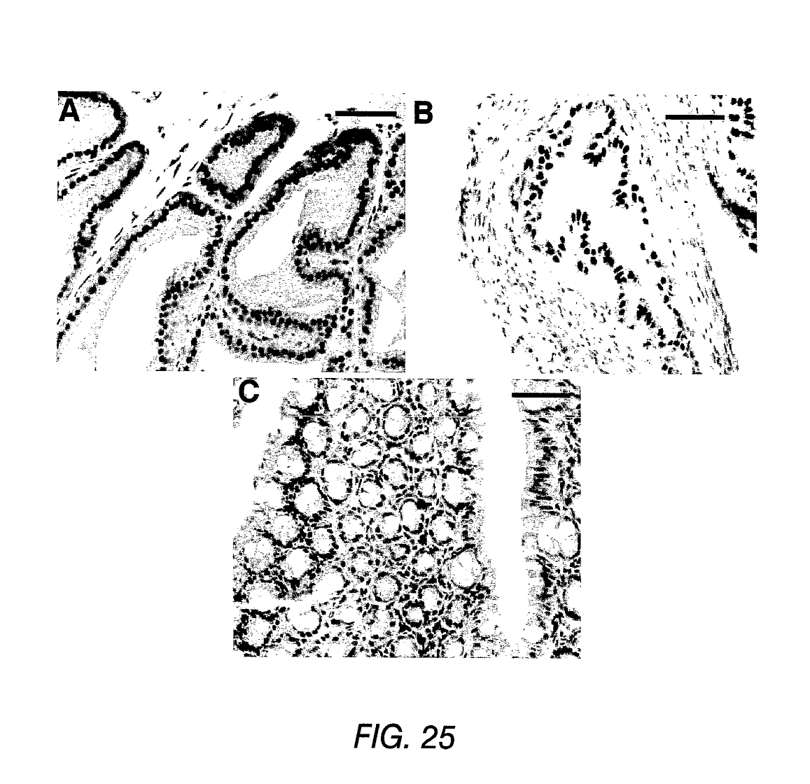

Polyclonal antisera were produced against recombinant Spdef that detected a single protein of approximately 37 kDa by immunoblot. Spdef antiserum immunostained HeLa cells transfected with a full length mouse Spdef cDNA, FIG. 24. Consistent with mRNA data, Spdef was detected in epithelial cells lining the adult mouse trachea (FIG. 13), but not in lung parenchyma. Nuclear staining for Spdef was observed in epithelial cells in trachea, bronchi, and tracheal glands consistent with the distribution of Spdef mRNA detected by in situ hybridization (FIG. 13A, B), and overlapping with sites of respiratory epithelial gene expression including TTF-1, Sox17, Foxj1, and Scgb1a1, FIG. 13C-F. Spdef mRNA and protein were observed in prostate, oviduct, colon, and seminal vesicles, FIGS. 22 and 25. In the adult lung, levels of staining intensity for Spdef varied in nuclei of respiratory epithelial cells lining conducting airways, FIG. 13A, B. High levels of Spdef mRNA and immunostaining were observed in epithelial cells of tracheal glands, FIGS. 11 and 13. During development, Spdef mRNA was first detected in conducting airway epithelial cells of the fetal mouse lung at E17.5, FIG. 12A. Thereafter, Spdef was present in epithelial cells lining extrapulmonary conducting airways, and was not detected by either in situ hybridization or immunohistochemistry in peripheral bronchiolar and alveolar epithelial cells, FIGS. 11-13. Timing and sites of expression of Spdef support its potential role in cell differentiation or gene expression in tracheal glands and proximal conducting airways, but not in peripheral airway or alveolar epithelial cells in the mouse.

In the lung, Spdef was restricted to ciliated respiratory epithelial cells where staining was colocalized with β-tubulin, Sox17, and Foxj1 transcription factors that are expressed primarily in ciliated cells in the conducting airways. Timing and sites of expression of Spdef supported its potential role in differentiation or regulation of gene expression in conducting airways, but not in alveolar type II cells, where Spdef expression was not detected by either immunochemistry or in situ hybridization. Thus, Spdef is selectively expressed in ciliated cells in conducting airways of the adult mouse lung.

### Example 25

### Spdef Interacts with TTF-1 and Regulates Gene Expression in Respiratory Epithelial Cells

The ability of Spdef to regulate potential transcriptional targets expressed at these cellular sites was assessed by transfection assays in vitro. To perform the transfection assays, the following method was used. HeLa cells were maintained as previously described (Liu, et al. 2002. J. Biol. Chem. 277:4519-4525, which is incorporated by reference herein in its entirety). In general, cells were seeded at 1×10⁵ per well in 6 or 12 well-plates and transfected with the plasmids using Lipofectamine-2000 (InVitrogen, Carlsbad, Calif.) or Effectene (Qiagen, Valencia, Calif.) according to the manufacturers' instructions. The amount of transfected DNA was kept constant by addition of corresponding amounts of the backbone plasmid. pCMV-β-galactosidase or pRL-TK encoding Renillar-luciferase was also transfected. After 36-48 hours of incubation, lysates were assayed for β-galactosidase and luciferase activities (Promega, Madison, Wis.). Light units were assayed by luminometry (Monolight 3010, Analytical Luminescence Laboratory, San Diego, Calif.). Firefly luciferase activities in relative light unit were normalized to β-galactosidase or Renillar-luciferase activity. All assays were performed in triplicate in at least three separate experiments. Sftpa (surfactant protein-A) is a host defense protein that is selectively expressed in epithelial cells of tracheal glands, bronchiolar and alveolar type II cells (Khoor, et al. 1993. J. Histochem. Cytochem. 41:1311-131926).

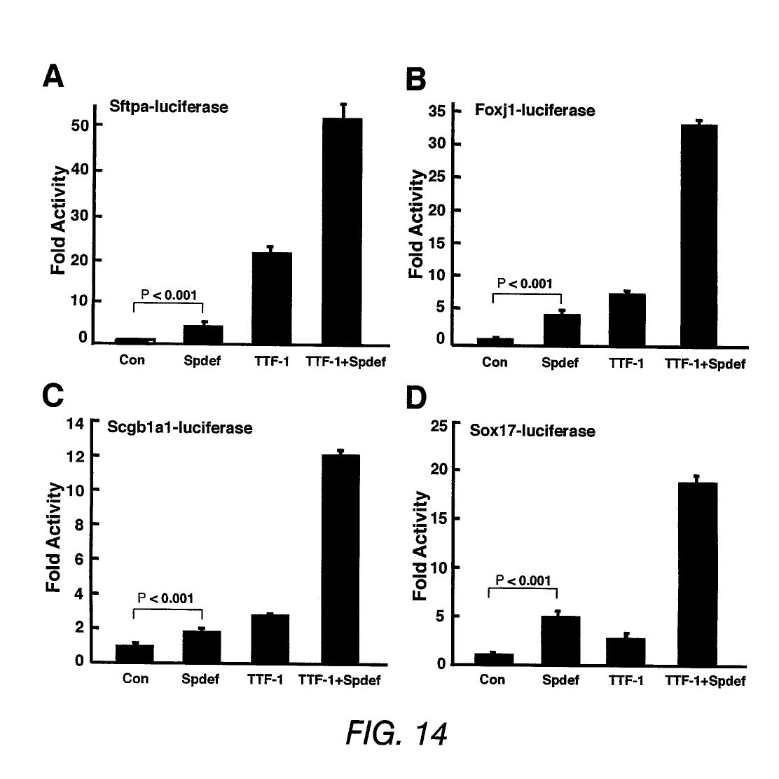

Spdef enhanced the activity of the Sftpa promoter in vitro, FIG. 14A. Cotransfection of Spdef with TTF-1 further activated the Sftpa promoter, FIG. 14A. Potential Spdef binding motifs GGAAIT (Karim, et al. 1990. Genes Dev. 4:1451-1453, which is incorporated by reference herein in its entirety) were identified in the Sftpa promoter; however, repeated attempts to bind recombinant Spdef or the DNA binding domain of Spdef to consensus Spdef elements in the Sftpa promoter (a.a. 247-335) by EMSA were unsuccessful. Deletion of these potential sites in the Sftpa promoter did not inhibit Spdef effects in the transfection assays.

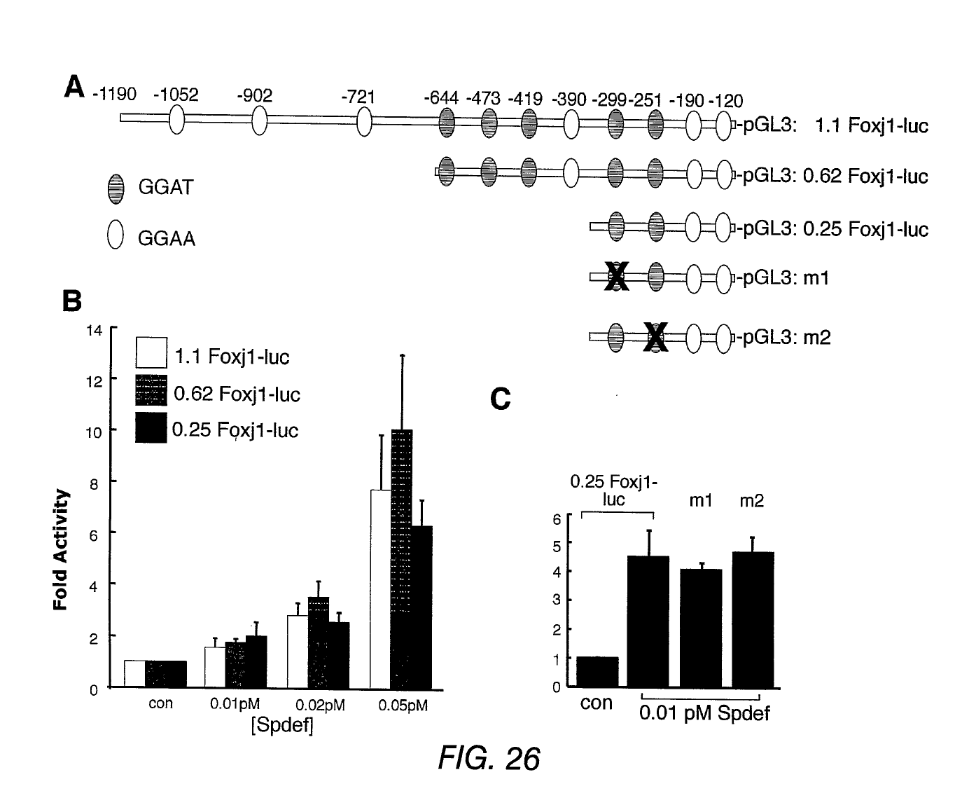

Since Spdef was expressed in conducting airway epithelial cells, tests were conducted to determine whether Spdef regulated the promoters of other genes expressed selectively in the proximal airways, including Foxj1, Sox17, Scgb1a1, and MUC5A/C. Spdef acted synergistically with TTF-1 on the promoters of Foxj1, Scgb1a1 and Sox17 (FIG. 14B-D), but did not activate the MUC5A/C gene promoter (data not shown). Direct binding of the recombinant Spdef homeodomain or full length Spdef recombinant protein to the Foxj1 elements or to a previously reported consensus Spdef binding site identified in the PSA gene by EMSA (Oettgen, et al. 2000. J. Biol. Chem. 275:1216-1225, which is incorporated by reference herein in its entirety) was not demonstrated. Deletion and mutation of several potential Spdef binding sites identified in the Foxj1 promoter did not block its activation by Spdef, FIG. 26.

### Example 26

### Spdef Activates Sox17 and Foxj1 in Postnatal Lung

In the postnatal lung, Spdef was co-expressed with Sox17, Foxj1, Foxa1, Foxa2, and β-tubulin in ciliated respiratory epithelial cells. The effects were assessed of Spdef on the promoters expressed selectively in ciliated cells. Spdef activated both the Sox17 and Foxj1 promoters in vitro. Effects of Spdef were further activated by co-transfection with Ttf-1 and Gata-6 in HeLa and H441 cells. Electromobility shift assays demonstrated that Spdef bound to a cis-acting element within the Sox17 and Foxj1 genes. Addition of Spdef antibody caused a shift in migration in the EMSA, consistent with its direct interaction with the promoter.

### Example 27

### Use of Mammalian Two Hybrid System Assays to Determine that Spdef Interacts with the C-Terminal Domain of Ttf-1

Because of the observed synergistic response of several promoters to TTF-1 and Spdef, potential interactions between TTF-1 and Spdef were assessed by mammalian two-hybrid assays in HeLa cells and by pull-down assays in vitro.

To perform the mammalian two hybrid system assays, the following method was used. Spdef, TTF-1, and TTF-1 mutant eDNAs were generated by PCR and cloned into the vectors pACT and pBIND in the mammalian two hybrid system kit (Promega, Madison, Wis.) between BamHI/XbaI sites (Table 1).

HeLa cells were plated in 6 well plates at a density of 5×10⁴, 24 hours before cells were transfected with Effectene transfection reagent (Qiagen, Valencia, Calif.). Cells were transfected with of pG5-luc and pACT-Spdef, and pBIND-TTF-1, and pBIND-TTF-1 mutants at 0.04 pMol of each vector per well. Forty-eight hours after transfection, cell lysates were assayed for firefly luciferase (from pG5-luc) and Renillar luciferase (pBIND) activities using the Dual-Luciferase Reporter Assay System (Promega, Madison, Wis.). Values were normalized compared to empty vector control. All transfection experiments were performed in triplicate, and repeated at least three times with similar results.

Interactions of TTF-1 with Spdef were mediated by the C-terminal domain of TTF-1 and did not require the TTF-1 homeodomain as assessed by both mammalian two-hybrid assays and co-immunoprecipitation assays with GST-Spdef. Thus, activation of target genes by Spdef can be mediated, at least in part, by its interactions with TTF-1, known to bind and activate the promoters of a number of genes selectively expressed in the respiratory epithelium, including Sftpb, Sftpa, Sftpc and Scgb1a1 (Bohinski, et al. 1994. Mol. Cell. Biol. 14:5671-5681; Bruno, et al. 1995. J. Biol. Chem. 270:6531-6536. Erratum in: J. Biol. Chem. 1995. 270, 16482; Kelly, et al. 1996. J. Biol. Chem. 271:6881-6888; Ray, et al. 1996. Mol. Cell. Biol. 16:2056-2064, each of which is incorporated by reference herein in its entirety).

### Example 28

### Conditional Expression of Spdef in the Mouse Lung

Mice expressing Spdef were generated by cloning the full length mouse Spdef coding sequence (including Kozak sequences) at the SalI and XbaI sites in the pTRE2 vector (Clontech, Mountainview, Calif.). Primers were synthesized for the Spdef coding sequence with SalI and XbaI ends (underlined) (forward, 5′-CCC GGG GTC GAC CGC AGC ATG GGC AGT GCC AGC CCA GG-3′ (SEQ ID NO.: 24); reverse, 5′-CCC GGG TCT AGA TCA GAC TGG ATG CAC AAA TTG GTAG-3′ (SEQ ID NO.: 25) respectively). Amplification of PCR products was performed as follows: denaturation at 94° C. for 2 minutes; 35 cycles of denaturation at 94° C. for 30 seconds, annealing at 60° C. for 30 seconds, and extension at 72° C. for 1 minute, followed by a 7 minute extension at 72° C. Following amplification, PCR products were digested with SalI and XbaI and cloned into pTRE2 digested with SalI and XbaI. pTRE2-Spdef clones were confirmed by DNA sequencing of both strands. pTRE2-Spdef was digested with AatII and SapI and the resulting fragment containing Spdef was microinjected into FVBN embryos by the Children's Hospital Research Foundation Transgenic Animal Facility. Founder mice were identified by PCR. Briefly, transgenic mice were identified using PCR primers specific for TRE2-Spdef transgene (forward Spdef coding sequence 5′-TGA ACA TCA CAG CAG ACCC-3′ (SEQ ID NO.: 26) ; reverse, pTRE2 vector sequence 5′-TCT TCC CAT TCT AAA CAA CACC-3′ (SEQ ID NO.: 27)). Amplification of PCR products was performed as follows: denaturation at 94° C. for 2 minutes; 35 cycles of denaturation at 94° C. for 30 seconds, annealing at 60° C. for 30 seconds, and extension at 72° C. for 30 seconds, followed by a 7 minute extension at 72° C. Four founder mice were identified as containing Spdef and pTRE2 sequences. For lung specific, doxycycline-induced recombination, the Clara cell secretory protein/reverse tetracycline transactivator (CCSP-rtTA) transgene was used (30, 31). CCSP-rtTA mice were bred with each of the four founding mice containing Spdef. Offspring were identified using PCR primers specific for the CCSP-rtTA and TRE2-Spdef transgenes.

### Example 29

### Co-Precipitation and Immunoblot Analysis

To make the GST fusion protein, mouse Spdef cDNA was amplified using primers: forward, 5° -GGG CAC GGA TCC ATG GGC AGT GCC AGC CCA GG-3′ (SEQ ID NO.: 28); reverse, 5′-CCC GGG GTC GAC TCA GAC TGG ATG CAC AAA TTG GTAG-3′ (SEQ ID NO.: 29). The PCR product was digested with BamHI and SalI (underlined) and subcloned into the pGEX4T-1 GST vector (Amersham Biosciences, Piscataway, N.J.) and transformed into B21 bacteria for protein expression. After 5 hours of incubation at 37° C. in 1 mM isopropyl-β-D-thiogalactoside, bacteria were harvested and stored at −80° C. overnight. Cells were resuspended in 1× PBS followed by sonication and treatment with 1% Triton X-100 for 30 minutes on ice and centrifuged at 12,000×g for 10 minutes. The protein was purified on glutathione-Sepharose 4B. Eluted Spdef was then dialyzed against 10 mM Tris-HCl pH 8.0 at 4° C. and stored at −80° C. 3×FLAG-TTF-1 and 3×FLAGΔ3 (amino acids 159-372) were described previously (Park, et al. 2004. J. Biol. Chem. 279:17384-17390, which is incorporated by reference herein in its entirety). 3×FLAGΔ14 (amino acids 1-221) was generated by PCR amplification of the coding region of TTF-1 deletion Δ14 and subcloned into the HindIII/BamHI sites of the 3×FLAG CMV-10 vector (Sigma, St. Louis, Mo.). HeLa cells were plated at a density of 1×10⁵ in 6 well plates and transfected with 3×FLAG-TTF-1, 3×FLAGΔ14, and 3×FLAGΔ3. Forty-eight hours after transfection with Effectene, nuclear extracts were prepared as previously described (Park, et al. Id.). Coprecipitation was performed by incubating GST or GST-Spdef proteins bound to glutathione-agarose beads with nuclear extract prepared from HeLa cells transfected with FLAG-TTF-1 constructs (Park, et al. Id.). Proteins were eluted by resuspending the beads directly in SDS-PAGE sample buffer and heating at 100° C. for 5 minutes before loading on gels. Proteins from lysates of HeLa cells transfected with an Spdef expression plasmid, mouse trachea and lung were prepared as above and separated by SDS-PAGE. Proteins were transferred to nitrocellulose and detected with guinea pig polyclonal antibody generated against recombinant mouse Spdef.

### Example 30

### Dust Mite Allergen Exposure, IL-13 and Stat-6−/− Transgenic Models

Animals were maintained and handled under Institutional Animal Care and Use Committee-approved procedures and the Guide for the Care and Use of Laboratory Animals (Institute of Laboratory Animal Resources, National Research Council). Control C57B16 and Stat-6^{-/-} mice were treated intratracheally with IL-13 as previously described (Wan, et al. 2004. Development. 131:953-964, which is incorporated by reference herein in its entirety), lung tissue kindly provided by Dr. R. Finkelman, University of Cincinnati. IL-13 was expressed under conditional control in CCSP-rtTA, otet-CMV-IL-13 transgenic mice as previously reported (Wan, et al. Id.), lung tissue kindly provided by Dr. M. Rothenberg and Patricia Fulkerson. The expression of the IL-13 transgene was induced by treatment of the mice with doxycycline. On day 0 and day 7, 4-week-old IL-13-deficient mice on BALB/c background (kindly provided by Dr. Andrew McKenzie, Medical Research Council Laboratory of Molecular Biology, Cambridge, United Kingdom) (McKenzie, et al. 1999. Immunity 9:423-432, which is incorporated by reference herein in its entirety) and 3 to 5-week-old wild type BALB/c mice (Jackson Laboratory, Bar Harbor, Me.) were sensitized intraperitoneally with 10 μg of house dust mite (HDM) (Greer Laboratories, Lenoir, N.C.) in 100 μl phosphate-buffered saline (PBS) or equivalent amount of PBS alone. On day 14 and day 21, mice were anesthetized with the mixture of ketamine and xylazine (Phoenix Pharmaceutics Inc., St Joseph, Mo.) intraperitoneally, challenged intratracheally with 100 μg HDM in 50 μl PBS or PBS alone. On Day 26, the lung tissue was harvested, fixed with 10% neutral formalin (Sigma-Aldrich Corp, St. Louis, Mo.). The lung tissue was embedded in paraffin and five μm sections were cut for histological analysis.

### Example 31

### Spdef Caused Goblet Cell Hyperplasia In Vivo

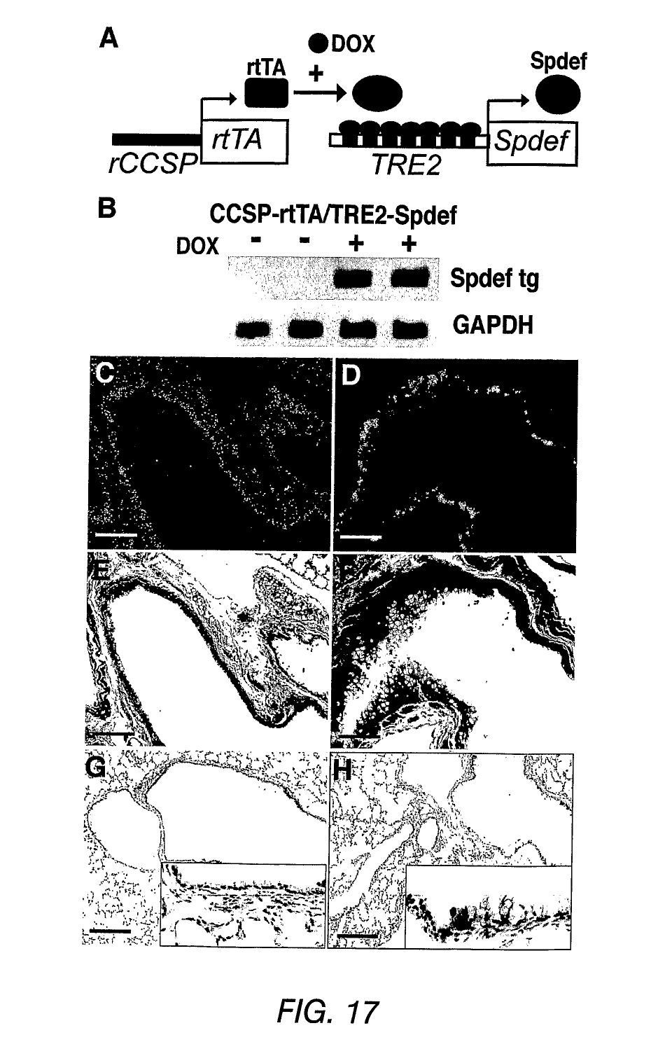

Since Spdef was expressed in a subset of epithelial cells in the trachea, bronchi, and tracheal glands, and stimulated transcriptional activity of genes normally expressed in proximal airway epithelial cells in vitro, its role was assessed in vivo. Spdef was conditionally expressed under control of CCSP-rtTA, FIG. 17A. Spdef transgene mRNA was not detected in lung unless the mice were treated with doxycycline, FIG. 17B. In situ hybridization and immunohistochemistry demonstrated the induction of Spdef mRNA and protein in subsets of cells in the respiratory epithelium lining conducting airways (FIG. 17D, H) and alveoli (latter not shown), consistent with the activity of the CCSP (Scgb1a1) promoter in this mouse line, which selectively directs expression to Clara cells in the conducting airways (Perl, et al. 2002. Transgenic Res. 11:21-29; Perl et al. 2005. Am. J. Respir. Cell. Mol. Biol. 33:455-462, each of which is incorporated by reference herein in its entirety).

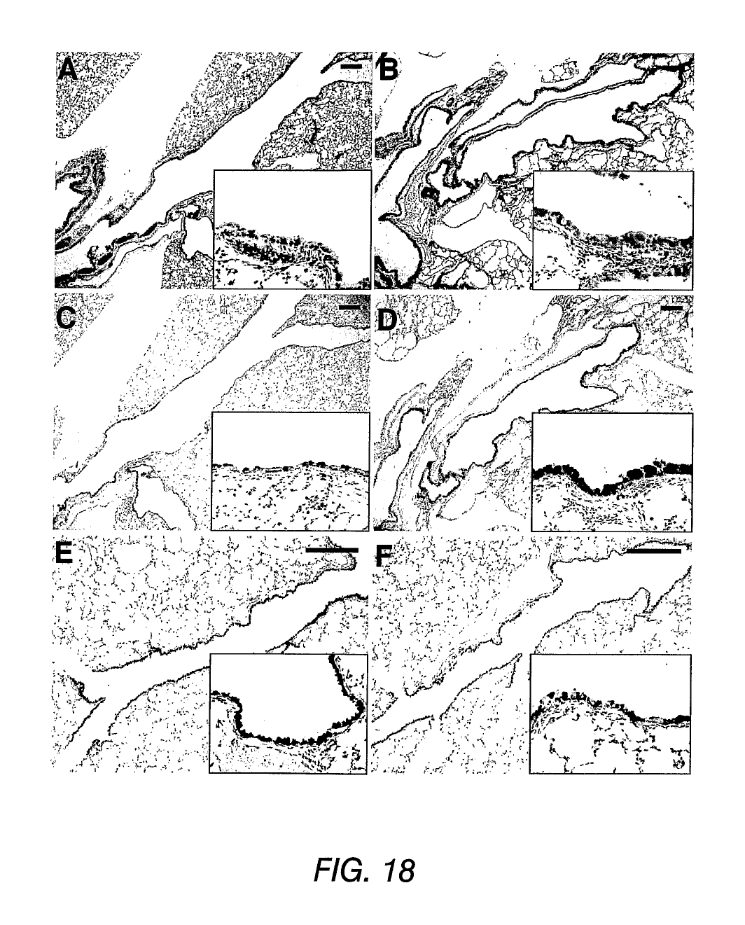

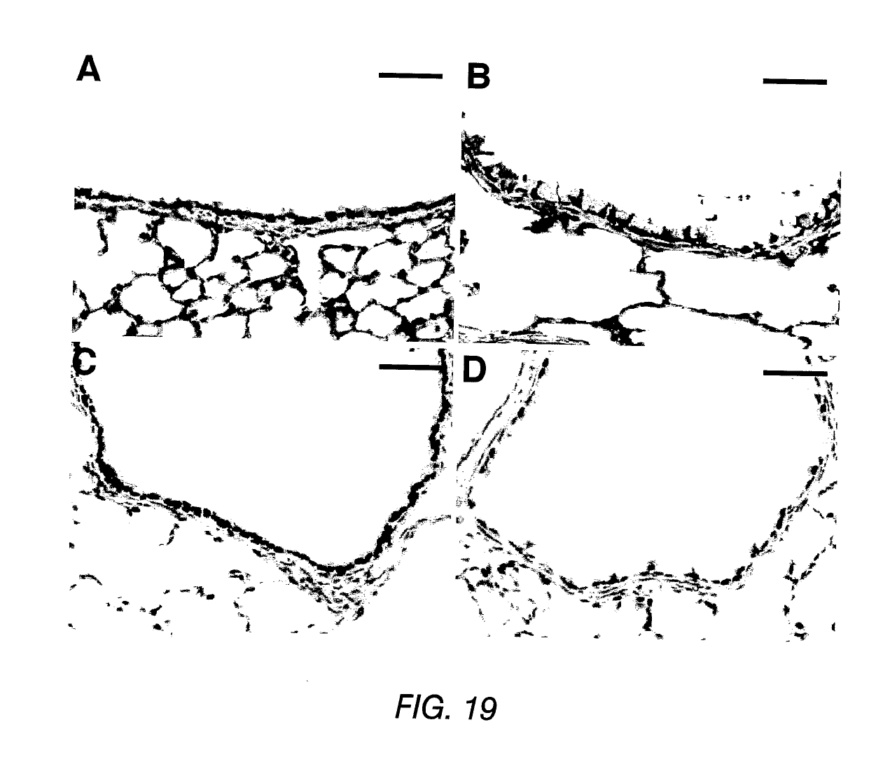

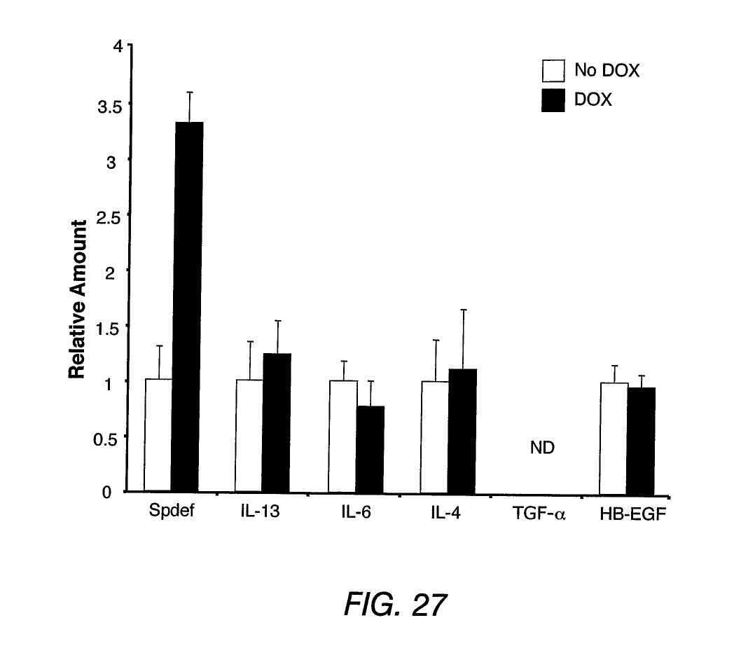

Spdef caused goblet cell differentiation in extrapulmonary and intrapulmonary airways, FIGS. 17, 18, 19. Findings were consistent in two independent TRE2-Spdef mouse lines and were dependent upon doxycycline. Alcian-blue staining (FIG. 18A, B) and immunostaining for MUC5A/C (FIG. 18C, D) were increased at the sites of Spdef expression in the trachea and bronchi and in peripheral airways, including smaller bronchioles that normally lack goblet cells. Goblet cell hyperplasia occurred in the absence of inflammation, leukocytic infiltration or altered expression of TGF-α, HB-EGF, IL-4, IL-6, and IL-13 mRNAs, Table 2 and FIG. 27. CCSP staining was decreased in regions lined by goblet cells (FIG. 18E, F), whereas the staining pattern for Foxj1, a ciliated cell marker, was not altered, FIG. 19A, B. Since the CCSP-rtTA driven Spdef transgene is expressed selectively in Clara cells, paucity of CCSP (Scgb1a1) staining, and the presence of goblet cell hyperplasia seen in vivo are consistent with a cell autonomous effect of Spdef on the differentiation of Clara cells into goblet cells. Since loss of Foxa2 was previously shown to cause goblet cell differentiation (Wan, et al. 2004. Development. 131:953-964, which is incorporated by reference herein in its entirety), the effect of Spdef on Foxa2 expression was assessed. Foxa2 staining was absent at sites of goblet cell hyperplasia, FIG. 19C, D. Phospho-histone 3 (pH3) staining was used to identify proliferating cells. The ectopic goblet cells did not stain for pH3, supporting the concept that expression of Spdef in the airway epithelium influenced cell differentiation rather than proliferation (data not shown).

### Example 32

### Induction of Spdef Expression in Mouse Models with Goblet Cell Hyperplasia

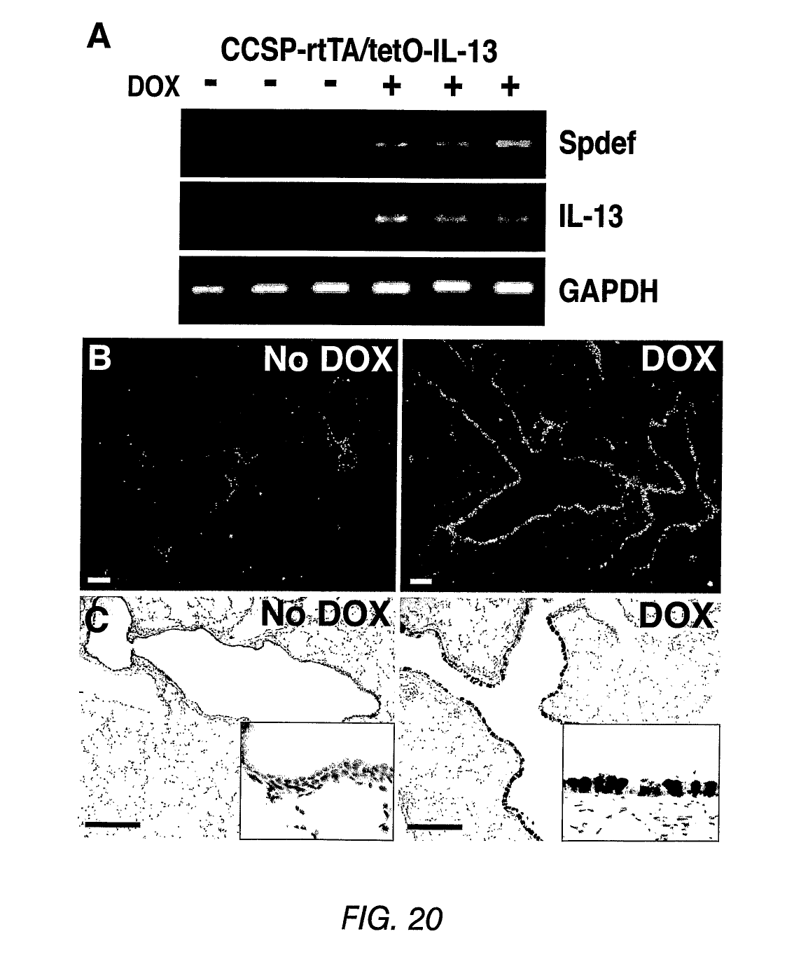

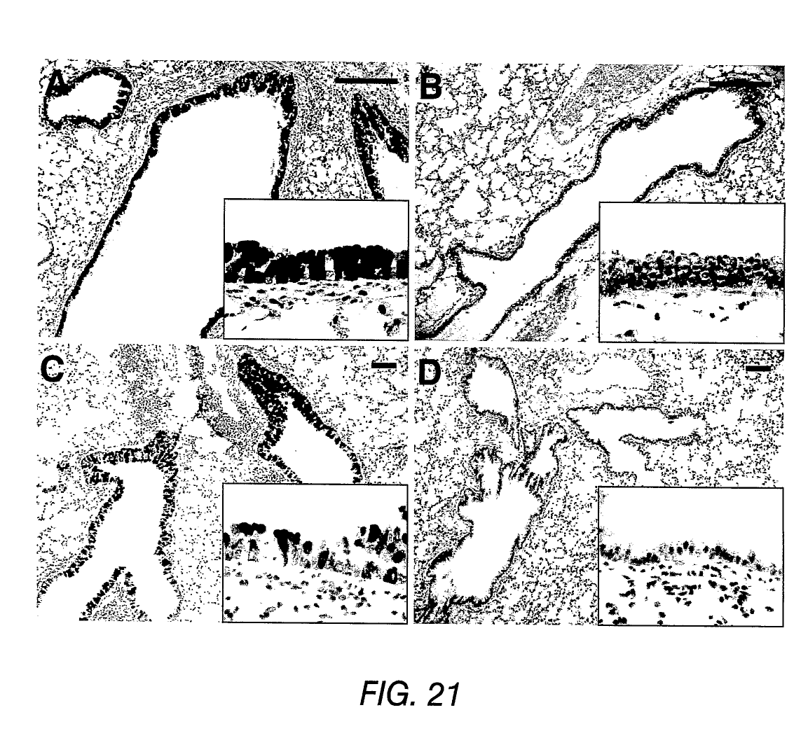

Increased expression of either IL-4 or IL-13 (32-35) and allergen challenge (36) cause goblet cell hyperplasia in vivo. It was tested whether increased Spdef was associated with goblet cell hyperplasia in mice expressing IL-13 in Clara cells under conditional control of doxycycline. Increased Spdef staining and mRNA were associated with goblet cell hyperplasia in conducting airways in adult mice as assessed by RT-PCR, in situ hybridization and immunostaining, FIG. 20. Spdef staining was observed in both the cytoplasm and nuclei of epithelial cells in conducting airways. Likewise, intratracheal IL-13 caused goblet cell hyperplasia in association with increased Spdef staining in control but not in Stat-6^{-/-} mice, FIG. 21A, B. Goblet cell hyperplasia and increased Spdef staining were observed following repeated intratracheal administration of dust mite allergen to wild type mice but was not observed in treated IL-13^{-/-} mice, FIG. 21C, D. IL-13 and allergen exposure increased Spdef mRNA and extended its expression in both extra- and intrapulmonary airways.

### Example 33

### Administration of Nucleic Acid Encoding Spdef to activate Sox17 in a Damaged Lung

An individual with pulmonary damage is treated with an aerosol administration of an adenoviral vector encoding human Spdef, operably linked to a suitable promoter. Once the composition enters the cell, Spdef protein is produced. The Spdef protein in turn activates the transcription of Sox17, and a cascade of several proteins involved in pulmonary repair is produced. By use of this method, the damaged pulmonary tissue heals rapidly.

### Example 34

### Determination of an Effective Amount of Sox17 Nucleic Acid for Patient Treatment

100 patients with pulmonary damage due to inhalation of tobacco smoke are identified. A nucleic acid vector containing the Sox17 sequence is prepared and formulated into a liposomal composition according to Legace et al. (J Microencapsulation, 8:53-61 (1991), which is incorporated by reference herein in its entirety). To determine the optimal amount of Sox17 nucleic acid composition to administer, the patients are given aerosol delivery devices that are each set to deliver different amounts of formulation. Patients will receive either 0 μg, 0.1 μg, 1.0 μg, 10 μg, 100 μg, or 1 mg of nucleic acid vector per day. The patients self administer the composition once per day. After 2 weeks, tissue samples are taken and analyzed for effectiveness of the treatment. Additional tests are performed to determine the optimal number of administrations per day. By use of this method, an optimal range of Sox17 nucleic acid to be administered is determined.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably can be practiced in the absence of any element or elements, limitation or limitations which is not specifically disclosed herein. Thus, for example, in each instance herein any of the terms “comprising”, “consisting essentially of and “consisting of can be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention disclosed. Thus, it should be understood that although the present invention has been specifically disclosed by preferred embodiments and optional features, modification and variation of the concepts herein disclosed can be resorted to by those skilled in the art, and that such modifications and variations are considered to be within the scope of this invention as defined by the disclosure.

1. A pharmaceutical composition effective in treating lung injury in a mammal, comprising an agent capable of upregulating expression of Sox17 protein or an active fragment of said Sox17 protein, in admixture with a pharmaceutically acceptable excipient.

2. The pharmaceutical composition of claim 1, wherein said agent comprises a nucleic acid molecule comprising at least 90%, 95%, 97%, 98%, or 99% homology to a nucleic acid encoding human Sox17 protein or an active fragment of said human Sox17 protein.

3. The pharmaceutical composition of claim 2, wherein said nucleic acid molecule is at least about 50, 100, 150, 200, 250, 500, 800, 1000, or 1240 nucleotides in length.

4. A method for the treatment of pulmonary injury, comprising administering a composition comprising an agent capable of upregulating expression of Sox17 protein or an active fragment of said Sox17 protein to an individual.

5. The method of claim 4, wherein the expression of β-catenin is activated.

6. The method of claim 4, wherein the expression of Stat-3 is activated.

7. The method of claim 4, wherein said composition is administered intratracheally.

8. The method of claim 4, wherein said composition is administered by aerosolization.

9. The method of claim 4, wherein said composition is administered using a nebulizer

10. The method of claim 4, wherein said pulmonary injury is a chemically-induced lung injury.

11. The method of claim 4, wherein said pulmonary injury is caused by a pulmonary disease.

12. The method of claim 4, wherein said pulmonary injury is caused by at least one condition selected from the group consisting of: pulmonary fibrosis, sarcoidosis, asbestosis, aspergilloma, aspergillosis, pneumonia, pulmonary tuberculosis, rheumatoid lung disease, bronchiectasis, bronchitis, bronchopulmonary dysplasia, interstitial lung disease, occupational lung disease, emphysema, cystic fibrosis, acute respiratory distress syndrome (ARDS), asthma, chronic bronchitis, and COPD (chronic obstructive pulmonary disease).

13. The method of claim 4, wherein said pulmonary injury is caused by a viral, bacterial, or fungal disease.

14. The method of claim 4, further comprising introducing Stat-3 protein or fragment thereof, or a nucleic acid encoding a Stat-3 protein or fragment thereof.

15. The method of claim 4, further comprising introducing β-catenin protein or fragment thereof, or a nucleic acid encoding a β-catenin protein or fragment thereof.

16. The method of claim 4, wherein said composition comprises a nucleic acid molecule having at least 90%, 95%, 97%, 98%, or 99% homology to SEQ ID NO: 5 or a fragment thereof, in admixture with a pharmaceutically acceptable excipient.

17. A method of inducing respiratory epithelial cell differentiation, comprising administering a nucleic acid molecule encoding a Sox17 polypeptide or active fragment therof.

18. A method of inducing pulmonary progenitor cells to enhance pulmonary repair, comprising administering a nucleic acid molecule encoding a Sox17 polypeptide or fragment thereof.

19. The method of claim 4, wherein said agent comprises a nucleic acid encoding mammalian Sox17 protein or an active fragment of said Sox17 protein, and wherein said composition is administered to a human in an amount effective to reduce the symptoms of said pulmonary injury.

20. The method of claim 4, wherein said agent is an Spdef protein, an active fragment of an Spdef protein, or a nucleic acid encoding Spdef.

21. A method of identifying a compound for the treatment of pulmonary injury, by
obtaining a mammalian cell;
testing said cell by adding at least one test compound; and
determining whether Sox17 expression is increased;
whereby an increase in Sox17 expression indicates that the test compound is potentially useful for the treatment of pulmonary injury.

22. The pharmaceutical composition of claim 1, wherein said agent comprises a nucleic acid molecule encoding Spdef protein or an active fragment of said Spdef protein.

23. The pharmaceutical composition of claim 1, wherein said agent comprises a nucleic acid molecule having at least 90%, 95%, 97%, 98%, or 99% homology to a nucleic acid molecule encoding human Spdef protein or an active fragment of said Spdef protein.

24. The pharmaceutical composition of claim 22, wherein said nucleic acid molecule is at least about 50, 100, 150, 200, 250, 500, 800, 900, or 1000 nucleotides in length.

25-31. (canceled)

32. The method of claim 4, wherein said agent comprises a nucleic acid molecule encoding mammalian Spdef protein or an active fragment of said Spdef protein, and wherein said composition is administered to a human in an amount effective to reduce the symptoms of said pulmonary injury.

33. A method of identifying a compound for the treatment of pulmonary injury, by
obtaining a mammalian cell;
testing said cell by adding at least one test compound; and
determining whether Spdef expression is increased;
whereby an increase in Spdef expression indicates that the test compound is potentially useful for the treatment of pulmonary injury.

34. The pharmaceutical composition of claim 1, wherein said agent comprises a nucleic acid molecule encoding Sox17 protein or an active fragment of said Sox17 protein.
